(19) United States
(12) Patent Application Publication
(10) Pub. No.: US 2020/0121342 A1
(43) Pub. Date: Apr. 23, 2020

(54) REMOVAL DEVICE

(71) Applicant: Solace Therapeutics, Inc., Framingham, MA (US)

(72) Inventors: Roy H. Sullivan, III, Uxbridge, MA (US); William H. Gruber, Southborough, MA (US); Albert Chum-Chi Chin, Newton, MA (US); Nicole M. Shugrue, Franklin, MA (US); Kevin G. Connors, Wellesley, MA (US); Matthew J. Witney, Upton, MA (US)

(21) Appl. No.: 16/557,555

(22) Filed: Aug. 30, 2019

Related U.S. Application Data
(60) Provisional application No. 62/725,210, filed on Aug. 30, 2018.

(51) Int. Cl. A61B 17/29; A61B 1/307; A61B 17/34; A61F 2/00

(52) U.S. Cl. CPC A61B 17/29; A61B 1/307; A61B 2017/2932; A61F 2/0045; A61F 2/0027; A61B 17/3468

(57) ABSTRACT

Method and system for treating a patient using a compressible, pressure-attenuating device. According to one embodiment, the system is used to treat urinary tract disorders and comprises an access device, a delivery device, a pressure-attenuating device, and a removal device. The access device can be used to create a passageway to an anatomical structure, such as the patient's bladder. The delivery device can be inserted through the passageway created by the access device and can be used to deliver the pressure-attenuating device to the anatomical structure. The removal device can be inserted through the passageway created by the access device and can be used to view the bladder and/or to capture, to deflate and to remove the pressure-attenuating device.

### CROSS-REFERENCE TO RELATED APPLICATIONS

This application claims priority to U.S. Provisional Application No. 62/725,210, filed on Aug. 30, 2018, the entirety of which is hereby incorporated by reference herein for all purposes.

### BACKGROUND

### Field

The present disclosure relates to methods and systems for performing medical procedures on anatomical structures of the body. Such medical procedures may involve, for example, attenuating transient pressure waves in anatomical structures of the body, for example, by implanting a compressible pressure-attenuating device in an anatomical structure of the body that is subjected to such pressure waves.

### Description of the Related Art

Pressure waves are known to propagate through incompressible fluids in various anatomical structures of the body. These pressure waves may be caused by normally-occurring events within the body, such as a beating heart, breathing in the lungs, peristalsis actions in the GI tract, and movement of the muscles of the body. Alternatively, these pressure waves may be caused by sudden events, such as coughing, laughing, external trauma to the body, and movement of the body relative to gravity. As the elasticity of the surrounding tissues and organs, sometimes referred to as compliance, decreases, the propagation of these pressure waves increases. These pressure waves have many undesirable effects ranging from discomfort to stress on the organs and tissue to fluid leakage to renal failure to stroke to heart attack to blindness.

Urinary tract disorders, such as frequency, urgency, incontinence, and cystitis, are a widespread problem in the United States and throughout the world, affecting people of all ages, both physiologically and psychologically. Urine is primarily composed of water and is a virtually incompressible fluid in the typical pressure ranges that are present within the human bladder. The relationship between the maximum urethral pressure and the intravesical pressure for normal voiding of the bladder is well-defined. During normal voiding, relaxation of the urethra occurs before the detrusor muscle contracts to cause the intravesical pressure to exceed the urethral pressure.

Intravesical pressure spikes often result from volumetric tissue displacement in response to gravity, muscular activity or rapid acceleration. The lack of compliance of the bladder and the urine contained in the bladder with respect to events of high frequency, high intensity and short wavelength results in minimal fluidic pressure attenuation of the higher frequency pressure wave(s) and results in high intravesical pressures that are directly transmitted to the bladder neck and urethra, which may or may not cause detrusor contractions. Under these conditions, the urethra may act as a volumetric pressure relief mechanism, allowing a proportional volume of fluid to escape the bladder, thereby lowering the intravesical pressure to a tolerable level. The urethra has a maximum urethral pressure value, and when the intravesical pressure exceeds the maximum urethral pressure, fluid will escape the bladder. Under these conditions, nerve receptors in the bladder and/or bladder neck and/or trigone trigger a detrusor contraction that may lead to micturition(frequency) or may subside without micturition(urgency) or may lead to the intravesical pressure exceeding the maximum urethral pressure resulting in fluid escaping the bladder (stress incontinence).

For the vast majority of patients suffering from problems of urinary tract disorders, such as frequency, urgency, stress and urge incontinence and cystitis, the cause and/or contributor to bladder dysfunction is a reduction of overall dynamic bladder compliance, as opposed to a reduction of steady-state bladder compliance. These patients may often have bladders that are compliant in steady-state conditions but that become non-dynamically compliant when subjected to external pressure events having a short duration of, for example, less than 5 seconds or, in some cases, less than 0.5 seconds. Reduction in dynamic compliance of the bladder is often caused by aging, use, distention, childbirth and trauma. In addition, the anatomical structure of the bladder in relation to the diaphragm, stomach, and uterus (for women) causes external pressure to be exerted on the bladder during physical activities, such as talking, walking, laughing, sitting, moving, turning, and rolling over. For a patient suffering from stress incontinence due to lack of dynamic compliance in the bladder, when the intravesical pressure exceeds the maximum urethral pressure, leakage occurs.

In light of the foregoing, a number of attempts have been made to combat urinary tract disorders. One such attempt involves the use of an indwelling catheter connected to a collection bag with a clamping device on the catheter. Indwelling catheters, however, have a number of drawbacks. For instance, there is an infection risk associated with indwelling catheters, which provide a direct passage for bacteria or other microorganisms into the bladder. Thus, indwelling catheters can only be used for relatively short-term situations. In addition, indwelling catheters and associated collection bags are not cosmetically appealing to most patients.

An approach that has been taken to address urinary incontinence involves the use of prosthetic urethral valves. One known prosthetic urethral valve utilizes an inflatable cuff that is inserted around the outside of the urethra. Prosthetic urethral valves also have numerous disadvantages. One disadvantage of these valves is that they typically require surgery for installation, and some of these valves must be operated externally and, therefore, are dependent on manual intervention.

The use of intra-urethral valves to address urinary tract disorders is also known. Typical intra-urethral valves also generally require manual intervention. Another problem associated with typical intra-urethral valves is that the valves may be displaced into the bladder or expelled from the urethra. There is also an infection risk associated with many such valves since they often extend into the meatus and/or have portions of the device external to the urethra providing a passage for microorganisms into the bladder.

Electrical stimulation therapy, including rectal, intra-vaginal, and external varieties, has been used to tone the muscles and to stimulate nerves supporting the bladder and urethra. However, this type of therapy requires lengthy and numerous treatments, and any benefits derived from the therapy typically diminish when the treatments are stopped.

Current surgical incontinence procedures typically focus on the augmentation of urethral flow resistance. Such surgical interventions typically include bladder neck suspensions and bulk (collagen) injections. Although these procedures can be clinically effective with certain patients, problems include widely variable clinical outcomes, relatively high costs to perform, and potential complications related to surgery. Moreover, the effects of such surgical procedures may be short-lived.

Drug therapy also exists for a number of urinary tract conditions, including overactive bladder. These drugs include oral medications (systemic) and drugs delivered directly into the bladder. Unfortunately, these drugs typically suffer from side effects, lack of efficacy and high morbidity. In particular, oral medications typically do not provide immediate relief of symptoms and include side effects, such as dry mouth and constipation. Drugs delivered directly into the bladder often require continuous or intermittent catheterization for introduction of the therapeutic agents at the clinically appropriate time.

As can be appreciated, the treatment methods described above either focus on the augmentation of urethral flow resistance, the temporary stoppage or absorption of all urethral flow, or the relaxing of the detrusor muscles to minimize unwanted contractions. The disadvantages and limitations of these treatment methods are numerous and include: an excessively high level of patient interaction required to operate and/or to maintain the devices, especially for elderly patients and for physically or mentally challenged patients; limited clinical efficacy; restricted urine outflow; patient discomfort and side effects; urethral and bladder infections related to the devices used; and relatively great expense as compared to non-clinical solutions (diapers, pads, etc.).

Accordingly, an alternative approach to those described above has been to implant a compressible, pressure-attenuating device in the bladder in order to lower the intravesical pressure. This approach is disclosed, for example, in the following documents, all of which are incorporated herein by reference: U.S. Pat. No. 6,682,473, Matsuura et al., issued Jan. 27, 2004; U.S. Pat. No. 7,074,178, Connors et al., issued Jul. 11, 2006; and U.S. Patent Application Publication No. 2010/0222802, Gillespie, Jr. et al., published Sep. 2, 2010. According to one aspect of the foregoing approach, a compressible device is inserted, in a compacted state, into the bladder of a patient through the patient's urethra, and, then, once in the bladder, the compressible device is expanded, for example, by inflation with atmospheric air. A delivery system may be used to deliver the compressible device through the urethra and into the bladder and also may be used to expand the compressible device from its compacted state to its expanded state and to deploy the compressible device, once expanded, from the delivery system. If removal or replacement of the compressible device is desired, a removal system may be used to remove the compressible device from the bladder through the urethra.

### SUMMARY

Although the above-described implantable, compressible, pressure-attenuating device has had success in treating urinary tract disorders, the present disclosure identifies certain areas of improvement relating to the device, its introduction into a patient, its expansion and deployment within a patient, and its removal from a patient.

It is an object of the disclosure to provide a method and system for performing a medical procedure on an anatomical structure of a body. The medical procedure may be performed, for example, to attenuate transient pressure waves in the anatomical structure and may involve, for example, implanting a compressible pressure-attenuating device in the anatomical structure subject to such pressure waves. Such a method and system may be used in, but is not limited to use in, treating urinary tract disorders.

A system may comprise one or more of the following: an access device, a therapeutic or diagnostic object, a delivery device, and a removal device. The access device may be used to create a passageway to the anatomical structure, such as, for example, a trans-urethral passageway to a patient's bladder. The therapeutic or diagnostic object may be an inflatable device and may be, for example, a pressure-attenuating device. The delivery device may be used to deliver a therapeutic or diagnostic object to the anatomical structure. Such an object may be, for example, a pressure-attenuating device, which may be delivered to the anatomical structure in a compacted or deflated state and then inflated and released from the delivery device. The removal device may be used to view the anatomical structure. In addition, where an object delivered to the anatomical structure is an inflatable pressure-attenuating device, the removal device may also be used to capture, to deflate, and to remove the pressure-attenuating device from the anatomical structure.

In some embodiments, an access device can provide access to an anatomical structure within a patient. The access device can comprise an elongated sheath or cannula or elongate body, the elongated sheath comprising a proximal end, a distal end, and a longitudinal channel. The access device may also include an obturator that can be removably mounted within the longitudinal channel of the elongated sheath.

In some embodiments, an access device can comprise one or more of a housing assembly, a sheath assembly, and a fluid control system. The housing assembly can comprise one or more housing structures that define a body for the access device.

According to one aspect, there is provided an access device for use in providing access to an anatomical structure within a patient. The access device can comprise (a) an elongated sheath or elongated body, the elongated sheath comprising a channel; (b) an obturator, the obturator being insertable into the channel of the elongated sheath; and (c) a locking mechanism for selectively locking the obturator within the channel of the elongated sheath.

According to another aspect, there is provided an access device for use in providing access to an anatomical structure within a patient. The access device can comprise (a) an elongated sheath or elongated body, the elongated sheath comprising a sheath channel; (b) an obturator, the obturator being insertable into the sheath channel of the elongated sheath, the obturator comprising an obturator channel; and (c) an obturator handle, the obturator handle being secured to a proximal end of the obturator, the obturator handle comprising a handle channel, the handle channel being in fluid communication with the obturator channel.

In some embodiments, the access device can include a system for positioning a flexible sleeve in an access channel. The flexible sleeve can be used to protect the access channel and/or body tissue in the patient. For example, in some embodiments, the obturator can include a cavity, and the sleeve can be positionable in the cavity in a first position and positionable outside of the cavity in a second position. The distal end of the obturator may also be positionable distally beyond the distal end of an elongated sheath or elongated body. In some embodiments, a slide ring can be connected to the sleeve to move the sleeve between the first and second positions.

According to another aspect, there can be provided a removal device. The removal device can include at least one manually-actuatable member; and at least one movable arm or jaw, the at least one movable jaw being operable by actuation of the at least one manually-actuatable member.

According to another aspect, there can be provided a removal device. The removal device can comprise (a) at least one manually-actuatable member; (b) at least two jaws, at least one of the at least two jaws being moveable by actuation of the at least one manually-actuatable member; (c) a cystoscope, the cystoscope being positioned to enable observation of the at least two jaws, wherein the cystoscope is a wide angle cystoscope.

According to another aspect, there can be provided a removal device. The removal device can comprise (a) at least one manually-actuatable member; (b) at least two jaws, at least one of the at least two jaws being movable by actuating the at least one manually-actuatable member, wherein at least one of the at least two jaws comprises a gripping member, such as teeth, to securely hold an object to be removed and wherein at least one of the at least two jaws comprises a puncturing member, such as a blade, scissor, pin, hook, or the like, to puncture the object to be removed.

In a first aspect of the disclosure, a removal device can comprise of at least one manually-actuatable member; and at least two opposing deflectable jaws, at least one of the at least two opposing deflectable jaws being movable by actuation of the at least one manually-actuatable member, wherein at least one of the at least two opposing deflectable jaws can comprise a gripping member and wherein at least one of the at least two opposing deflectable jaws can comprise a puncturing member, wherein the at least two opposing deflectable jaws can be configured to deflect vertically.

In some aspects, the removal device can include one or more of the following features in any combination: (a) the puncturing member can comprise a cannulated needle; (b) the at least two opposing deflectable jaws can be configured to open laterally; (c) at least one pull wire can be coupled to the at least one manually-actuatable member and the at least two opposing deflectable jaws; (d) the at least one pull wire can comprise a first pull wire and a second pull wire; (e); the first pull wire can be configured to open the at least two opposing deflectable jaws and the second pull wire can be configured to deflect the at least two opposing deflectable jaws. A single pull wire can be coupled to the at least one manually-actuatable member and the at least two opposing deflectable jaws; (f) the single pull wire being can be configured to both open the at least two opposing deflectable jaws and deflect the at least two opposing deflectable jaws; (g) the single pull wire can include axial section, a curved section and a neck section, the curved section can cause the at least two deflectable jaws to deflect vertically and the neck section can be controlling when the least two opposing deflectable jaws are open; (h) the jaw opposing the jaw with the puncturing member includes an opening configured to receive the cannulated needle when the at least two opposing deflectable jaws can be in a closed position; (i) the at least two opposing deflectable jaws comprises a puncturing member and an opening, the opening of each of the at least two jaws opposing deflectable jaws being adapted to receive the puncturing member on the opposing deflectable jaw when the at least two opposing deflectable jaws can be in a closed position; (j) each puncturing member can comprise a cannulated needle; (k) the jaws can comprise a row of teeth; (l) both of the at least two opposing deflectable jaws can include gripping members; (m) the gripping members can comprise a row of teeth; (n) the closed position can have a gap between the rows of teeth of the at least two opposing deflectable jaws; (o) the puncturing member can be generally perpendicular to the jaw and (p) the device can further comprise a camera.

In another aspect of a disclosure, a removal device can comprise at least one manually-actuatable member; and at least two opposing deflectable jaws, at least one of the at least two opposing deflectable jaws being movable by actuation of the at least one manually-actuatable member, wherein at least one of the at least two opposing deflectable jaws can comprise a gripping member and wherein at least one of the at least two opposing deflectable jaws can comprise a puncturing member, wherein the at least two opposing deflectable jaws can be configured to deflect vertically.

In some aspects, the removal device can include one or more of the following features in any combination (a) the jaw opposing the jaw with the puncturing member can include an opening configured to receive the puncturing member when the at least two opposing deflectable jaws can be in a closed position; (b) device can have at least two opposing deflectable jaws can be configured to open laterally; (c) the device can comprise at least one pull wire coupled to the at least one manually-actuatable member and the at least two opposing deflectable jaws; (d) the at least one pull wire can comprise a first pull wire and a second pull wire; (e) the first pull wire can be configured to open the at least two opposing deflectable jaws and the second pull wire can be configured to deflect the at least two opposing deflectable jaws; (f) the device can have a single pull wire coupled to the at least one manually-actuatable member and the at least two opposing deflectable jaws; (g) the single pull wire can be configured to both open the at least two opposing deflectable jaws and deflect the at least two opposing deflectable jaws; (h) the single pull wire can include axial section, a curved section and a neck section, the curved section can cause the at least two deflectable jaws to deflect vertically and the neck section controlling when the least two opposing deflectable jaws are open; (i) each of the at least two opposing deflectable jaws can comprise a puncturing member and an opening, the opening of each of the at least opposing two deflectable jaws can be adapted to receive the puncturing member on the opposing jaw when the at least two opposing deflectable jaws can be in a closed position; (j) the gripping member can comprise a row of teeth; (k) the device can have both of the at least two opposing deflectable jaws include gripping members; (l) the puncturing member can be a cannulated needle; (m) the gripping members can comprise a row of teeth; (n) the puncturing member can be a cannulated needle; (o) the closed position can have a gap between the rows of teeth of the at least two opposing deflectable jaws; (p) the puncturing member can be a cannulated needle; (q) the puncturing member can be generally perpendicular to the jaw; (r) the puncturing member can be a cannulated needle; and (s) the device can further comprise a camera.

In another aspect of a disclosure, a removal device can comprise an elongate member having a channel therethrough that can be configured to receive a scope, the channel can have a channel opening at a proximal end of the channel and a scope port at a distal end of the channel; a recess adjacent the scope port; at least one actuatable member that can be coupled to a proximal end of the elongate member; and at least two opposing jaws that can be coupled to a distal end of the elongate member, at least one of the at least two opposing deflectable jaws can be movable by actuation of the at least one manually-actuatable member.

In some aspects, the removal device can include one or more of the following features in any combination: (a) the recess can comprise a surface configured to decrease reflection of light from the scope; (b) recess can comprise a surface configured to absorb light from the scope; (c) the recess can comprise a surface configured to deflect light from the scope away from the scope port; (d) the recess can comprises a continuous curved surface; (e) the recess can be formed in a surface that at least partially blocks a view of the scope; (f) the surface that at least partially blocks a view of the scope can comprise at least one of the at least two opposing jaws and a bracket adjacent to the scope port, the bracket can be configured to hold the at least two opposing jaws; (g) the at least two opposing jaws can be configured to both open laterally and deflect vertically. The device can further comprise a camera and (h) the elongate member can have a drain/fill feature.

In another aspect of the disclosure; a removal system can comprise an elongate sheath; the elongate sheath can comprise of a channel extending along the elongate sheath a valve; a seal; and a pressure relief valve. The removal can comprise an elongate member; at least one actuatable member coupled to a proximal end of the elongate member; and at least two opposing jaws coupled to a distal end of the elongate member, at least one of the at least two opposing jaws can be movable by actuation of the at least one manually-actuatable member, wherein the elongate member and the at least two opposing jaws can be configured to pass through at least a portion of the elongate sheath during use such that the elongate member can be within at least one of the elongate sheath, the valve, and the seal. In some examples, the device can further comprise a camera. The elongate member can have a drain/fill feature

In another aspect of the disclosure; removal device can comprise at least one manually-actuatable member; and at least two opposing jaws, at least one of the at least two opposing jaws can be movable by actuation of the at least one manually-actuatable member, wherein at least one of the at least two opposing jaws can comprise at least one puncturing member, at least transverse vent hole, and an external vent channel. In some examples, the device can further comprise a camera.

In another aspect of the disclosure; a removal system can comprise an elongate sheath. The elongate sheath can comprise a channel extending along the elongate sheath; a plurality of vent holes adjacent a distal end of the elongate sheath; a removal device, the removal device can comprise of an elongate member; at least one actuatable member coupled to a proximal end of the elongate member; and at least two opposing jaws, at least one of the at least two opposing jaws can be movable by actuation of the at least one manually-actuatable member, wherein at least one of the at least two opposing jaws can comprise of at least one puncturing member, at least transverse vent hole, and an external vent channel.

In some aspects, the removal system can include one or more of the following features in any combination;(a) the device can further comprise a camera; (b) camera can be attached to the elongate sheath; (c) elongate member can comprises of a camera; (d) elongate member can have a drain/fill feature; and (d) vent holes can be configured to allow air from a deflating implant to escape to minimize a pressure differential.

In some aspects of the disclosure, the sheath for introduction of a device into a bladder can comprise of an elongate body that can be configured to accept the device, the elongate body can comprise a plurality of vent holes adjacent a distal end of the elongate body; a valve that can be configured to prevent flow of a fluid through the elongate body when the device is not present within the elongate body; and a seal that can be configured to prevent flow of a fluid through the elongate body when the device is present within the elongate body. The sheath can further comprise a camera. The vent holes can be configured to allow air from a deflating implant to escape to minimize a pressure differential.

In some aspects of the disclosure; a method of removing an inflatable implant can comprise the steps of inserting an elongate body into a bladder, the elongate body can be configured to accept a removal device, the elongate body can comprise of a plurality of vent holes; inserting the removal device into the bladder by inserting the removal device into the elongate body, with the removal device, grasping and at least partially deflating an inflatable implant in the bladder; and withdrawing the at least partially deflated inflatable implant into the elongate body by withdrawing the removal device into the elongate body, wherein air from the inflatable implant device can escape through the plurality of vent holes. In some examples, the method can further comprise of steps wherein the plurality of vent holes are adjacent a distal end of the elongate body.

In some aspects, the removal device can include one or more of the following features in any combination The method of removing the device further can comprise of an elongate member; at least one actuatable member can be coupled to a proximal end of the elongate member; and at least two opposing jaws, at least one of the at least two opposing jaws being movable by actuation of the at least one manually-actuatable member, wherein at least one of the at least two opposing jaws can comprise of at least one puncturing member, at least transverse vent hole, and an external vent channel.

In some examples, the method can further comprise of steps removing the removal device from the elongate body. The method can further comprises removing the elongate body from the bladder.

### BRIEF DESCRIPTION OF THE DRAWINGS

These and other features, aspects and advantages are described below with reference to the drawings, which are intended to illustrate but not to limit the invention. In the drawings, like reference characters denote corresponding features consistently throughout similar embodiments.

FIG. 1 is a side view of a first embodiment of some of the components of a system for treating a patient;

FIGS. 2(a) through 2(c) are side, partially exploded side, and side-partly in section, views, respectively, of the access device shown in FIG. 1;

FIGS. 3(a) and 3(b) are side and section views, respectively, of the valve assembly shown in FIG. 2(b);

FIG. 4(a) is a side view, partly in section, of the combination of the valve assembly shown in FIGS. 3(a) and 3(b);

FIG. 5(a) shows the combination of the handle and valve assembly of FIG. 4(a), together with an O-ring;

FIGS. 6(a) and 6(b) are section and distal views, respectively, of the seal shown in FIG. 2(b);

FIG. 7(a) and FIG. 7(b) are flowcharts, schematically illustrating methods of implanting the access device of FIGS. 2(a) through 2(c) in a patient;

FIGS. 8(a) through 8(d) are side views, some partly in section and/or broken away in part, illustrating certain steps of the method shown in FIGS. 7(a)-7(b);

FIGS. 9(a) through 9(d) are side, partially exploded fragmentary perspective, fragmentary top, and fragmentary top, broken away in part, views, respectively, of the removal device shown in FIG. 1;

FIGS. 10 and 11 are respective section views of the scope connector and ring shown in FIG. 9(a);

FIGS. 12, 13, and 14 are respective fragmentary section views of the scope guide, cystoscope, and support shown in FIG. 9(a).

FIG. 15 is a section view of the bracket shown in FIG. 9(a);

FIG. 16 is a fragmentary section view of the rod shown in FIG. 9(a);

FIG. 17 is a section view of the connector shown in FIG. 9(b);

FIG. 18 is a section view of the linking arm shown in FIG. 9(b);

FIG. 19 is a section view of the linking arm shown in FIG. 9(b);

FIGS. 20(a) through 20(d) are left side, right side, top, and section views, respectively, of one of the jaws shown in FIG. 9(b); FIGS. 20(e) and 20(f) are right and top section views of an alternative design of the jaws shown in FIG. 9(b).

FIGS. 21(a) through 21(d) are left side, right side, top, and section views, respectively, of the other jaw shown in FIG. 9(b).

FIG. 22 is an embodiment of a removal device.

FIG. 23 is an exploded view of the removal device of FIG. 22.

FIGS. 24(a)-(o) are various portions of the removal device shown exploded in FIG. 23. FIG. 24(a) shows various views of an embodiment of a distal tip. FIG. 24(b) shows various views of an embodiment of a proximal hub. FIG. 24(c) illustrates an embodiment of a proximal hub accepting the proximal end of an embodiment of an outer tube and accepting the distal end of an embodiment of a proximal tube. FIG. 24(d) illustrates various views of an embodiment of a nut. FIG. 24(e) illustrates various views of an embodiment of a retaining block. FIG. 24(f) illustrates various views of an embodiment of a moving handle. FIG. 24(g) illustrates various views of an embodiment of a jaw assembly connected to a pull wire. FIG. 24(h) illustrates various views of an embodiment of a pull wire linkage. FIG. 24(i) illustrates various views of an embodiment of a link. FIG. 24(j) shows an embodiment with a drain/fill feature 2300. In some embodiments, the drain/fill feature 2300 can be used to drain or fill the bladder. FIG. 24(k) shows an axial view of an embodiment where the outer tube has a flattened surface acting as a drain/fill feature. FIG. 24(l) shows an axial view of an embodiment where the outer tube has an impression acting as a drain/fill feature. FIG. 24(l) shows an axial view of an embodiment where the outer tube has a ridged feature acting as a drain/fill feature. FIG. 24(n) shows an embodiment that uses a camera attached to the removal device. FIG. 24(o) shows an embodiment where the camera and camera wire are fed through the removal device itself. FIG. 24(p) and FIG. 24(q) show embodiments that use a camera attached to the sheath of the removal device.

FIGS. 25 & 26 show an embodiment of a removal device having deflectable jaws.

FIG. 27 is an exploded view of the removal device of FIGS. 25-26.

FIG. 28 shows the removal device of FIGS. 25-26 being used with a scope.

FIGS. 29-31 show an embodiment of the distal end of a removal device having deflectable jaws.

FIG. 32 shows an embodiment of opposing jaws having venting features.

FIG. 33(a)-(f) show various different embodiments of opposing jaws.

FIG. 34(a) is an embodiment of the distal end of a removal device having deflectable jaws.

FIGS. 34(b)-(d) are various portion of an embodiment of a removal device having deflectable jaws. FIG. 34(b) shows various view of an embodiment of a distal tip proximal portion. FIG. 34(c) illustrates an embodiment of a proximal hub accepting the proximal end of an embodiment of an outer tube and accepting the distal end of an embodiment of a proximal tube. FIG. 34(d) illustrates various views of an embodiment of a distal tip distal portion.

FIG. 35 illustrates an embodiment of a jaw assembly coupled to pull wires.

FIGS. 36(a)-(c) and 37(a)-(c) show various pull wire and pull wire connector configurations.

FIGS. 38 and 38(a)-(c) are various views of an embodiment of a removal device in different stages of actuation.

FIGS. 39(a)-(c) are various views of an embodiment of the handle of a removal device handle in different stages of actuation.

FIGS. 40(a)-(d) shows various relationships between handle actuation and jaw position for various embodiments of a removal device.

FIGS. 41(a)-(c) show various embodiments of a pull wire.

FIG. 41(d) shows a cross-sectional view of a pull wire within a distal portion of an embodiment of a removal device.

FIG. 42 is another embodiment of a removal device having deflectable jaws.

FIGS. 43-44 are embodiments of a removal device handle that may be used with removal devices having more than one pull wire.

FIGS. 45-47 are embodiments of removal device jaws having a fixed jaw and an actuatable jaw.

FIG. 48 is a flowchart, schematically illustrating one method of using the removal device of FIG. 9(a) to remove the pressure-attenuating device from a patient;

FIGS. 49(a) through 49(d) are fragmentary side views, partly in section, illustrating certain parts of steps of the method shown in FIG. 48;

FIGS. 50(a) and 50(b) are fragmentary top views of a first alternate embodiment to the removal device shown in FIG. 1, the jaws of the removal device being shown in a closed state and in an open state, respectively;

FIG. 51 is a fragmentary section view of the removal device of FIGS. 50(a) and 50(b);

FIGS. 52(a) and 52(b) are fragmentary top views of a second alternate embodiment to the removal device shown in FIG. 1, the jaws of the removal device being shown in a closed state and in an open state, respectively;

FIG. 52(c) is a fragmentary section view of the removal device of FIGS. 52(a) and 52(b);

FIG. 53 is a side view of a third alternate embodiment to the removal device shown in FIG. 1, the removal device being shown in an open state;

FIG. 54 is an enlarged fragmentary side view of a proximal portion of the removal device shown in FIG. 53;

FIGS. 55(a) through 55(c) are enlarged fragmentary side, top, and perspective views, respectively, of a distal portion of the removal device shown in FIG. 53;

FIGS. 56(a) through 56(c) are side, perspective, and exploded views, respectively, of a fourth alternate embodiment to the removal device shown in FIG. 1, the removal device being shown with the two jaws in a closed state;

FIG. 57 is a side view of the removal device shown in FIGS. 56(a) through 56(c), the removal device being shown with the two jaws in an open state;

FIG. 58 is an enlarged fragmentary exploded view of a distal portion of the removal device shown in FIGS. 56(a) through 56(c), the two jaws being shown in a closed state, the cystoscope and the wires not being shown;

FIGS. 59(a) and 59(b) are enlarged fragmentary bottom and top views, respectively, of a distal portion of the removal device shown in FIGS. 56(a) through 56(c), the two jaws of the removal device being shown in a closed state, the cystoscope not being shown;

FIGS. 60(a) and 60(b) are enlarged fragmentary top and perspective views, respectively, of the distal portion of the removal device shown in FIGS. 56(a) through 56(c), the two jaws of the removal device being shown in an open state, the cystoscope not being shown;

FIG. 61 is an enlarged side view of the handle assembly shown in FIGS. 56(a) through 56(c);

FIGS. 62(a), 63(a), and 64(a) are enlarged side, top, and perspective views, respectively, of the jaw assembly shown in FIGS. 56(a) through 56(c), the two jaws of the jaw assembly shown in a closed state;

FIGS. 65(a) and 65(b) are enlarged top and perspective views, respectively, of the jaw assembly shown in FIG. 57, the two jaws shown in an open state;

FIGS. 66(a) and 66(b) are enlarged end and side views, respectively, of the tube body shown in FIGS. 56(a) through 56(c);

FIGS. 67(a) and 67(b) are side and top views, respectively, of the wire shown in FIGS. 56(a) through 56(c);

FIG. 68 is an enlarged fragmentary perspective view of the distal end of the wire shown in FIGS. 66(a) and 66(b);

FIG. 69 is an enlarged fragmentary side view of the proximal end of the wire shown in FIGS. 66(a) and 66(b); and

FIGS. 70(a) and 70(b) are enlarged side and perspective views, respectively, of one of the needles shown in FIG. 56(c).

FIG. 71 is an embodiment of a sheath that may be used to introduce various devices into an anatomic cavity.

FIG. 72 is an exploded view of the sheath shown in FIG. 71.

FIG. 73(a) is an embodiment of a seal and valve that may be used in connection with the sheath shown in FIG. 71. FIGS. 73(b)-73(c) illustrates embodiments of a seal and valve incorporating a pressure release valve.

FIG. 74 is another view of the sheath shown in FIG. 71.

FIGS. 75(a)-75(b) shows an embodiment of a sheath and an obturator device that may be used to initially introduce the sheath into an anatomic cavity. FIG. 75(b) shows the obturator placed inside of the sheath.

FIG. 76(a) shows an embodiment of a sheath and a visual obturator device that may be introduced into an anatomic cavity using the sheath. FIG. 76(b) shows the visual obturator inserted into the sheath.

FIGS. 77-78 show embodiments of a blunt nose obturator and a visual obturator, respectively.

FIG. 79 shows an embodiment of a removal device.

FIGS. 80(a)-80(h) shows a method of how to remove an inflatable device.

FIGS. 81 and 82 are fragmentary side views, partly in section, illustrating a removal device inserted through a sheath being used to remove an inflatable implant from within a bladder.

### DETAILED DESCRIPTION

Medical devices, methods, and systems related thereto for use within the body are disclosed. The medical devices and medical systems can include pressurized therapeutic devices, implants, implant delivery devices, implant retrieval devices, expandable or compressible membrane enclosures or balloons, sponges, foams, attenuators, space occupying members, and space creating devices, and therapeutic devices. Though urology and use in the bladder will be primarily discussed, it will be understood that the systems and methods can be used elsewhere. The medical devices and medical systems can be used for many purposes and in many places within the body including, but not limited to, the following systems of the human body: cardiovascular, pulmonary, renal/urological, gastrointestinal, hepatic/biliary, gynecological, neurological, musculoskeletal, otorhinolaryngological and ophthalmic, as well as in and around organs of the body, and in intra- and inter-organ spaces.

In one particular aspect, the disclosure relates generally to the field of urology, and in particular to the treatment of disorders of the urinary tract caused by sudden fluctuations of intravesical pressure. More specifically, in this aspect, methods, systems, and devices are provided for the treatment of urinary disorders, such as incontinence, urgency, frequency, interstitial cystitis, irritable bladder syndrome, and neurogenic bladders.

Some embodiments provide methods, systems, and devices for treating and/or compensating for reduced dynamic compliance of the bladder. In one embodiment, a device having a compressible element is placed within the human urinary bladder in a manner that allows the compressible element to act as a pressure attenuator to attenuate transient pressure events. The term “attenuator” refers generally to devices that attenuate pressure, force, or energy by dissipating or dampening the pressure, force, or energy. Gases, such as atmospheric air, carbon dioxide, nitrogen, and certain perfluorocarbons (PFC), are very compressible in the pressure ranges typically encountered in the human bladder and may be used in attenuation devices inserted in the bladder. Furthermore, when compared to the tissues encompassing liquid, gases are significantly more compliant than the immediate environment. The addition of a volume of gas can act as a low or variable rate spring in series with the native fluidic circuit of the urinary tract.

In accordance with one embodiment, an attenuation device is placed within the human urinary bladder. The attenuation device can be a pressurized container with a positive or negative pressure. The container can take many forms including a sphere. The attenuation device may be untethered in the bladder and may remain in the bladder for between several hours and one year. The attenuation device can be a small elastomeric gas cell with a relaxed (unstretched) volume of between about 0.1 and 500 cc, more preferably between about 1 and 180 cc, and more preferably still, between about 10 and 60 cc. The attenuation device can be a unitary component or can comprise two or more subcomponents. The attenuation device can be made with a seam or without a seam but preferably is made without a seam. The attenuation device can have a substantially uniform wall thickness of between about 0.25 inch to 0.0001 inch, more preferably between 0.0001 inch and 0.005 inch, but could vary greatly in wall thickness and still perform the intended function.

In the embodiment described above, attenuation devices having gas cells that are free-floating in the bladder have been described. In other embodiments, gas cells or similar attenuation devices could be surgically affixed to the bladder wall through the use of suture, staples or other accepted methods or could be placed submucosally or intramuscularly within the bladder wall. Some embodiments could induce endothelial encapsulation. Other embodiments could also include attenuation devices with programmable, variable and adjustable buoyancy by using ballasting, specific inflation/deflation solutions, alternative materials of construction or by other means.

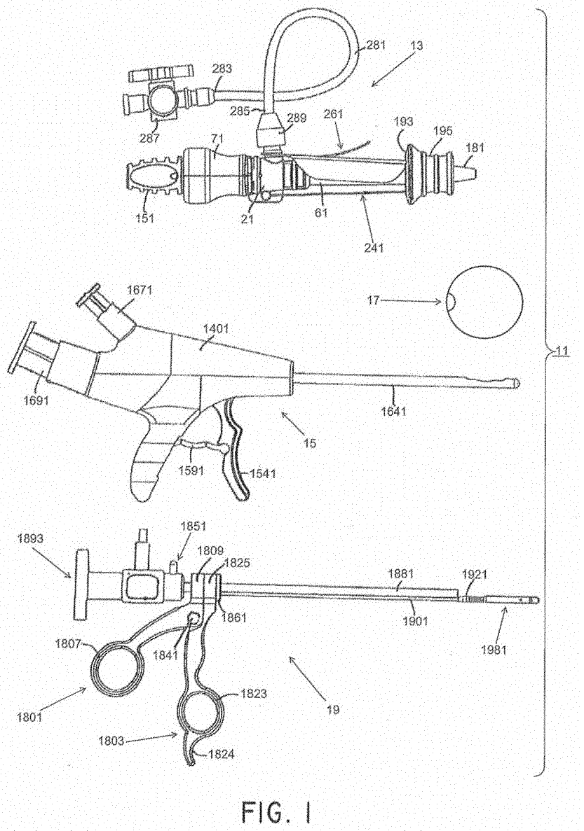

Referring now to FIG. 1, there is shown a side view of components of a first embodiment of a system for treating a patient, the treatment system being represented generally by reference numeral 11. (For ease of illustration and understanding, certain aspects of system 11 may not be shown in FIG. 1.)

System 11 may comprise an access device 13, a delivery device 15, a pressure-attenuating device 17, and a removal device 19. The access device may be used to create a trans-urethral passageway to a patient's bladder. The delivery device may be inserted through the passageway created by the access device and may be used to deliver the pressure-attenuating device to the bladder in a compacted state, then may be used to inflate the pressure-attenuating device, and then may be used to release the inflated, pressure-attenuating device. The removal device may be inserted through the passageway created by the access device and may be used to view the bladder and/or to capture, to deflate and to remove the pressure-attenuating device.

Each of access device 13, delivery device 15, and pressure-attenuating device 17 may be a single-use (i.e., disposable) device or a multiple-use (i.e., reusable) device, but each is preferably a single-use device. Removal device 19 may be a single-use device or a multiple-use device, but preferably is a multiple-use device.

### Access Device

As has been mentioned, an access device may be used to create a passageway into the body. For example, the passageway can be a trans-urethral passageway to a patient's bladder. The access device may be used to drain fluid from the body, such as from the bladder. The access device can be used to protect tissue between the access entry location and the exit location within the body. The access device may further be used as a positioning device to properly position other tools, such as the delivery device within the body. For example, the access device can include a meatal stop, to properly position portions of the delivery device within the bladder.

An access device may include one or more of a housing assembly, a sheath assembly, and a fluid control system. A housing assembly can comprise one or more housing structures that define a body of the access device.

A sheath assembly can comprise an elongated sheath or cannula or elongated body, and a longitudinal channel extending therethrough. In some embodiments, as will be discussed more fully below, the sheath assembly may include a slide ring assembly that is slidably mounted around the sheath, and it may include a protective sleeve. The slide ring assembly can be moved between a distal position and a proximal position. In some embodiments the slide ring assembly can have one or more mechanisms to secure the slide ring assembly in the distal position and/or the proximal position, and positions therebetween. The protective sleeve can be coupled to the slide ring assembly. In some embodiments the access device can include an obturator that can be removably mounted within the longitudinal channel of the sheath.

A fluid control system can control fluid communication between the anatomical structure within the patient and the access device. For example, the fluid control system can be used to drain the bladder of a patient. The fluid control system can have one or more fluid conduits in fluid communication with the sheath. The fluid conduits can be used to remove and/or deliver fluid to/from the patient. The fluid control system can have one or more mechanisms to control the rate of fluid transfer through the access device. In some embodiments the fluid control system can provide a fully open fluid conduit or a fully closed fluid conduit. In some embodiments, the fluid control system can have a mechanism to provide a variable flow rate for each fluid conduit. In some embodiments the flow rate of each fluid conduit can be controlled individually.

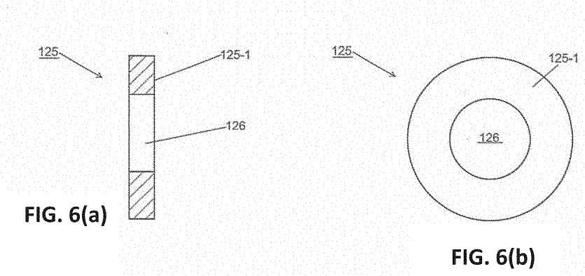

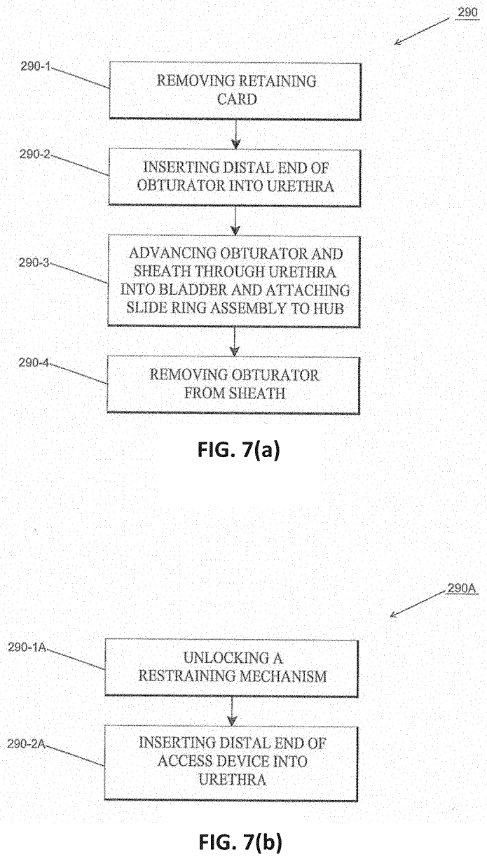

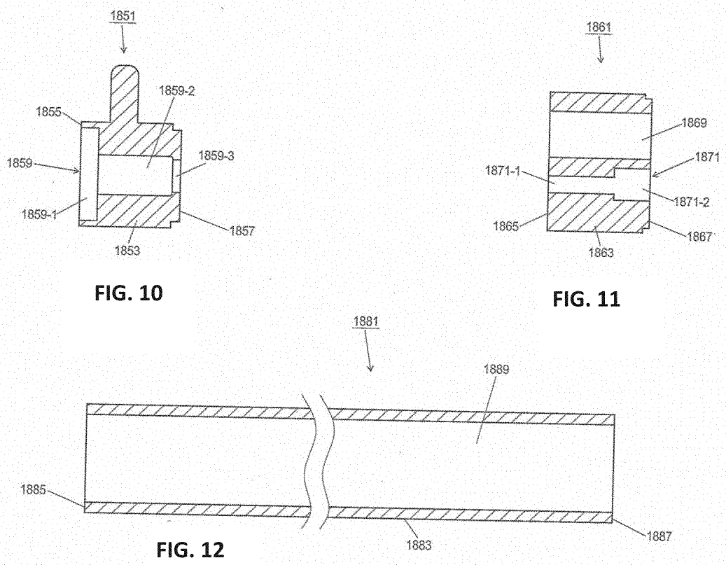

Additional embodiments of access devices are described in U.S. Patent Application Publication No. 2010/0222802, incorporated herein by reference, and referring to cannulas, sheaths, tubular bodies, and/or tubular hubs, meatal stop surface, etc., often as part of a delivery system. Embodiments of an access device, often as part of a delivery system, are also provided in U.S. Pat. No. 6,976,950, incorporated by reference herein. See for example: FIGS. 6-11(a), 34(a)-35(b) and 48(a)-48(d), and the accompanying discussion, including at columns 13-16, and 35. Additional embodiments of access devices, delivery devices, and removal devices are described in U.S. Pat. No. 9,801,658, incorporated herein by reference.

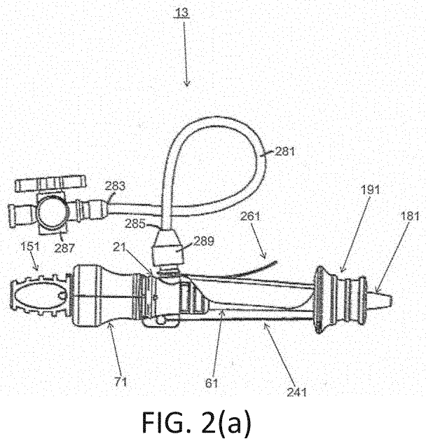

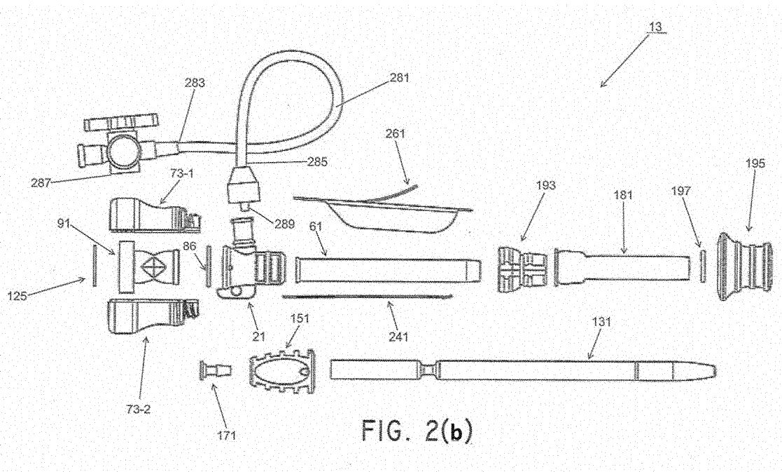

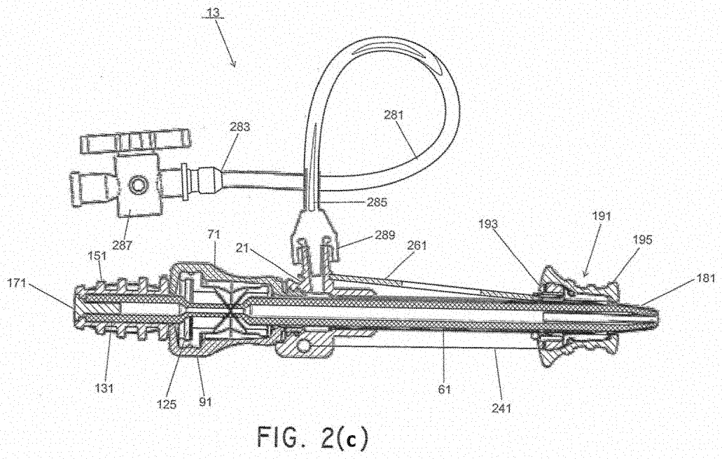

As shown in FIGS. 2(a) through 2(c), access device 13 may include a housing assembly, a sheath assembly, and a fluid control system. The housing assembly may comprise a handle 71. The sheath assembly may comprise a cannula or sheath 61, a dilator or obturator 131, an obturator handle 151, a handle plug 171, a protective sleeve 181, a slide ring assembly 191, and one or more restraining mechanisms 241 and 261. The fluid control system may comprise one or more of a hub 21, a valve assembly 91, a seal 125, and a fluid extension line 281. In some embodiments, the access device 13 may simply comprise a cannula, but may also include a valve assembly and an obturator. It will be understood that other combinations of components could also be used. Each of the components will now be discussed in detail.

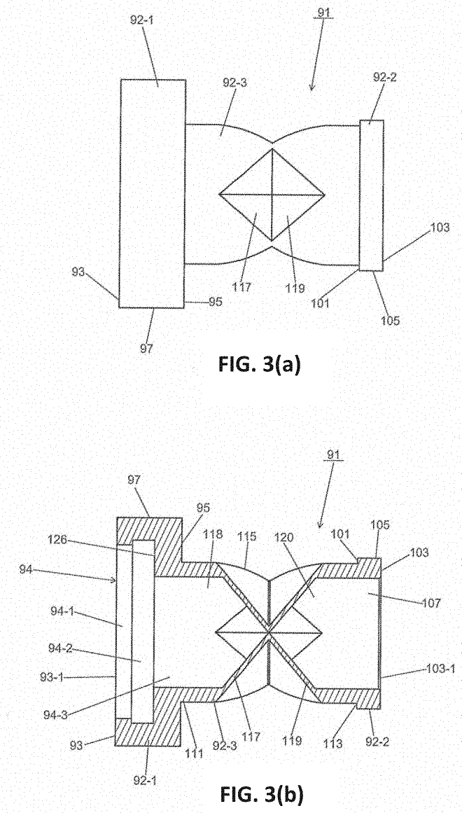

Access device 13 may further comprise a valve assembly 91 (see FIGS. 2(b)-2(c)), which may be disposed within handle 71. Any of a number of different valve assemblies can be used. Valve assembly 91, shown in FIGS. 3(a) and 3(b), may comprise a unitary structure, preferably made of a medical-grade silicone or a similarly suitable material. Valve assembly 91 may be shaped to include a proximal portion 92-1, a distal portion 92-2, and an intermediate portion 92-3. Proximal portion 92-1 may be a generally tubular structure shaped to include a proximal end 93, a distal end 95, and a circular side wall 97. Proximal end 93 may be shaped to include a central opening 93-1 leading to a longitudinal channel 94 extending from proximal end 93 to distal end 95. Channel 94 may include a proximal section 94-1, an intermediate section 94-2, and a distal section 94-3, with intermediate section 94-2 having a comparatively greater diameter, with distal section 94-3 having a comparatively lesser diameter, and with proximal section 94-1 having an intermediate diameter. Distal portion 92-2 of valve assembly 91 may be a generally tubular structure shaped to include a proximal end 101, a distal end 103, and a circular side wall 105. Distal end 103 may have a central opening 103-1 leading to a longitudinal channel 107 extending from proximal end 101 to distal end 103. Intermediate portion 92-3 of valve assembly 91 may be a generally tubular structure shaped to include a proximal end 111, a distal end 113, and a side wall 115. Side wall 115 may be appropriately shaped to define a proximal valve 117 and a distal valve 119. Valves 117 and 119 may divide the interior of intermediate portion 92-3 into a proximal channel 118 that is in fluid communication with distal section 94-3 of proximal portion 92-1 and a distal channel 120 that is in fluid communication with channel 107 of distal portion 92-2. Valves 117 and 119, each of which may be a four-sided duckbill valve, may be oriented in opposite directions relative to one another, with valve 117 tapering in a distal direction and with valve 119 tapering in a proximal direction. Moreover, the distal end of valve 117 and the proximal end of valve 119 may be conjoined so that valves 117 and 119 open and close in unison. As will be discussed further below, valves 117 and 119 may be constructed so as to be biased towards a closed state. While in such a closed state, valves 117 and 119 may serve to prevent fluids or other matter from passing through valve assembly 91. In particular, because of its distally-tapered orientation, valve 117 may serve to prevent fluid from flowing proximally through valve assembly 91. In addition, as will also be discussed further below, valves 117 and 119 may be opened, when desired, by inserting an appropriate medical device through valve assembly 91. A benefit of the opposed orientation of valve 119 relative to valve 117 is that valve 119 may reduce the likelihood that, as a medical device that has previously been inserted through valve assembly 91 is thereafter withdrawn from valve assembly 91, valve 117 will scrape against the exterior of the medical device being withdrawn. Such scraping may be undesirable, for example, where the medical device is a removal device used to remove a pressure-attenuating device from a patient and the scraping causes the pressure-attenuating device to become detached from the removal device.

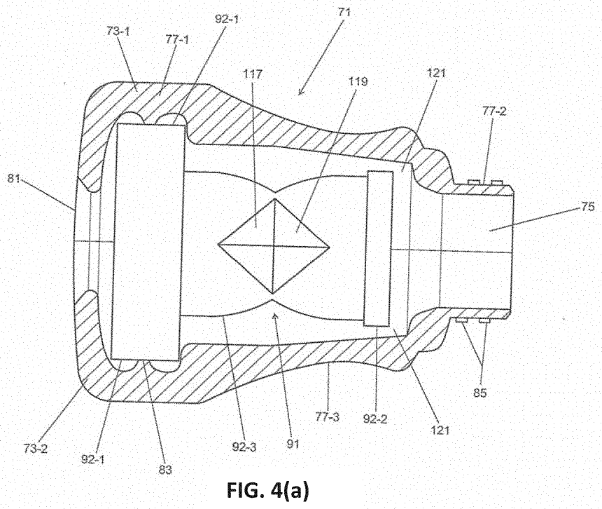

Referring now to FIG. 4(a), there is shown a side view, partly in cross-section, of the combined handle 71 and valve assembly 91. As can be seen, distal portion 92-2 of valve assembly 91 may be dimensioned relative to the interior surface of intermediate portion 77-3 of handle 71 such that a gap 121 may be provided therebetween. Without wishing to be limited to any particular theory of operation, the present inventors believe that gap 121 may be advantageous in permitting a portion of the fluid entering handle 71 through distal portion 77-2 to flow proximally around distal portion 92-2 and to accumulate around the exterior of intermediate portion 92-3 of valve 91. Such accumulated fluid may serve to equalize the fluid pressures within intermediate portion 92-3 of valve assembly 91 and around the exterior of intermediate portion 92-3 of valve assembly 91, thereby promoting the biasing of valves 117 and 119 to a normally closed state. According to one embodiment, gap 121 may be sized to be approximately 0.0001-2 inches, preferably about 0.001-0.500 inch, more preferably about 0.010-0.050 inch.

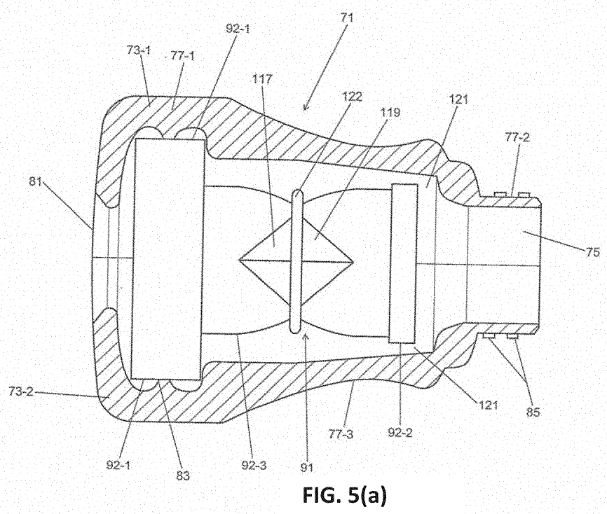

As can also be seen in FIG. 4(a), proximal portion 92-1 of valve assembly 91 may be dimensioned relative to proximal portion 77-1 of handle 71 such that proximal portion 92-1 may form a fluid-tight seal with ridge 83. In this manner, fluid flowing proximally through gap 121 may be kept from flowing proximally past ridge 83. As can be seen in FIG. 5(a), a structure, such as an O-ring 122 may be added around valve assembly 91 to help valves 117 and 119 coapt without relying on cavity pressure. In some embodiments, the valve assembly 91 can be made together with or as part of the handle 71.

Referring now to FIGS. 2(b)-2(c) and 6(a)-6(b), access device 13 may further comprise a seal 125. Seal 125 may comprise a unitary structure, which may be made of a medical-grade silicone or a similarly suitable material. Seal 125 may be of annular shape having a central opening 126. Seal 125 may be appropriately dimensioned to be mounted within valve assembly 91 as shown in FIG. 2(c). Seal 125 may be positioned in valve assembly 91 in intermediate section 94-2 of channel 94, with a rear surface 125-1 of seal 125 fixed by suitable means (e.g., ultrasonic welding, adhesive, etc.) to a shelf 126 (see FIG. 3(b)) within valve assembly 91. Central opening 126 may be appropriately dimensioned to form a fluid-tight seal coaxially around a medical device (e.g., delivery device 15, removal device 19, etc.) that has been inserted through valve assembly 91. In this manner, if valves 117 and 119 are opened by such a medical device inserted through valve assembly 91, seal 125 may serve to minimize the proximal leakage of fluid around said medical device.

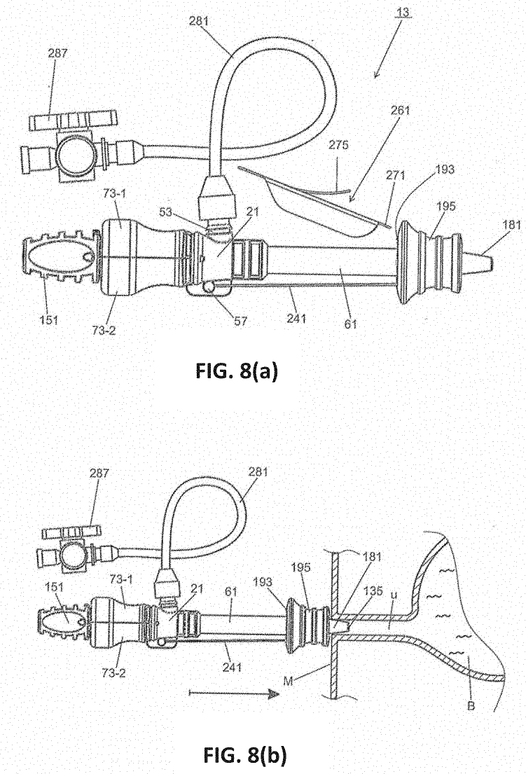

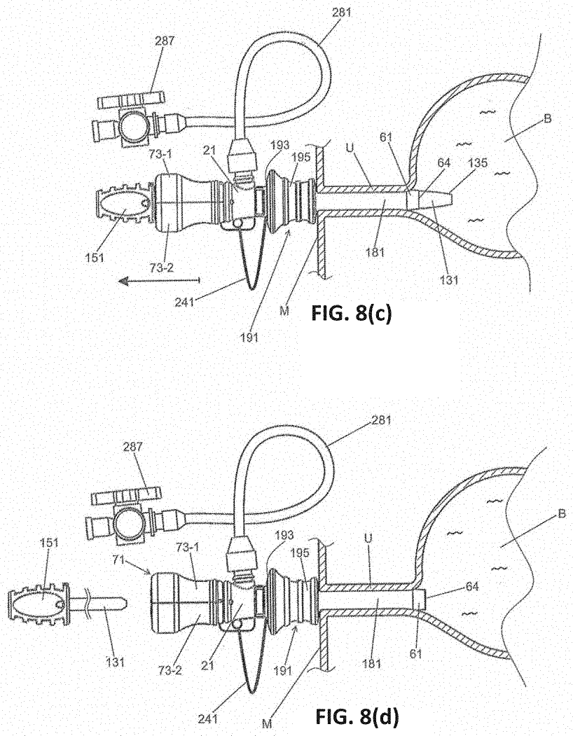

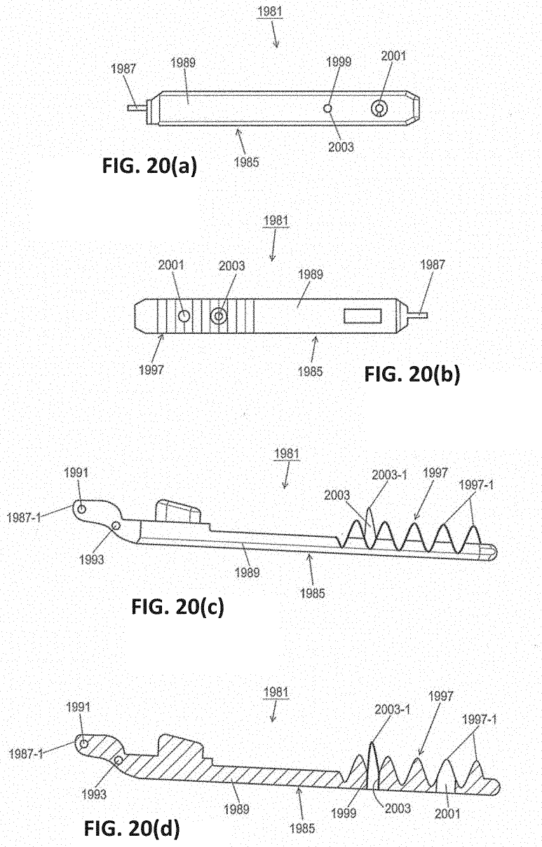

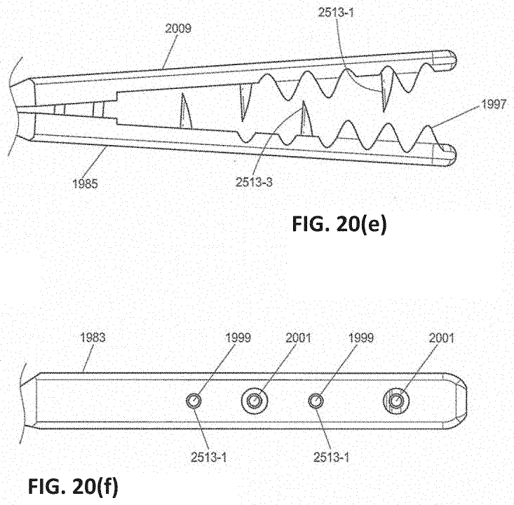

Referring now to FIGS. 20A-B, there are shown two flowcharts, schematically depicting possible methods 290A and 290, respectively, of using access device 13 to provide access to a desired anatomical structure. Such access can be, for example, trans-urethral access to a female human urinary bladder. Method 290A may begin with a step 290-1A of unlocking a restraining mechanism. This can be done, for example, by removing card 261 from device 13, preferably by pulling tab 275 distally until waist 53 of hub 21 disengages from recess 269 of tab 275 and then by removing tab 271 from inner member 193 of slide ring assembly 191 (see FIG. 8(a)). Method 290A may then continue with a step 290-2A of aligning and inserting the distal end of the access device into a body. This can include positioning a meatal stop next to the meatus. This may also include aligning and inserting distal end 135 of obturator 131 into the outer opening of the urethra, with the distal end of slide ring assembly 191 contacting the meatus of the patient and with distal end 135 of obturator 131 being covered by sleeve 181 (see FIG. 8(b) with the urethra being represented by the reference letter U, the meatus being represented by the reference letter M, and the bladder being represented by the reference letter B). Though illustrated schematically, in some embodiments, the slide ring assembly 191 can engage the body tissue at the meatus. Method 290 may then continue with a step 290-3 of advancing obturator 131 and sheath 61 distally through the urethra U in a straight and steady motion until sleeve 181 everts completely (is pulled outward and turned inside out) and slide ring assembly 191 snaps onto the distal end of hub 21 (see FIG. 8(c)). (With obturator 131 and sheath 61 advanced in the manner discussed above, distal end 135 of obturator 131 and distal end 64 of sheath 61 may be positioned within the bladder B of a patient.) Preferably, as obturator 131 and sheath 61 are advanced distally in the manner discussed above, rotation of obturator 131 and sheath 61 relative to slide ring assembly 191 is avoided so as to minimize twisting of sleeve 181, which twisting may impede eversion of sleeve 181.

Method 290 may then proceed to step 290-4 of withdrawing obturator 131 proximally from sheath 61, hub 21, and handle 71 by holding hub 21 stationary with one hand while grasping and pulling on obturator handle 151 with the other hand (see FIG. 8(d)). With obturator 131 thus removed, the remaining implanted portion of access device 13 may provide a conduit through which medical devices, such as delivery device 15, pressure-attenuating device 17, and removal device 19, may be delivered to a desired anatomical structure. During the above-recited steps, stopcock valve 287 may be either opened or closed, depending upon the design of the access device and whether or not one wishes to allow fluid from the patient's bladder to empty. In some embodiments, the obturator 131 can block access to the fluid extension line 281 and the stopcock valve 287. Thus, fluid may be drained after the access sheath is positioned and the obturator 131 removed.

As has been mentioned, other access devices or systems can be used. The access sheath can vary from a basic cannula to any number of different combinations involving at least some of the access sheath components described herein.

As noted above, it may be desirable to minimize the rotation of slide ring assembly 191 relative to obturator 131 and sheath 61 so as to minimize the twisting of sleeve 181 within obturator 131. Although card 261 may satisfactorily prevent such rotation prior to its removal from access device 13, once card 261 has been removed from access device 13, there may be no remaining mechanism in access device 13 for restraining such rotation. Therefore, according to one aspect, certain alternate embodiments are disclosed below that may include a rotation-restraining mechanism.

### Removal

A removal device may be inserted through the passageway created by an access device. The removal device may be used to capture, to deflate and/or to remove the pressure-attenuating device. The removal device may also be used to view the inside of the anatomical structure, as well as the pressure-attenuating device. This viewing may be done during all or part of the capturing, deflating, and/or removing the pressure-attenuating device.

Certain additional embodiments of a removal device are described in U.S. Patent Application Publication No. 2010/0222802, incorporated by reference herein. See for example: FIGS. 20A-22B, 23H, and 24-29C and the accompanying discussion.

Embodiments of a removal device are also provided in U.S. Pat. No. 6,976,950, incorporated by reference herein. See for example: FIGS. 12, and 20-23, and the accompanying discussion, including at columns 18-21, and 25-26. Further embodiments of a removal device are described in U.S. Pat. No. 8,992,412, incorporated by reference herein.

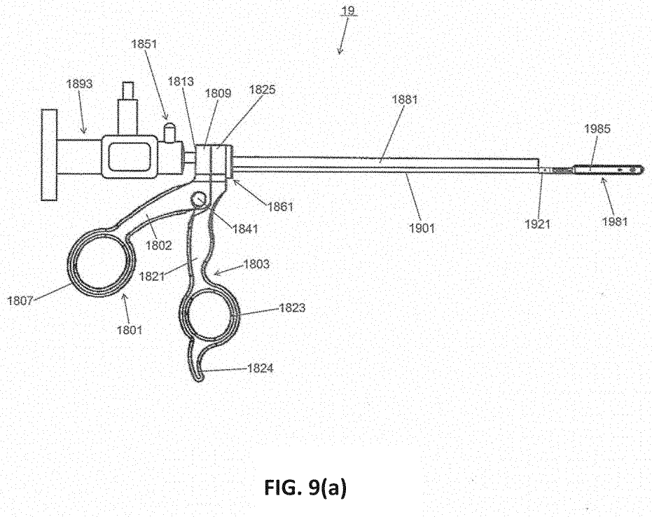

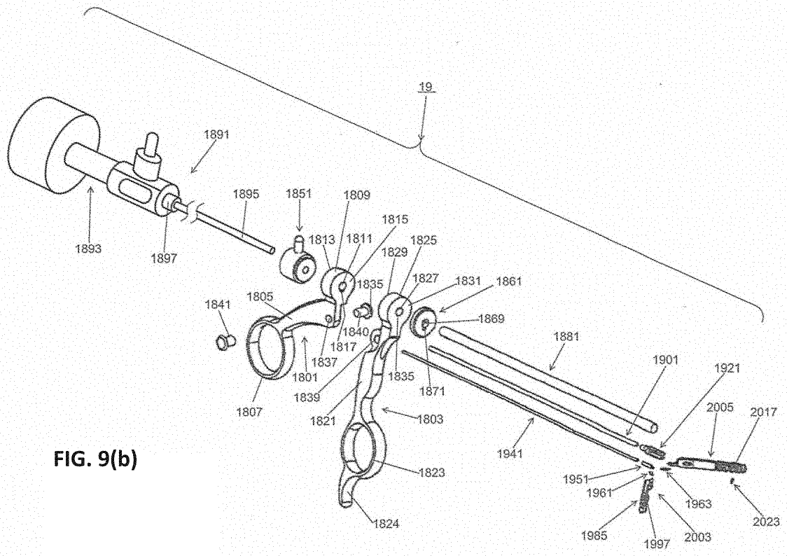

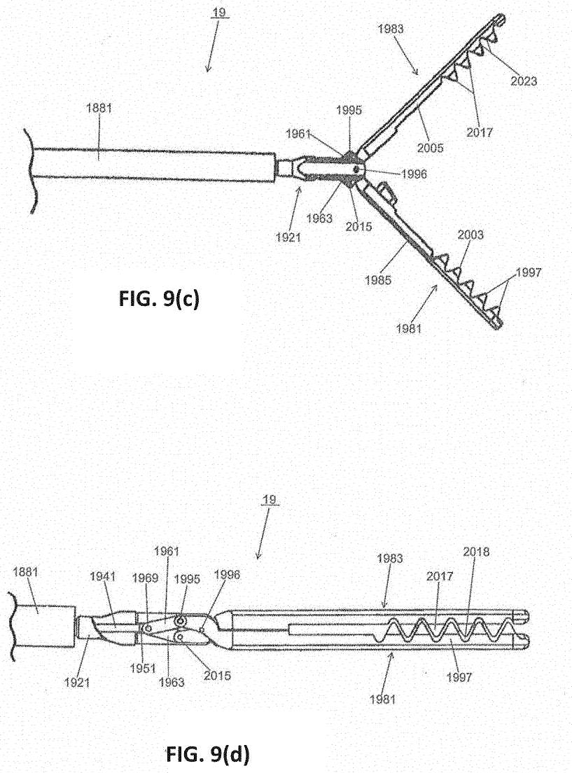

Referring now to FIGS. 9(a) through 9(d), removal device 19 according to certain embodiments is shown. The removal device 19 can include a pair of scissor-like handles, first member 1801 and second member 1803, that can be used to articulate a pair of jaws 1981 and 1983 as will be described below.

First member 1801 may be a unitary structure, preferably made of a hard, medical-grade polymer, polytetrafluoroethylene (PTFE)-coated (TEFLON®) aluminum, or a similarly suitable material. Member 1801 may be shaped to comprise an elongated arm portion 1805 having a transversely-extending ring portion 1807 disposed at one end thereof and having a longitudinally-extending, generally cylindrical portion 1809 disposed at the opposite end thereof. Ring portion 1807 may be appropriately dimensioned to receive a thumb of a user. Cylindrical portion 1809 may be shaped to include a bore 1811 extending longitudinally all the way from a proximal end 1813 to a distal end 1815 and may also be shaped to include a cavity 1817 extending longitudinally for a portion of the distance, but not entirely, from distal end 1815 towards proximal end 1813. Bore 1811 may be of comparatively greater diameter and cavity 1817 may be of comparatively lesser diameter.

Second member 1803 may be a unitary structure, preferably made of a hard, medical-grade polymer, polytetrafluoroethylene (PTFE)-coated (TEFLON®) aluminum, or a similarly suitable material. Member 1803 may be shaped to comprise an elongated arm portion 1821 having both a transversely-extending ring portion 1823 and a finger rest 1824 disposed at one end thereof and having a longitudinally-extending, generally cylindrical portion 1825 disposed at the opposite end thereof. Ring portion 1823 may be appropriately dimensioned to receive a finger of a user, such as a forefinger, and finger rest 1824 may be appropriately dimensioned to receive a finger of a user, such as the middle finger. Cylindrical portion 1825 may be shaped to include a bore 1827 of comparatively greater diameter extending longitudinally all the way from a proximal end 1829 to a distal end 1831 and a bore 1833 of comparatively lesser diameter extending longitudinally all the way from proximal end 1829 to distal end 1831. Bore 1827 and bore 1833 may have their axes generally aligned with bore 1811 and cavity 1817, respectively.

First member 1801 may be coupled to second member 1803 for pivotal movement relative thereto by a pin 1835 inserted through transverse openings 1837 and 1839 in first member 1801 and second member 1803, respectively. Pin 1835 may be held in openings 1837 and 1839 by having an end 1840 received within a cap 1841. In the above manner, first member 1801 may be regarded as a movable member pivotally mounted about pin 1835, and second member 1803 may be regarded as a stationary member.

Removal device 19 may further comprise a scope connector 1851. Connector 1851, which is also shown separately in FIG. 11, may be a unitary structure, preferably made of a medical-grade stainless steel or a similarly suitable material. Connector 1851 may be a generally tubular member comprising a generally circular side wall 1853 defining a proximal end 1855, a distal end 1857, and a longitudinal channel 1859 extending all the way from proximal end 1855 to distal end 1857. Longitudinal channel 1859 may include a proximal portion 1859-1 of comparatively greater diameter, a distal portion 1859-2 of comparatively lesser diameter, and an intermediate portion 1859-3 intermediate in diameter to proximal portion 1859-1 and distal portion 1859-2.

Removal device 19 may further comprise a ring 1861. Ring 1861, which is also shown separately in FIG. 11, may be a unitary structure, preferably made of a medical-grade stainless steel or a similarly suitable material. Ring 1861, which may be fixedly coupled to member 1803, may be a generally tubular member comprising a generally circular side wall 1863 defining a proximal end 1865, a distal end 1867, and a pair of longitudinal bores 1869 and 1871, each of bores 1869 and 1871 extending all the way from proximal end 1865 to distal end 1867. Bore 1869 may be generally aligned with and comparable in diameter to bore 1827 of member 1803. Bore 1871 may be generally aligned with bore 1833 of member 1803 and may include a proximal portion 1871-1 of comparatively lesser diameter and a distal portion 1871-2 of comparatively greater diameter. Proximal portion 1871-1 may be comparable in diameter to bore 1833 of member 1803.

Removal device 19 may further comprise a scope guide 1881. Guide 1881, which is also shown separately in FIG. 12, may be a unitary structure, preferably made of a medical-grade stainless steel or a similarly suitable material. Guide 1881 may be a generally tubular member comprising a generally circular side wall 1883 defining a proximal end 1885, a distal end 1887, and a bore 1889, bore 1889 extending all the way from proximal end 1885 to distal end 1887. Proximal end 1885 of guide 1881 may be fixedly mounted within distal portion 1859-2 of scope connector 1851, with the remainder of guide 1881 extending distally through bore 1811 of member 1801, through bore 1827 of member 1803, and through bore 1869 of ring 1861. The length of guide 1881 passing through bore 1811 of first member 1801 may be slidable relative to first member 1801 whereas the lengths of guide 1881 passing through bore 1827 of member 1803 and through bore 1869 of ring 1861 may be fixed relative to member 1803 and ring 1861, respectively.

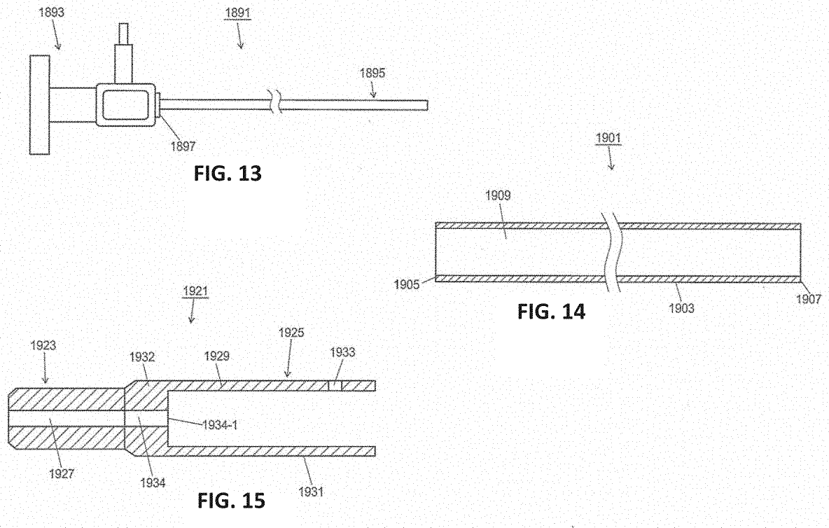

Removal device 19 may further comprise a cystoscope 1891. Cystoscope 1891, which is also shown separately in FIG. 13, may be a wide angle cystoscope. According to one embodiment, cystoscope 1891 may have a field of view of approximately 30-150 degrees, preferably approximately 90-135 degrees, more preferably approximately 105-135 degrees, and more preferably approximately 115 degrees. According to another embodiment, cystoscope 1891 may have a field of view of approximately 180 degrees. Cystoscope 1891 may comprise an eyepiece portion 1893 and a barrel portion 1895. Eyepiece portion 1893 may comprise a distal end 1897 securely mountable within channel 1859 of connector 1851, and barrel portion 1895 may be appropriately dimensioned to extend distally from connector 1851 through member 1801, through member 1803, and through ring 1861 and to terminate proximate to the distal end 1887 of guide 1881. In this manner, cystoscope 1891 may be fixed relative to guide 1881.

Removal device 19 may further comprise a support 1901 (FIGS. 9(a)-9(b)). Support 1901, which is also shown separately in FIG. 14, may be a unitary structure, preferably made of a medical-grade stainless steel or a similarly suitable material. Support 1901 may be a generally tubular member comprising a generally circular side wall 1903 defining a proximal end 1905, a distal end 1907, and a bore 1909, bore 1909 extending all the way from proximal end 1905 to distal end 1907. Proximal end 1905 of support 1901 may be fixedly mounted within distal portion 1871-2 of ring 1861.

Removal device 19 may further comprise a bracket 1921 (FIGS. 9(a)-9(b)). Bracket 1921, which is also shown separately in FIG. 15, may be a unitary structure, preferably made of a medical-grade stainless steel or a similarly suitable material. Bracket 1921 may be shaped to include a proximal portion 1923 and a distal portion 1925. Proximal portion 1923 may be tubular and may be shaped to include a longitudinal channel 1927. Distal end 1907 of support 1901 may be fixedly mounted within channel 1927 of bracket 1921. Distal portion 1925 of bracket 1921 may be bifurcated and may include a top member 1929, a bottom member 1931, and a connecting member 1932, top member 1929 and bottom member 1931 being spaced apart and generally parallel to one another. Top member 1929 may include a transverse opening 1933. Connecting member 1932 may have a bore 1934 aligned with channel 1927.

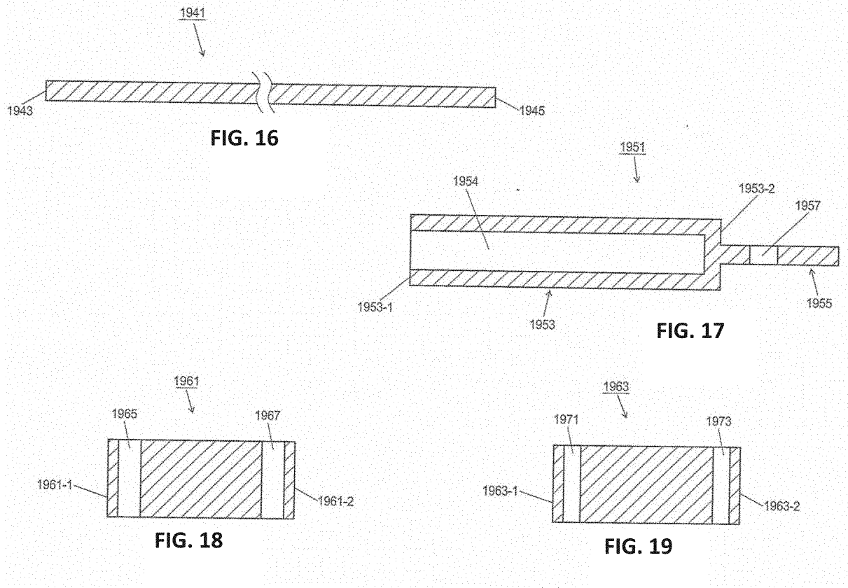

Removal device 19 may further comprise a rod 1941 (FIG. 9(b)). Rod 1941, which is also shown separately in FIG. 16, may be a unitary structure, preferably made of a medical-grade stainless steel or a similarly suitable material. Rod 1941 may be a solid, rigid member shaped to include a proximal end 1943 and a distal end 1945. Rod 1941 may be appropriately dimensioned to be slidably mounted within support 1901, with proximal end 1943 being fixedly mounted within cavity 1817 of member 1801 and with distal end 1945 being adapted to slide back and forth through a distal end 1934-1 of bore 1934.

Removal device 19 may further comprise a connector 1951 (FIG. 9(b)). Connector 1951, which is also shown separately in FIG. 17, may be a unitary structure, preferably made of a medical-grade stainless steel or a similarly suitable material. Connector 1951 may be shaped to include a proximal portion 1953 and a distal portion 1955. Proximal portion 1953 may be tubular and may be shaped to include a channel 1954 extending longitudinally a portion of the way from a proximal end 1953-1 towards a distal end 1953-2. Distal end 1945 of rod 1941 may be fixedly mounted within channel 1954 of connector 1951, and proximal portion 1953 of connector 1951 may be appropriately dimensioned to slide back and forth within bracket 1921. Distal portion 1955 of connector 1951 may be generally flat and elongated and may be disposed in the space between top member 1929 and bottom member 1931 of bracket 1921. Distal portion 1955 of connector 1951 may be shaped to include a transverse opening 1957.

Removal device 19 may further comprise a pair of linking arms 1961 and 1963 (FIGS. 9(b)-9(d)). Arm 1961, which is also shown separately in FIG. 18, may be a unitary structure, preferably made of a medical-grade stainless steel or a similarly suitable material. Arm 1961 may be an elongated flat member shaped to include a first transverse opening 1965 proximate to a proximal end 1961-1 of arm 1961 and a second transverse opening 1967 proximate to a distal end 1961-2 of arm 1961. A pivot pin 1969 may be received within opening 1965 of arm 1961, as well as within opening 1957 of connector 1951, so as to pivotally couple arm 1961 to connector 1951. Arm 1963, which is also shown separately in FIG. 19, may be a unitary structure, preferably made of a medical-grade stainless steel or a similarly suitable material. Arm 1963 may be an elongated flat member shaped to include a first transverse opening 1971 proximate to a proximal end 1963-1 of arm 1963 and a second transverse opening 1973 proximate to a distal end 1963-2 of arm 1963. Pivot pin 1969 may additionally be received within opening 1971 of arm 1963 so as to pivotally couple arm 1963 both to arm 1961 and to connector 1951.

Removal device 19 may further comprise a pair of deflectable jaws 1981 and 1983 (FIGS. 9(a)-9(d)). The jaws can include gripping members such as but not limited to, corresponding teeth 1997, 2017 which can be used to grip or secure an implant. The jaws may also include one or more surface damaging or compromising structures. For example, the surface damaging structure 2003, 2023 can be a needle, knife, sharpened tooth, etc. In some embodiments, the surface damaging structure can be a cannulated needle that can also serve to allow the media within in the implant to escape or otherwise be removed. In some embodiments, having the opening in the needle extend the entire length of the exposed needle structure permits the balloon to continue to deflate even when the needle has penetrated completely through the balloon. Additionally, the orientation of the sharp edge towards the distal end of the grasper has the advantage of preventing lacerating the balloon film during the tensile removal of the deflated or partially deflated balloon thru the sheath. Additionally, the proximity of the needle relative to adjacent teeth can improve the function of the removal system. Specifically, if the space between the tip of the needle and the tip of an adjacent tooth is between 0.05 and 10 times the difference in height between the tip of the needle and the tip of the adjacent tooth. This distance prevents the balloon from “tenting” over the needle and adjacent teeth without needle penetration of the balloon. An example of a larger distance between the tip of the needle and the tip of the adjacent tooth is shown in FIG. 20(e). Needle 2513-3 is taller than adjacent tooth 1997. The distance between the tip of the tooth 1997 and needle 2513-1 or 2513-3 is equal to or greater than the difference in distance between the height of needle 2513-1 or 2513-3 and the height of the adjacent tooth.

Jaw 1981, which is also shown separately in FIGS. 20(a) through 20(d), may comprise an elongated member 1985 (which may be, for example, approximately 1.55-2.5 inches in length), preferably made of a medical-grade stainless steel or a similarly suitable material. Member 1985 may be shaped to include a proximal portion 1987 and a distal portion 1989. Proximal portion 1987, which may comprise a generally flat and arcuate arm, may be shaped to include a first transverse opening 1991 proximate to a proximal end 1987-1 of proximal portion 1987 and a second transverse opening 1993 spaced distally a short distance from first transverse opening 1991. A pivot pin 1995 may be received within opening 1991 of proximal portion 1987, as well as within opening 1967 of arm 1961, so as to pivotally couple jaw 1981 to arm 1961. A pivot pin 1996 may be received within opening 1993 of proximal portion 1987, as well as within opening 1933 of bracket 1921, so as to pivotally couple jaw 1981 to bracket 1921. Distal portion 1989 of member 1985 may be shaped to include a row of teeth 1997 facing towards jaw 1983, the row of teeth 1997 extending proximally from approximately the distal end of distal portion 1989. Each tooth 1997 may extend substantially across the width of distal portion 1989 and may have a height of, for example, approximately 1-10 mm, preferably approximately 5 mm. Each tooth 1997 may have a dulled peak 1997-1 that has a radius of, for example, 0.001-0.250 inch, preferably 0.005-0.050 inch, more preferably 0.010-0.25 inch. A first transverse opening 1999 may be provided in distal portion 1989 amongst teeth 1997, and a second transverse opening 2001 may be provided in distal portion 1989 amongst teeth 1997, first and second transverse openings 1999 and 2001 being spaced apart from one another by a short distance. A cannulated needle 2003 may be fixedly mounted in transverse opening 1999, needle 2003 having a sharpened end 2003-1 facing towards jaw 1983. Preferably, needle 2003 has a height that exceeds the height of teeth 1997 so that sharpened end 2003-1 extends beyond dulled peaks 1997-1. Needle 2003 may have an inner diameter of, for example, approximately 0.0005-0.500 inch, preferably approximately 0.005-0.250 inch, more preferably approximately 0.010-0.050 inch, and may have an outer diameter of, for example, approximately 0.001-0.750 inch, preferably approximately 0.010-0.300 inch, more preferably approximately 0.015-0.075 inch.

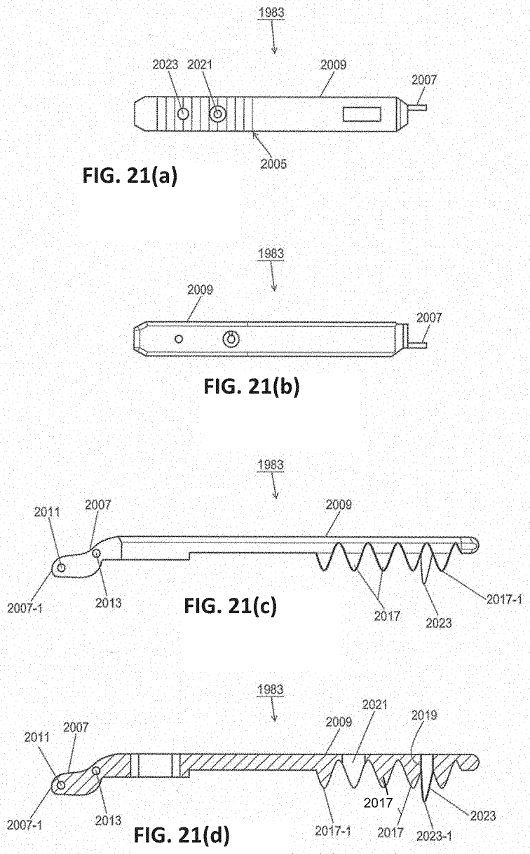

Jaw 1983, which is also shown separately in FIGS. 21(a) through 21(d), may comprise an elongated member 2005 (which may be, for example, approximately 1.55-2.5 inches in length), preferably made of a medical-grade stainless steel or a similarly suitable material. Member 2005 may be shaped to include a proximal portion 2007 and a distal portion 2009. Proximal portion 2007, which may comprise a generally flat and arcuate arm, may be shaped to include a first transverse opening 2011 proximate to a proximal end 2007-1 of proximal portion 2007 and a second transverse opening 2013 spaced distally a short distance from first transverse opening 2011. A pivot pin 2015 may be received within opening 2011 of proximal portion 2007, as well as within opening 1973 of arm 1963, so as to pivotally couple jaw 1983 to arm 1963. Pivot pin 1996 may be received within opening 2013 of proximal portion 2007, as well as within opening 1933 of bracket 1921, so as to pivotally couple jaw 1983 to bracket 1921. In this manner, proximal movement of rod 1941, which may be caused by pivotal movement of ring portion 1807 of member 1801 towards ring portion 1823 of member 1803, may cause arms 1961 and 1963 to pivot towards each other which, in turn, may cause jaws 1981 and 1983 to pivot towards each other. On the other hand, distal movement of rod 1941, which may be caused by pivotal movement of ring portion 1807 of member 1801 away from ring portion 1823 of member 1803, may cause arms 1961 and 1963 to pivot away from one another which, in turn, may cause jaws 1981 and 1983 to pivot away from one another. Jaws 1981 and 1983 may open to an angle of, for example, approximately 20-150 degrees.

Distal portion 2009 of member 2005 may be shaped to include a row of teeth 2017 facing towards jaw 1981. The row of teeth 2017 may be staggered relative to teeth 1997 so that the peaks 1997-1 of teeth 1997 may be aligned with the spaces between teeth 2017 when jaws 1981 and 1983 are closed and so that the peaks 2017-1 of teeth 2017 may be aligned with the spaces between teeth 1997 when jaws 1981 and 1983 are closed. Each tooth 2017 may extend substantially across the width of distal portion 2009 and may be shaped and dimensioned similarly to each of teeth 1997. A first transverse opening 2019 may be provided in distal portion 2009 amongst teeth 2017, and a second transverse opening 2021 may be provided in distal portion 2009 amongst teeth 2017. Opening 2019 may be appropriately positioned and appropriately dimensioned to receive cannulated needle 2003 of jaw 1981 when jaws 1981 and 1983 are closed. (By receiving the sharpened end 2003-1 of needle 2003, opening 2019 facilitates and promotes full closure of jaws 1981 and 1983 around an inflated device 17, as opposed to having needle 2003 be deflected from the compressed and inflated device 17.) Opening 2019 may have an inner diameter of, for example, approximately 0.002-0.100 inch, preferably 0.010-0.300 inch, more preferably 0.015-0.100 inch. Opening 2021 may be aligned with opening 2001 of jaw 1981 when jaws 1981 and 1983 are closed, and a cannulated needle 2023 may be fixedly mounted in opening 2021 so as to be receivable within opening 2001 of jaw 1981 when jaws 1981 and 1983 are closed. Cannulated needle 2023 may have a sharpened end 2023-1 facing towards jaw 1981, and needle 2023 and opening 2001 may be dimensioned similarly to needle 2003 and opening 2019, respectively.

Preferably, teeth 1997 and 2017 are dimensioned appropriately so that, when jaws 1981 and 1983 are closed, a small gap 2018 (seen best in FIG. 9(d)) is left between the respective rows of teeth 1997 and 2017 that enables device 17 to be trapped between teeth 1997 and 2017 while minimizing any tearing of device 17 by teeth 1997 and 2017. In this manner, device 17 may be securely held or gripped between teeth 1997 and 2017 while cannulated needles 2003 and 2023 puncture device 17. Moreover, because needles 2003 and 2023 are cannulated, the fluid contents of device 17 may be quickly evacuated from device 17 through needles 2003 and 2023 without having needles 2003 and 2023 plug the same puncture holes they create.

It is to be understood that, although cannulated needles 2003 and 2023 are described herein as being used to puncture device 17, other puncturing devices, such as, but not limited to, blades, scissors, pins, hooks, or the like, may alternatively or additionally be used.

In addition, it is to be understood that, although cannulated needles 2003 and 2023 are described herein as being oriented generally perpendicular to members 1985 and 2005, respectively, cannulated needles 2003 and 2023 need not be so oriented and may be oriented, for example, so that sharpened ends 2003-1 and 2023-1 are angled towards proximal portions 1987 and 2007, respectively.

Additionally, it is to be understood that, although both jaw 1981 and jaw 1983 are described herein as being movable, one could make one of jaws 1981 and 1983 stationary and the other of jaws 1981 and 1983 movable.

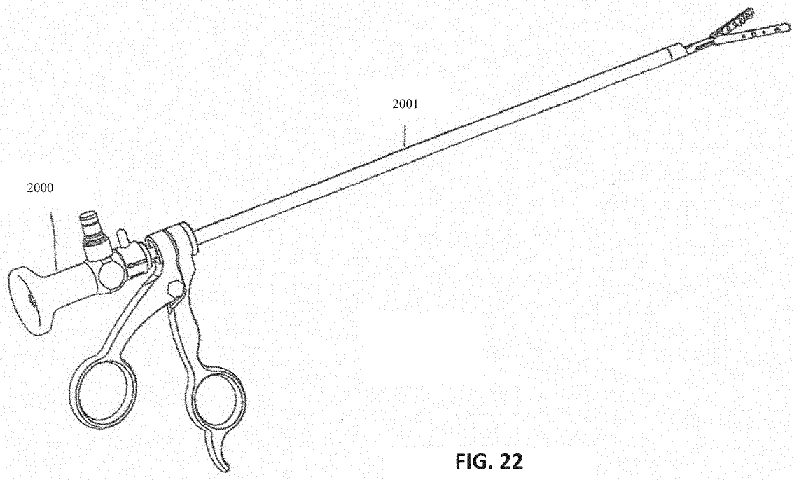

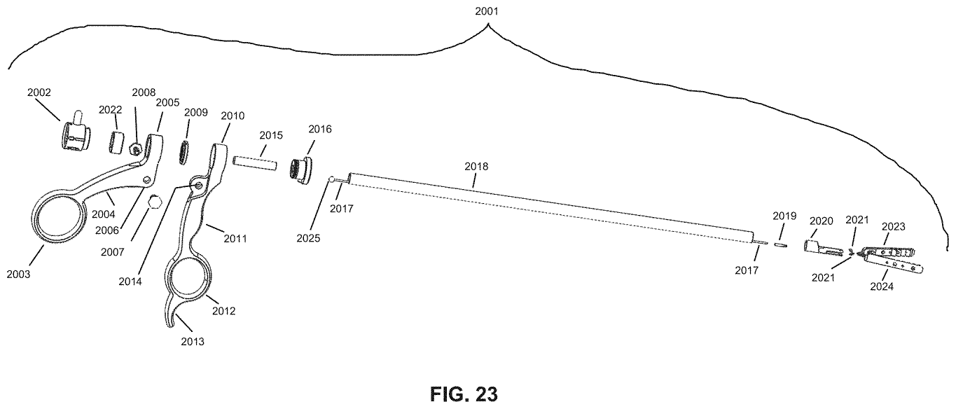

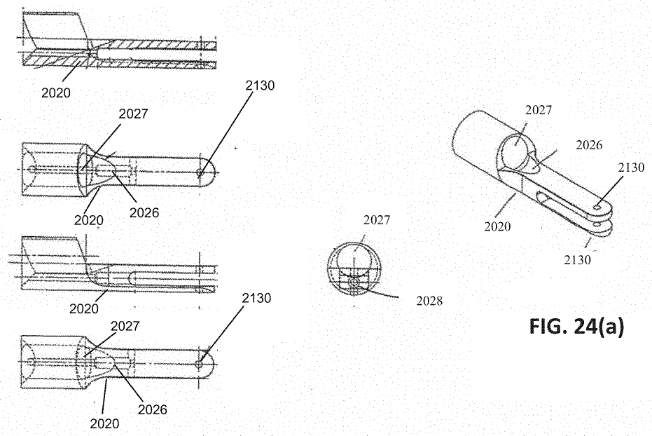

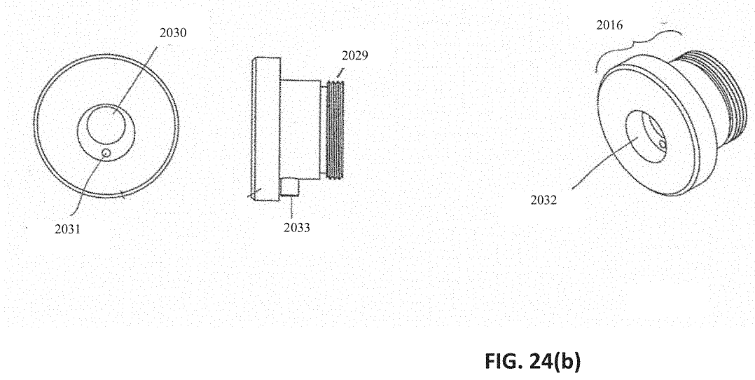

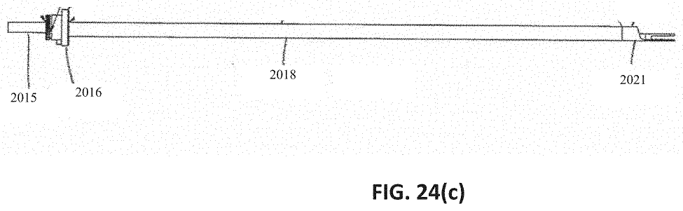

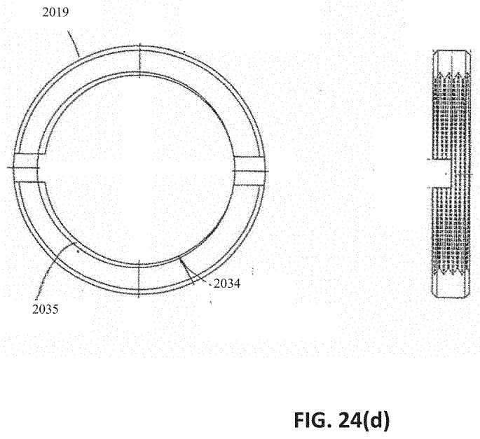

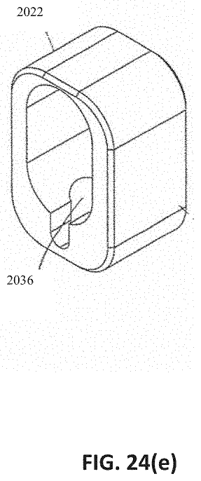

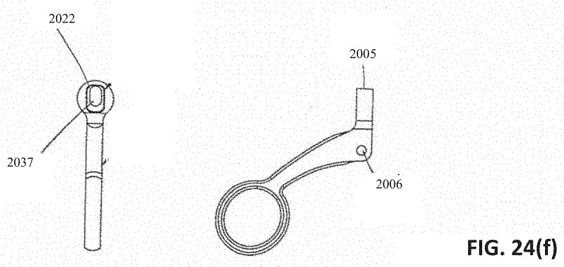

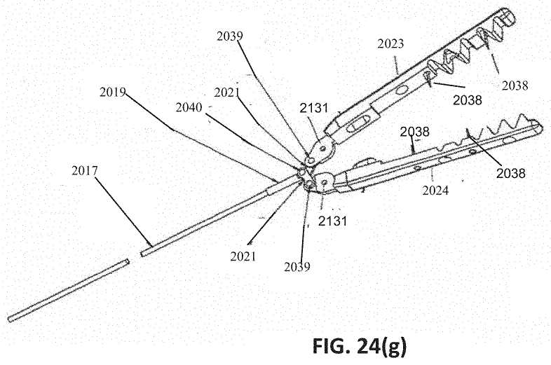

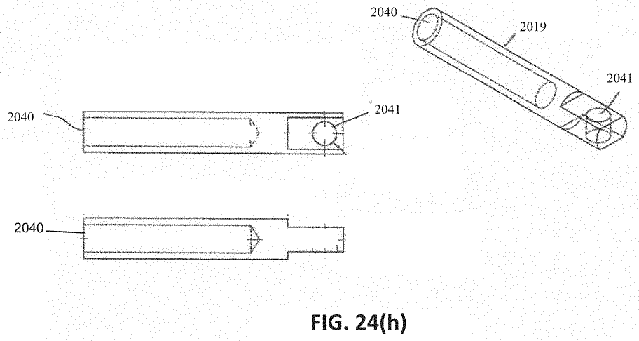

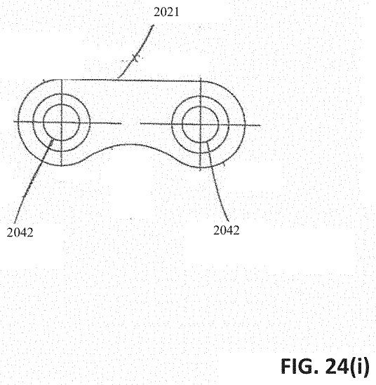

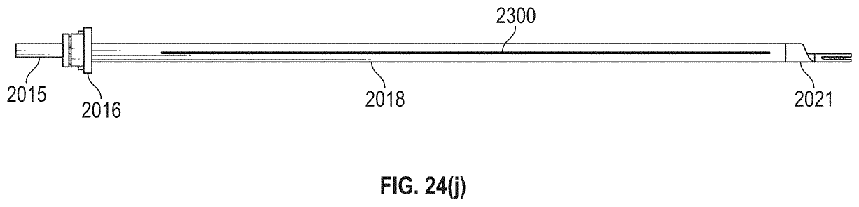

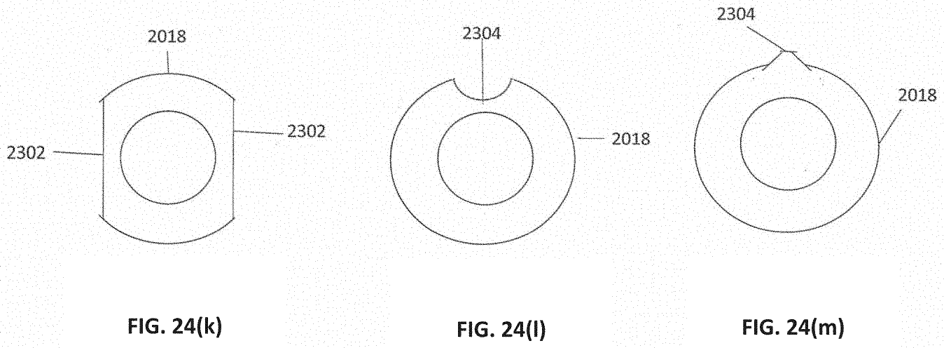

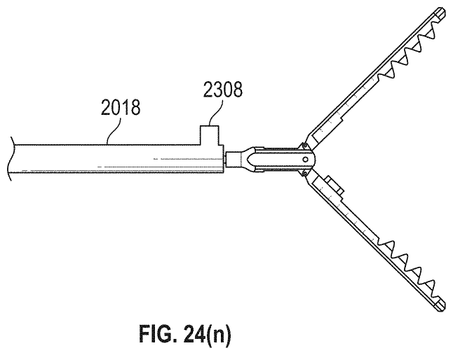

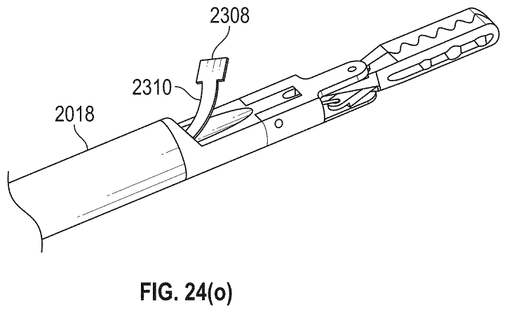

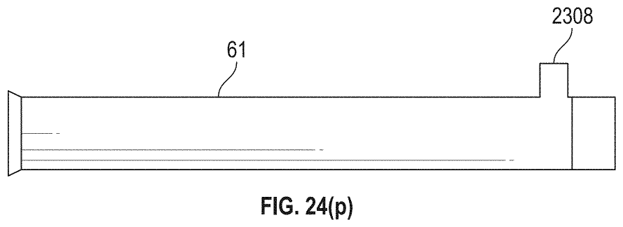

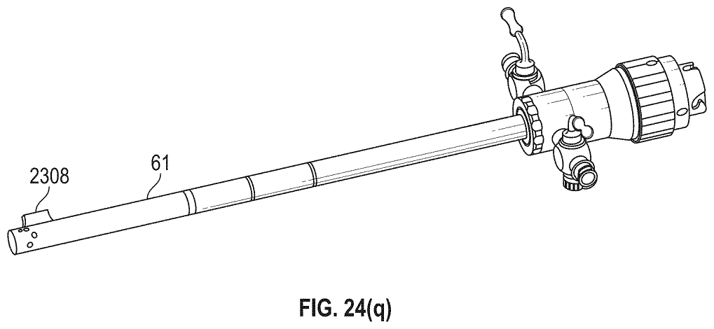

FIG. 22 illustrates an embodiment of a removal system 2001 and a scope 2000. FIG. 23 illustrate an exploded view of the removal device 2001 shown in FIG. 22. FIG. 24(a)-24(q) illustrate various individual components, shown in exploded view in FIG. 23. One or more components of the removal device 2001 can be similar to the removal system shown in FIGS. 9(a)-9(b). As such, the description of one or more components of the removal device 19 of FIG. 9(a)-9(b) can be applicable to the corresponding one or more components of the removal device 2001 of FIGS. 22, 23, and 24(a)-24(q). For example, the scope 2000 of FIG. 22 can include an eyepiece portion that can, in certain embodiments, be similar to the eyepiece portion 1893 of FIG. 9(b). Furthermore, in certain embodiments, other components of the removal device 2001 of FIGS. 22, 23, and 24(a)-24(q) can correspond to components of the removal device 19 of FIG. 9(a)-9(b): for example, the scope adapter 2002 of FIG. 23 can correspond to the connector 1851 of FIG. 9(a); the moving handle 2004 of FIG. 23 can correspond to the first member 1801 of FIG. 9(b); the pin 2007 of FIG. 23 can correspond to the pin 1840 of FIG. 9(b); the handle 2011 of FIG. 23 can correspond to the second member 1803 of FIG. 9(b); the proximal hub 2016 of FIG. 23 can correspond to the ring 1861 of FIG. 9(b); the outer tube 2018 of FIG. 23 can correspond to the scope guide 1881 of FIG. 9(b); the pull wire 2017 of FIG. 23 can correspond to the rod 1941 of FIG. 9(b); the distal tip 2020 of FIG. 23 can correspond to the bracket 1921 of FIG. 9(b); the connector 2019 of FIG. 23 can correspond to the connector 1951 of FIG. 9(b); the link or links 2021 of FIG. 23 can correspond to the pair of linking arms 1961 and 1963 of FIG. 9(b); and the jaws 2023 and 2024 of FIG. 23 can correspond to the pair of jaws 1981 and 1983 of FIG. 9(b). Some of the components of the removal device 2001 shown in FIG. 23 can be the same as or identical to components of the removal device 19 shown in FIG. 9(b). Some of the components of the removal device 2001 shown in FIG. 23 can be similar or substantially similar, e.g., in structure, function, finish, material, etc., to components of the removal device 19 shown in FIG. 9(b). While some of the components of the removal device 2001 shown in FIG. 23 can be similar or the same as components of the removal device 19 shown in FIG. 9(b), none are required to be similar, substantially similar, or identical.

The various components of the removal device 2001 shown in FIG. 23 will now be described in a generally proximal to distal direction (e.g., in a direction from the handle to the jaws).

The scope adapter 2002 of the removal device 2001 can be configured to accept and connect the scope 2000 to the removal device 2001. The scope adapter 2002 can be attached to an end, e.g., the proximal end, of the proximal tube 2015, thereby providing a guide for the scope as it is placed within the removal device 2001. The scope adapter 2002 can be attached to the proximal tube 2015 by welding. In some embodiments, the scope adapter 2002 is attached to the proximal tube 2015 using a threaded attachment, clips, hooks, friction fit, epoxies, glues or cements. In some embodiments, the scope adapter 2002 is formed as a unitary structure, e.g., monolithically, with the proximal tube 2015.

The distal end of the proximal tube 2015, e.g., the end of the proximal tube 2015 not attached to the scope adapter 2002, can be attached to the proximal end of the proximal hub 2016. Various views of the proximal hub are shown in FIG. 24(b). The proximal hub 2016 can be attached to the proximal tube 2015 by welding. In some embodiments, the proximal hub 2016 is attached to the proximal tube 2015 using a threaded attachment, clips, hooks, friction fit, epoxies, glues or cements. In some embodiments, the proximal hub 2016 is formed as a unitary structure, e.g., monolithically, with the proximal tube 2015. As shown in FIG. 24(b), the proximal hub has an opening 2032 on its distal end that is configured or sized to accept or fit the outer tube 2018 (shown in FIG. 24(c)). The distal surface of the proximal hub 2016 can form a substantially flat surface (in which the opening 2032 is inset). Opposite its distal end, e.g., moving proximally, the proximal hub has a proximal shoulder defined, at least partially, by a decrease in outer diameter from a first outer diameter to a second outer diameter. The proximal hub 2016 can also include a key 2033 configured to maintain alignment of one or more pieces of the removal device 2001 with respect to one or more other pieces of the removal device 2001. For example, the key 2033 can be configured to mate with a corresponding recess in the handle 2011, thereby ensuring alignment of the handle 2011 with respect to the key (e.g., and with respect to the outer tube 2018). The key 2033 can directly abut the proximal shoulder of the proximal hub 2016. The key 2033 can be offset, proximally, from the proximal shoulder of the proximal hub 2016, e.g., such that some space exists between the key 2033 and the proximal shoulder of the proximal hub. The key 2033 of the proximal hub 2016 can advantageously serve to maintain alignment of the handle 2011 thereby permitting or ensuring alignment of the jaws 2023 and 2024. The proximal end of the proximal hub comprises a set of threads 2029 and at least one opening. As shown in FIG. 24(B), the threads 2029 on the proximal end of the proximal hub can be on the outer surface of the proximal hub 2016, e.g., a portion of the outer surface of the proximal hub 2016 can be threaded in a proximal to distal direction). Of course, any other type of connector can be used such as, but not limited to clips, hooks, friction fit, epoxies, glues or cements. The at least one opening in the proximal end of the proximal hub 2016 can advantageously be aligned with, or contained within, the opening 2032 on the distal end of the proximal hub 2016. In this way, the at least one opening can communicate with the interior of the outer tube 2018 when the outer tube 2018 is attached to or coupled with the proximal hub 2016. In some embodiments, the proximal end of the proximal hub 2016 comprises a proximal tube opening 2030, configured to accept or fit the proximal tube 2015 (this is where, in some embodiments, the proximal tube 2015 can be or is attached to the proximal hub 2016, as discussed herein). In some embodiments, the proximal tube opening 2030 extends, e.g., axially or longitudinally extends, through the entirety of the proximal hub 2016. In some embodiments, the proximal end of the proximal hub 2016 comprises a wire opening 2031, configured to accept, fit, or pass the pull wire 2017, such that the pull wire 2017 can freely move along or through the wire opening 2031. In some embodiments, the pull wire 2017 extends, e.g., axially or longitudinally extends, through the entirety of the proximal hub 2016. As disclosed herein, the at least one opening, e.g., which can comprise one or more of the proximal tube opening 2030 and the wire opening 2031, can be aligned with the opening 2032. Alignment of the one or more opening with the opening 2032 can advantageously permit the scope 2000 and the pull wire 2017 to travel within the inner diameter of the outer tube 2018 and the pull wire 2017 to travel outside the proximal tube 2015, when assembled.

FIG. 24(c) illustrates the proximal hub 2016 accepting the proximal end of the outer tube 2018 (e.g., on the distal end of the proximal hub 2016 in the opening 2032) and accepting the distal end of the proximal tube 2015 (e.g., on the proximal end of the proximal hub 2016 in the proximal tube opening 2030). Thus, as shown in FIG. 24(c), the inner passage of the proximal tube 2015 is aligned with the proximal tube opening 2030, which is aligned with the opening 2032, thereby advantageously creating a patent opening from the proximal end of the proximal tube 2015 to the distal end of the outer tube 2018, such that the scope 2000 can be advanced therethrough.

With further reference to FIG. 24(c), a distal tip 2021 is attached to the distal end of the outer tube 2018. In some embodiments, the distal tip 2020 is welded to the distal end of the outer tube 2018. In some embodiments, the distal tip 2020 is attached to the distal end of the outer tube 2018 using a threaded attachment, clips, hooks, friction fit, epoxies, glues or cements. In some embodiments, the distal tip 2020 is formed as a unitary structure, e.g., monolithically, with the distal end of the outer tube 2018. FIG. 24(a) shows a number of different views of the distal tip 2020.

With reference to FIG. 24(a), the proximal end of the distal tip 2020, e.g., the end that is configured to couple to the outer tube 2018, can comprise at least one opening. As shown in FIG. 24(a), in some embodiments, the proximal end of the distal tip 2020 comprises a scope opening 2027 configured to receive or accept an optical scope, e.g., the scope 2000 passed through the outer tube 2018. The scope opening 2027 can extend all the way through the distal tip 2020, such that a passage is defined or remains open from the proximal end of the distal tip 2020 to the distal end of the distal tip 2020. In some embodiments, the proximal end of the distal tip 2020 comprises a pass through hole 2028 configured to accept, or receive, or permit passage of the pull wire 2017 therethrough. The distal end of the distal tip 2020 comprises a jaw-holding structure. As shown in FIG. 24(a), the distal end of the distal tip 2020 comprises a pair of wings 2301 having aligned holes 2130. The pair of wings 2301 can be configured to accept a pair of jaws therebetween, e.g., jaw 2023 and jaw 2024 (shown in FIG. 24(g)). The jaws can be held between the pair of wings 2301 by a pin (not shown) that extends through the aligned holes 2130 in the wings 2301. Further details regarding attachment of the jaws to the distal tip 2020 are discussed elsewhere herein.

FIG. 23 illustrates the removal device 2001 assembled, such that the jaws, e.g., jaw 2023 and jaw 2024 are attached to and held by the distal tip 2020. As can be seen, the scope opening 2027 of the distal tip 2020 can be blocked, e.g., partially blocked or obscured by one or more of the distal elements of the removal device 2001, e.g., the jaws. Therefore, one or more distal elements of the removal device 2001 can obstruct, e.g., partially obstruct, the view from the lens of the scope 2000 when the scope 2000 is being used, e.g., when the scope 2000 is inserted through the device, through the outer tube 2018 and to the distal tip 2020. Turning again to FIG. 24(a), to minimize or ameliorate the impact of field of view obstruction, the distal tip 2020 can include a scoop 2026 also referred to herein in as recess 2026. The recess or scoop 2026 can be a continuous curved surface that is opposite the distal end of the scope opening 2027. The scoop 2026 can advantageously minimize direct reflection of light from the scope, thereby improving imaging capabilities. In some embodiments, the scoop 2026 is a physically hollowed out area that physically enlarges the field of view of the scope inserted through the scope opening 2027 (e.g., stated alternatively, the scoop 2026 defines an absence of blocking material). In some embodiments, the scoop 2026 can incorporate a coating or finish that reduces reflection. For example, the scoop 2026 can have a surface colored so as to reduce light reflection, e.g., the scoop 2026 may have a black surface. The scoop 2026 may have a roughened surface that reduces light reflection or diffuses light reflection. In some embodiments, the surface of the scoop 2026 is configured such that the distal end, e.g., lens, of a scope inserted through the scope opening 2027 rests at least 1 mm from the surface of the scoop 2026. In some embodiments, the surface of the scoop 2026 is configured such that the distal end, e.g., lens, of a scope inserted through the scope opening 2027 rests at least 2 mm from the surface of the scoop 2026. In some embodiments, the surface of the scoop 2026 is configured such that the distal end, e.g., lens, of a scope inserted through the scope opening 2027 rests a distance from the surface of the scoop 2026, the distance being between about 0.5-4 mm, between about 0.75-3.5 mm, between about 1-3 mm, between about 1.25-2.5 mm, or between about 1.5-2 mm. Increasing the distance between the lens of the scope and the surface of the scoop 2026 can advantageously reduce visual obstruction in front of the lens. The scoop or recess 2026 can have a surface configured to absorb light.

As discussed herein, the distal tip 2020 can include wings having holes extending therethrough. As shown in FIG. 24(a), the distal tip 2020 can have two horizontally oriented wings 2301 having holes 2130 extending therethrough. The holes 2130 in the wings 2301 of the distal tip 2020 can be aligned such that a pin (not shown) can be placed therebetween. While the wings shown in FIG. 24(a) are horizontally oriented (e.g., at an angle of zero with respect to the horizon), the wings can be vertically oriented (e.g., at an angle of 90 with respect to the horizon). In some embodiments, the wings have an oblique orientation, e.g., have an angle with respect to the horizon of between 0-90 (e.g., greater than 0 but less than 90) or between 90-180 (e.g., greater than 90 but less than 180). The wings and the holes 2130 therein can be used to secure a jaw assembly, e.g., jaw assembly shown in FIG. 24(g), to the distal tip 2020.

FIG. 24(g) illustrates a jaw assembly that can be used with the various removal devices disclosed herein. The jaw assembly includes a pull wire 2017, a pull wire linkage 2019, a link or links 2021, and a pair of jaws, e.g., jaw 2023 and jaw 2024. FIG. 24(h) illustrates the pull wire linkage 2019. FIG. 24(i) illustrates a link or links 2021.

As discussed herein, one or more of jaw 2023 and jaw 2024 comprises at least one needle 2038. In some embodiments, at least one of jaw 2023 and jaw 2024 comprises two open needles 2038 that pass through the width of the jaw, e.g., laterally through the width of the jaw.

As will be easily appreciated with reference to FIG. 24(g), the pull wire 2017 can be coupled to the pull wire linkage 2019. While the pull wire 2017 and the pull wire linkage 2019 are shown as separate components, in some embodiments, the pull wire 2017 and the pull wire linkage 2019 are formed as a single component, e.g., the pull wire 2017 can terminate in a tongue with a hole therein. In some embodiments, the pull wire linkage 2019 is welded to the distal end of the pull wire 2017. In some embodiments, the pull wire linkage 2019 is attached to the distal end of the pull wire 2017 using a threaded attachment, clips, hooks, friction fit, epoxies, glues or cements. In some embodiments, the pull wire linkage 2019 is formed as a unitary structure, e.g., monolithically, with the distal end of the pull wire 2017. As shown in FIG. 24(h), the pull wire linkage 2019 includes a cavity 2040 configured to accept the pull wire 2017 and a tongue with a hole 2041 therethrough. The hole 2041 of the tongue is configured to accept a pin 2039. The pin 2039, which can pass through and/or be help by the hole 2041 in the pull wire linkage 2019 can also be used to connect the pull wire linkage 2019 to one or more link or links 2021. FIG. 24(i) illustrates an embodiment of a link or links 2021 that can be used in conjunction with the removal device 2001. The link or links 2021 includes two linkage holes 2042, including a proximal linkage hole 2042 and a distal linkage hole 2042. The proximal linkage hole 2042 of each link or links 2021 can be connected to the hole 2041 of the pull wire linkage 2019 by using a pin 2039. As shown in FIG. 24(g), the distal linkage hole 2042 of each link or links 2021 can be connected to a proximal end of each of the jaw 2023 and jaw 2024, e.g., a hole in the proximal end of the jaws, using a pin 2039. In this way, when the pull wire linkage 2019 is advanced distally, e.g., by advancing the pull wire 2017, the jaw 2023 and jaw 2024 close or come together. Stated alternatively, when the pull wire 2017 is in its distal most position the jaws, e.g., jaw 2023 and jaw 2024, are in a position closest to each other. By contrast, when the pull wire linkage 2019 is retracted or pulled proximally, e.g., by pulling the pull wire 2017, the jaw 2023 and jaw 2024 open or extend apart. Stated alternatively, when the pull wire 2017 is in its proximal most position, the jaws, e.g., jaw 2023 and jaw 2024, are in a position furthest apart from each other.

FIG. 24(c) illustrates an outer tube 2018 with a distal tip 2020 attached to its distal end and a proximal hub 2016 attached to its proximal end. Attached to the proximal side of the proximal hub 2016 is the proximal tube 2015. With reference to FIG. 202, a handle 2011 can be fixed to the assembly shown in FIG. 24(c), e.g., to the threaded side of the proximal hub 2016. In some embodiments, the handle 2011 can simply be glued or welded in place. In some embodiments, a handle ring 2010, e.g., a cylindrical portion with a bore, of the handle 2011 slips over the threads 2029 of the proximal hub 2016 and butts up against the proximal shoulder of the proximal hub 2016. The handle ring 2010 of the handle 2011 allows the proximal tube 2015 and the pull wire 2017 (and, possibly, a portion of the 2016) to travel or extend proximally past the proximal most surface of the handle ring 2010 of the handle 2011 (e.g., through the handle ring 2010). Once the handle ring 2010 is in place, a nut 2009 (an example of which is shown in FIG. 24(d)) can be threaded onto the threads 2029 of the proximal hub 2016 to fixedly secure the handle ring 2010 of the handle 2011 in place (and therefore the handle 2011 with respect to various other portions of the device, e.g., the handle ring 2010, the proximal hub 2016, etc.). While it can take many different functional forms, the handle 2011 can include, in addition to the handle ring 2010, a ring portion 2012 a finger rest 2013, and a handle hole 2014.

With continued reference to FIG. 23, a moving handle 2004 can be rotatably attached to the handle 2011. FIG. 24(f) shows various views of the moving handle 2004, separated from the rest of the removal device 2001. While it can take many different functional forms, the moving handle 2004 can include, among other things, a moving handle ring 2005, a ring portion 2003, a moving handle hole 2006, and a recess configured to hold a retaining block 2022.

As shown in FIG. 24(e), the retaining block 2022 includes a bore or hole that can be generally aligned with the longitudinal axis of the outer tube 2018. The bore or hole in the retaining block 2022 can be configured to permit the proximal tube 2015 to pass through one or more of the retaining block 2022 and the scoop 2026 (which holds the retaining block 2022). A receptacle 2036 can be disposed on an inner surface of the retaining block 2022. The receptacle 2036 can, in turn, be shaped, seized, or generally configured to accept and/or hold the pull wire stop 2025, which, as shown in FIG. 23, can be an portion of the pull wire 2017 on the proximal end of the pull wire 2017 having an increased diameter (e.g., a ball, cylinder, or other portion fixed to or integrally formed with the pull wire 2017).

In some embodiments, the retaining block 2022 and the moving handle 2004 are separate components, e.g., as discussed, the moving handle 2004 can have a recess shaped, sized, or generally configured to hold or accept the retaining block 2022. In some embodiments, the features of the retaining block 2022 can be integral to the moving handle 2004. For example, the moving handle 2004 can have a moving handle ring 2005 having a receptacle 2036 on its inner surface.

In some embodiments, the moving handle 2004 is rotatably attached to handle 2011 by a pin 2007 passing through the handle hole 2014 of the handle 2011 and the moving handle hole 2006 of the moving handle 2004.

As will be understood with respect to the foregoing discussion and with reference to FIG. 23, due to the hinged or rotating attachment of the moving handle 2004 and the handle 2011 (which is fixedly attached to the proximal hub 2016 and 108, movement moving handle 2004 with respect to the handle 2011 will cause the jaw 2023 and jaw 2024 to move open and closed. For example, when the ring portion 2003 of the moving handle 2004 is moved closer to the ring portion 2012 of the handle 2011, the moving handle ring 2005 of the moving handle 2004 is moved apart from the handle ring 2010 of the handle 2011. Further, when the ring portion 2003 of the moving handle 2004 is moved away from the ring portion 2012 of the handle 2011, the moving handle ring 2005 of the moving handle 2004 is moved towards the handle ring 2010 of the handle 2011. Because the receptacle 2036 (e.g., in the retaining block 2022 or the moving handle ring 2005, depending on construction) holds the pull wire stop 2025 of the pull wire 2017, movement of the moving handle ring 2005 in a proximal and/or distal direction causes corresponding movement of the pull wire 2017 in a corresponding proximal and/or distal direction. Thus, bringing the ring portion 2003 of the moving handle 2004 close to the ring portion 2012 causes the moving handle ring 2005 (and the scoop 2026

FIGS. 24(j), 24(k),24(l) show axial views of embodiments where the outer tube 2018 has a drain/fill feature 2300 that allows liquid to flow between the outer tube 2018 and the sheath. In some embodiments, the drain/fill feature 2300 allows liquid to drain and/or fill the bladder. The drain/fill feature 2300 can be a flattened surface on the outer tube 2018, an impression on the outer tube 2018, a ridge, or any other feature that creates space and/or a channel between the outer tube 2018 and sheath. FIG. 24(k) illustrates an embodiment where a flattened surface 2302 on the outer tube 2018 acts as a drain/fill feature 2300. FIG. 24(k) illustrates an embodiment where an impression 2304 on the outer tube 2018 acts as a drain/fill feature 2300. FIG. 24(m) illustrates an embodiment where a ridge 2306 acts as a drain/fill feature 2300. The drain/fill feature 2300 can be in fluid communication with the sheath's drain port and fluid extension line 281 as shown in FIG. 8(a). In certain embodiments the drain/fill feature can extend the length of the outer tube or in certain embodiments a partial length of the outer tube.

In lieu and/or in addition to an optical scope, FIG. 24(n) and FIG. 24(o) illustrate how cameras can be used with the embodiments described herein. FIG. 24(n) illustrates an embodiment where a camera 2308 can also be placed on or integrated within the outer tube 2018 of removal device itself as schematically illustrated in FIG. 24(n). FIG. 24(o) illustrates an example where a camera wire 2310 with a camera 2308 can be fed though the passageway of the outer tube 2018. FIG. 24(p) and FIG. 24(q) show embodiments that use a camera 2308 attached to the elongate sheath 61 of the removal device.

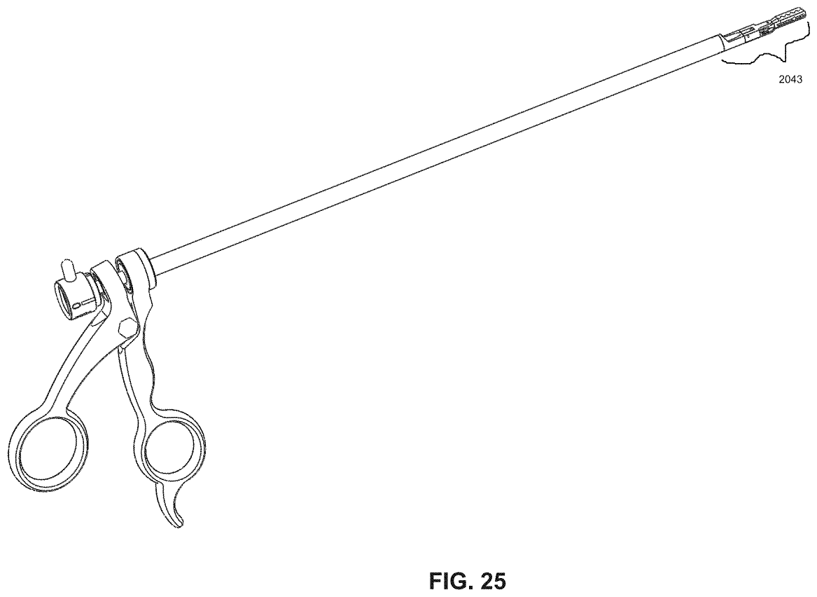

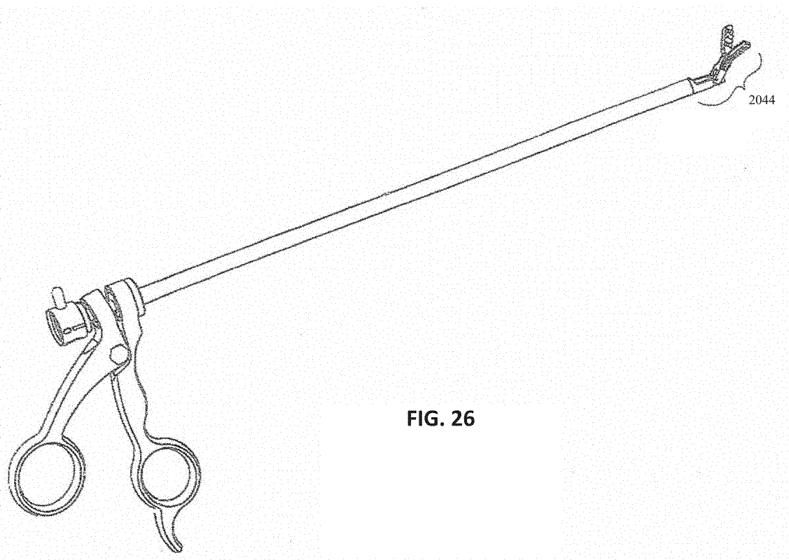

FIGS. 25 and 26 illustrate another embodiment of a removal device. The removal device shown in FIGS. 25 and 26 has jaws 2043, 2044 that can both open laterally and deflect vertically (or, as discussed herein, the jaws can open at any angle with respect to the horizon, e.g., when the handle is vertical, and the deflection can be in any angle). FIG. 25 illustrates the removal device with the jaws closed and aligned along the longitudinal axis of the device 2043, e.g., having zero degrees of vertical deflection. FIG. 26 illustrates the removal device with the jaws open and deflected vertically 2044, e.g., having a non-zero vertical deflection. As described in FIGS. 9(a), 9(b), 45, 46, and 47, in some embodiments, the jaws 2043 can have gripping members such as teeth and/or surface damaging structures, such as, hollow teeth, hollow needles, or cannulated needles, and puncturing members.

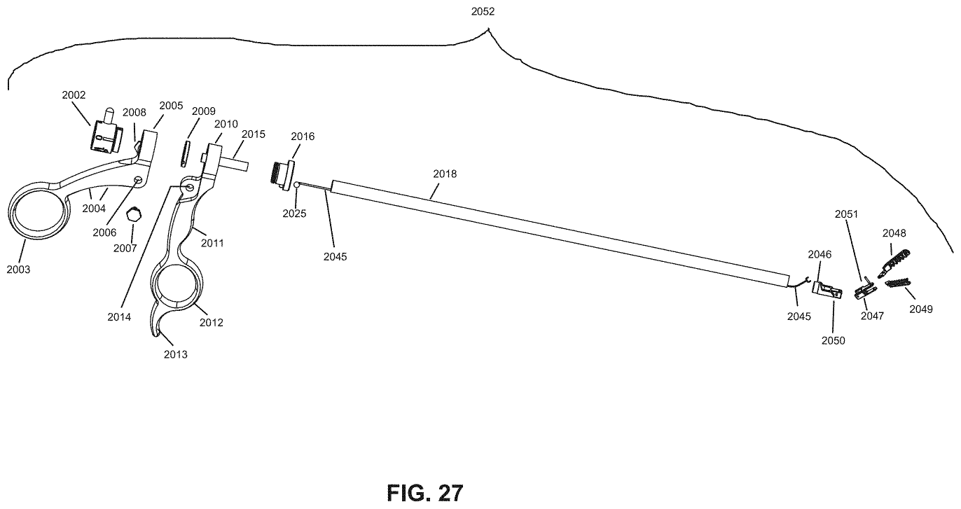

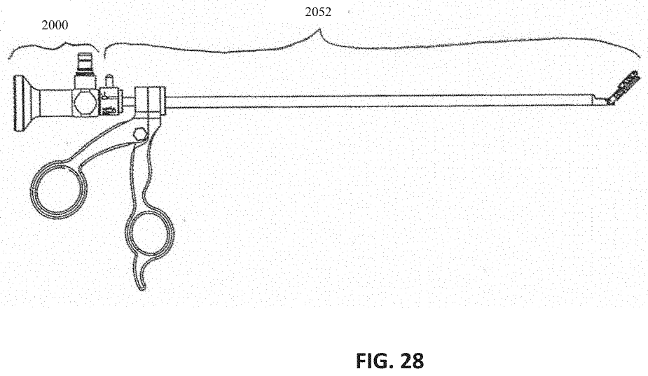

FIG. 27 illustrates an exploded view of the removal device 2052 shown in FIGS. 25 and 26. One or more components of the removal device 2001 can be similar to the removal system shown in FIG. 23. For example, the removal device 2052 can have: a scope adapter 2002; a retaining block 2022; a moving handle 2004 having a moving handle ring 2005, a moving handle hole 2006, and a ring portion 2003; a handle 2011 having a handle ring 2010, a handle hole 2014, a ring portion 2012 and a finger rest 2013; a pin 2007 configured to pass through the moving handle hole 2006 and the handle hole 2014 to connect the moving handle 2004 to the handle 2011; proximal tube 2015; a proximal hub 2016; an outer tube 2018; and a pull wire stop 2025. Some of the components of the removal device 2052 shown in FIG. 27 can be similar or substantially similar, e.g., in structure, function, finish, material, etc., to components of the removal device 2001 shown in FIG. 23. While some of the components of the removal device 2052 shown in FIG. 27 can be similar or the same as components of the removal device 2001 shown in FIG. 23, none are required to be similar, substantially similar, or identical. As shown in FIG. 28, the removal device 2052 of FIGS. 25-27 can be used in conjunction with a scope 2000 as discussed with respect to the various removal devices disclosed herein. The removal device 2052 may incorporate a 2326, which may be configured as described above.

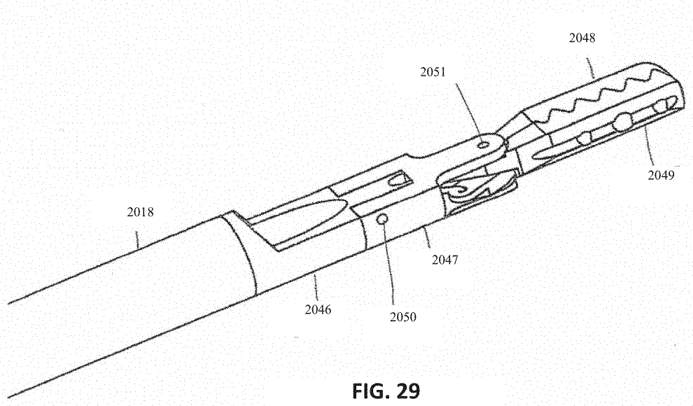

In addition to the components mentioned, the removal device 2052 can include a distal portion, similar to the distal tip 2020 discussed in connection with the removal device 2001 of FIG. 23. However, as shown in FIG. 29, the distal tip of the removal device 2052 can include both a distal tip proximal portion 2046 and a distal tip distal portion 2047, connected to each other by a pin 2050 passing through lateral holes in each of the distal tip proximal portion 2046 and the distal tip distal portion 2047. Similar to the distal tip 2020 shown in FIG. 23, the distal tip proximal portion 2046 can be fixedly attached to the distal end of the outer tube 2018. FIG. 29 illustrates the outer tube 2018 with a proximal hub 2016 and proximal tube 2015 coupled to its proximal end and the distal tip proximal portion 2046 coupled to its distal end, e.g., without a distal tip distal portion 2047. The pin 2050 passing through the lateral holes in the distal tip proximal portion 2046 and the distal tip distal portion 2047 can be configured to allow the distal tip distal portion 2047 to pivot with respect to the distal tip proximal portion 2046. In some embodiments, the distal tip distal portion 2047 pivots substantially vertically with respect to the distal tip proximal portion 2046, e.g., due to the pin 2050 passing through the distal tip distal portion 2047 and the distal tip proximal portion 2046 in a substantially horizontal manner. Similar to the distal tip 2020 shown in FIG. 23, the distal tip distal portion 2047 can have a pair of wings with one or more holes 2051 configured to rotatably or pivotably hold a pair of jaws, e.g., jaw 2048 and jaw 2049.

In some embodiments, a single control wire (which can also be referred to herein as a pull wire) 2045 can be used to control both deflection of the distal tip distal portion 2047 with respect to the distal tip proximal portion 2046 as well as opening and closing of the pair of jaws, e.g., the jaw 2048 and jaw 2049. The use of a single control wire 2045 can advantageously facilitate single handed operation by a user.

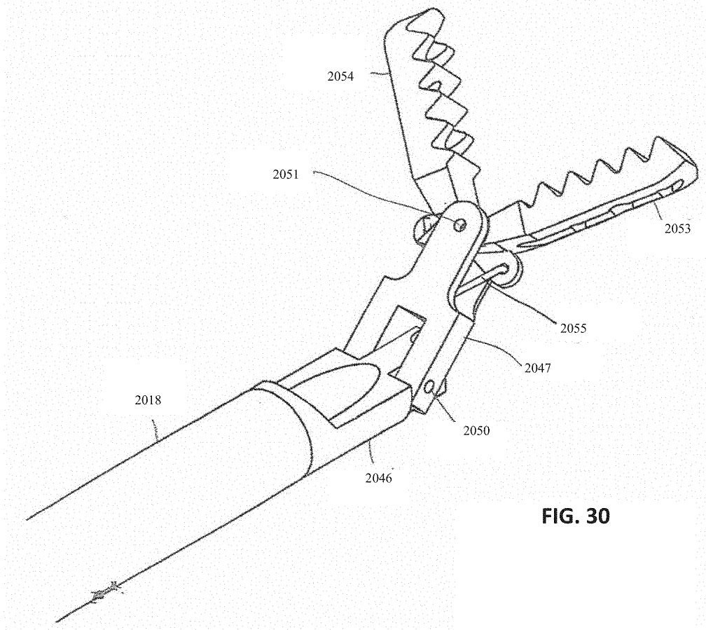

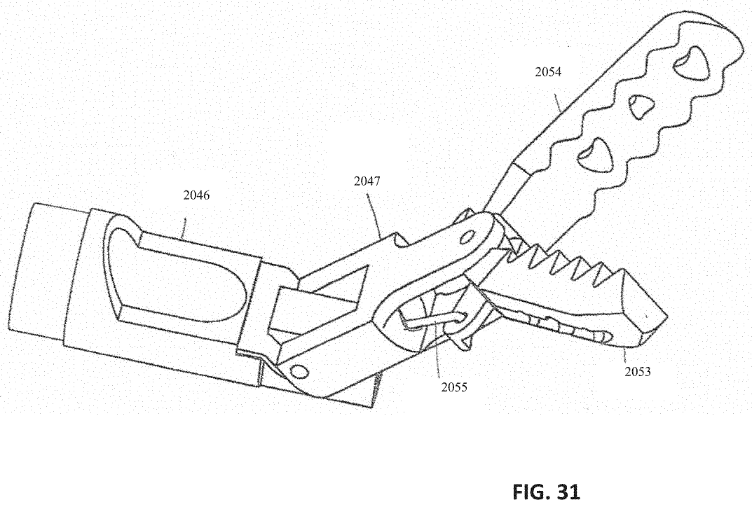

FIG. 30 illustrates an outer tube 2018 to which a distal tip proximal portion 2046 is fixed. A distal tip distal portion 2047 holding a pair of jaws, e.g., jaw 2053 and jaw 2054 (e.g., using a pin passing through the holes 2051), is attached to the distal end of the distal tip proximal portion 2046 using a hinging or pivotable attachment, e.g., using pin 2050 passing through holes in each of the distal tip proximal portion 2046 and the distal tip distal portion 2047. Similar to as was described in connection with the removal device 2001 of FIG. 23, the removal device 2052 of FIG. 30 includes a control wire 2045 that passes through at least a portion of the outer tube 2018, through at least a portion of the distal tip proximal portion 2046, and through at least a portion of the distal tip distal portion 2047 (e.g., as shown, the control wire 2045 passes at least through a portion of the wings or tabs of the distal tip distal portion 2047), and ultimately attached directly or indirectly to the jaws, e.g., using one or more control wire jaw attachment 2055. In this way, pulling distally on the control wire (or pull wire) 2045 can cause the jaws to close, while pushing proximally on the control wire 2045 can cause the jaws to open. FIG. 31 illustrates the distal end of the removal device 2052 shown in FIG. 30, from a vertical side angle. As can be seen more clearly in FIG. 31, the distal tip distal portion 2047 can have a longitudinal bore, communicating with a bore in the distal tip distal portion 2047 through which the control wire 2045 can pass. At least the control wire jaw attachment 2055 exits the bore of the distal tip distal portion 2047 where it attaches to the jaw 2053 and jaw 2054. FIG. 31 illustrates the distal tip distal portion 2047 deflected an angular distance, e.g., deflected vertically, with respect to the distal tip proximal portion 2046. As described in FIGS. 9(a), 9(b), 45, 46, and 47, in some embodiments, the jaws 2053, 2054 can have surface damaging structures, such as, hollow teeth, hollow needles, or cannulated needles, and puncturing members.

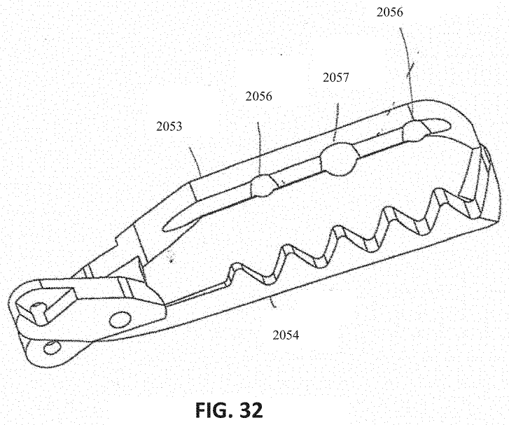

FIG. 32 illustrates an enlarged view of the jaw 2053 and jaw 2054, pivotably attached to each other by a pin passing through a hole in each of the jaw 2053 and the jaw 2054. As can be seen, the jaw 2053 includes an external vent channel (also called a venting groove) 2057 positioned parallel to the axis of the jaw 2053. The external vent channel 2057 or venting groove is configured to allow venting from a balloon, or inflatable implant, as well as any liquid proximal to the jaws to escape to the bladder through sheath vent holes, as described further herein. In some embodiments, one or more needle vent holes 2056 or transverse vent holes communicate with the external vent channel 2057 or venting groove, allowing air or any other fluid to pass from the needles held by the jaws, through the needles and out the back of the jaw where it can be channeled by the external vent channel 2057 or venting groove. The needle vent holes can be in communication with a surface damaging structures, such as, hollow teeth, hollow needles, or cannulated needles, and puncturing members

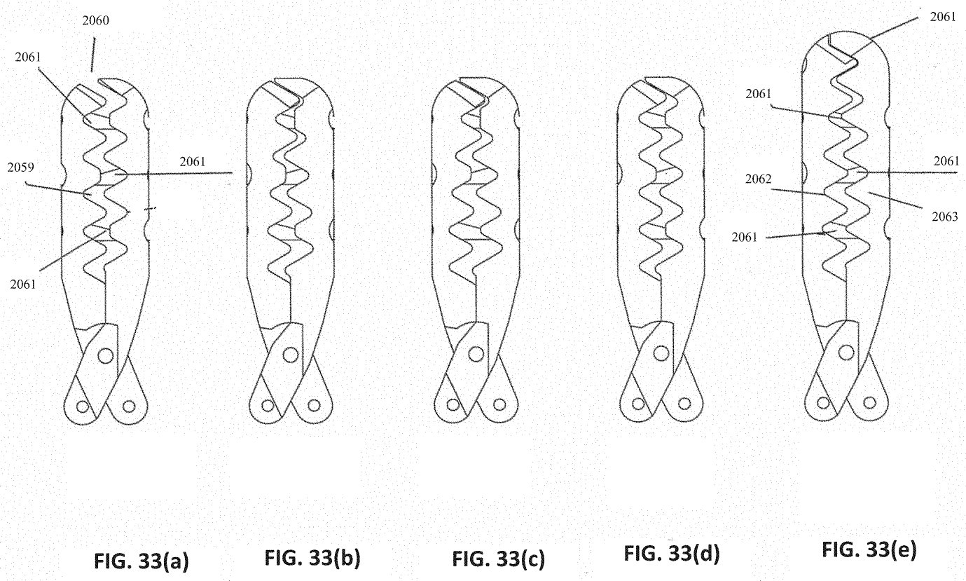

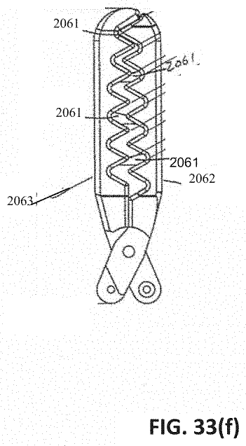

FIGS. 33(a), 33(b), 33(c), 33(d), 33(e), and FIG. 33(f) illustrate various jaw configurations that can be used in connection with one or more of the removal devices disclosed herein. FIG. 33(a) illustrates an open full length embodiment with a pair of jaws, e.g., jaw 2058 and jaw 2059, attached at a pivot point. The pair of jaws shown in FIG. 33(a) have three needle 2061, e.g., two needles 2061 attached to the jaw 2058 and one needle 2061 attached to the jaw 2058 and a tooth gap 2060. The needles 2061 can be hollow needles or cannulated needles as described above. As shown, the tooth gap 2060 in FIG. 33(a) remains substantially constant from the tip of the jaws to the rear of the jaws (beginning with an initial tooth gap of about 0.023 inches). FIG. 33(b) illustrates an tapered opening embodiment with a pair of jaws, e.g., jaw 2058 and jaw 2059, similar to the jaws of FIG. 33(a), except the tooth gap 2060 is tapered (these can be termed tapered opening jaws). As shown, the initial tooth gap is about 0.003 inches. Then, each successive inter-tooth distance is about 0.006 inches, about 0.012 inches, about 0.018 inches, about 0.024 inches, and about 0.024 inches, respectively. FIG. 33(b) illustrates another embodiment of tapered opening jaws, having a different taper. As shown, by contrast to the tapered opening jaws of FIG. 33(b), the initial tooth gap is about 0.003 inches and each successive inter-tooth distance is about 0.006 inches, about 0.018 inches, about 0.026 inches, about 0.032 inches, and about 0.032 inches, respectively. FIG. 33(c) illustrates a tapered opening embodiment. FIG. 33(d) illustrates a open at center embodiment with a pair of jaws configured in a similar fashion to the jaws of FIG. 33(a), except where the jaws in FIG. 33(a) have an open initial tooth gap, e.g., an initial tooth gap of about 0.023 inches, the jaws of FIG. 33(d) have a much smaller or closed initial tooth gap, e.g., an initial tooth gap of only about 0.003 inches. Finally, FIG. 33(e) illustrates an tapered opening medium length embodiment with a longer set of jaws, having a tapered opening, similar to that shown in FIG. 33(b) or FIG. 33(c), but having a longer proximal to distal length. Additionally, the longer law length can accommodate additional teeth and additional needles 2061: as shown, the jaws accommodate 4 needles 2061, with two needles 2061 in the jaw 2058 and two needles 2061 in the jaw 2059. FIG. 33(f) illustrates another embodiment of a pair of jaw having a tapered opening, similar to that shown in FIGS. 33(b) and 33(c). The pair of jaws shown in FIG. 33(f) includes a jaw 2062 and a 2063 having a proximal to distal length of about 0.85 inches. Of course, any other length of jaws can be used. For example, the pair of jaws can have a proximal to distal length of between about 0.1-3 inches, about 0.2-2.8 inches, about 0.3-2.6 inches, about 0.4 2.4 inches, about 0.5-2.2 inches, about 0.6-2 inches, about 0.7-1.8 inches, about 0.8-1.6 inches, about 0.9-1.4 inches, or about 1-1.2 inches. The pair of jaws can have an initial tooth gap of about 0.002 inches and each successive inter-tooth distance is about 0.012 inches, about 0.017 inches, about 0.022 inches, about 0.024 inches, and about 0.026 inches, respectively. The jaws shown in FIG. 33(f) include four needles 2061, two on each jaw, in a staggered format.

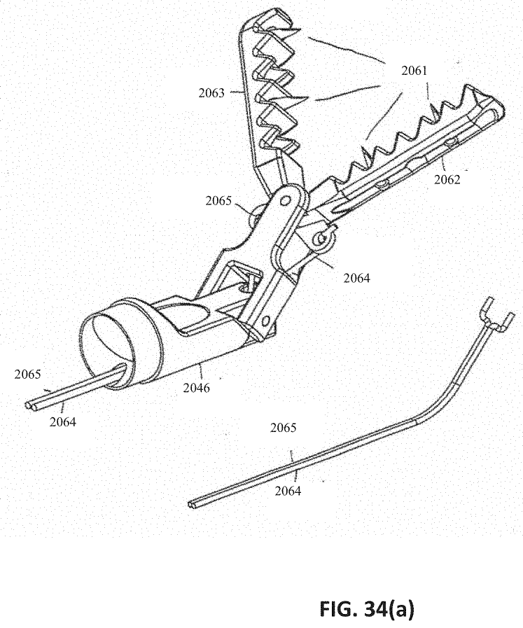

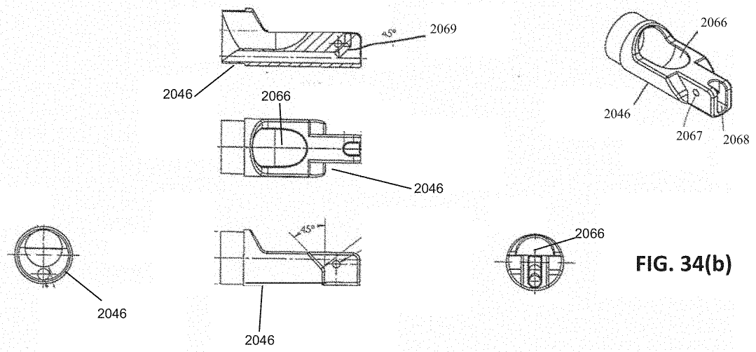

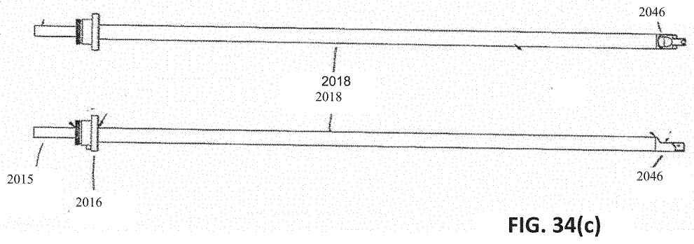

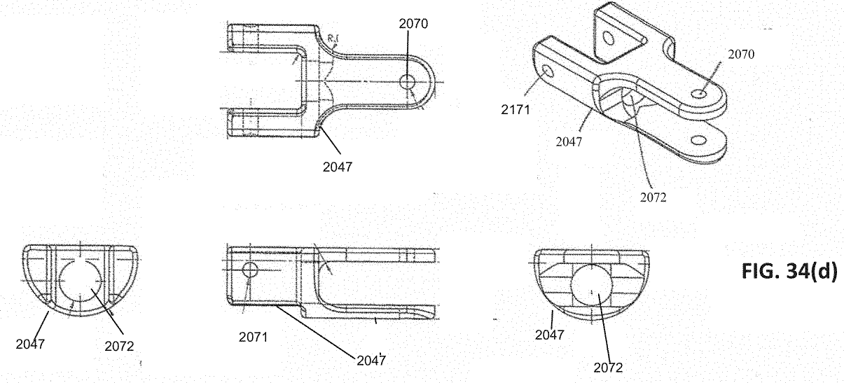

As shown in FIG. 34(c), distal tip proximal portion 2046 can be attached, e.g., welded, to the distal end of the outer tube 2018. The distal tip proximal portion 2046 is shown in additional detail in FIG. 34(b). Similar to the distal tip 2020 discussed in connection with FIG. 23, the distal tip proximal portion 2046 can include a scoop 2066, similar to the scoop 2026. The scoop 2066 can comprise a continuous curved surface that is opposite the opening of the outer tube 2018 for the scope 2000, so as to minimize direct reflection of light from the scope. In addition, the distal tip proximal portion 2046 can include a longitudinal passage 2068, e.g., below the scoop 2066, to permit that pull wires, e.g., control wire 2045 to extend through the distal tip proximal portion 2046. The longitudinal passage 2068 can include, e.g., terminate in an edge 2069, e.g., an angled edge, configured to permit the control wire 2045 to deflect. Various parameters of the edge 2069 can be altered to change one or more characteristics of deflection of the distal tip distal portion 2047 when the control wire 2045 is moved proximally or distally, e.g., the magnitude, speed, etc. For example, the edge 2069 can be positioned more proximally or more distally in the distal tip proximal portion 2046. The edge 2069 can be radiused with a larger or smaller radius. The edge 2069 can have an angle of about 45 degrees, an angle of greater than about 45 degrees, or an angle of less than about 45 degrees. As discussed herein, the distal tip proximal portion 2046 has a hole 2067 that can be used to hingedly or pivotably couple or attach the distal tip distal portion 2047 to the distal tip proximal portion 2046.

FIG. 34(d) illustrates an embodiment of the distal tip distal portion 2047. The 2071 of the distal tip distal portion 2047 can be used to pivotably couple the distal tip distal portion 2047 to the distal tip proximal portion 2046 using a pin inserted through both the 2071 and the hole 2067, when the two are aligned. In this way, the distal tip distal portion 2047 can pivot with respect to the distal tip proximal portion 2046 around the hole 2067. As shown, the distal tip distal portion 2047, like the distal tip 2020 of FIG. 23, includes a pair of wings or tabs, which have holes 2070 that can be used to connect a pair of jaws to the distal tip distal portion 2047. In addition, the distal tip distal portion 2047 can include a pass through 2072 configured to allow passage of pull wire(s), e.g., control wire 2045. In this way, pull wires can travel through the outer tube 2018, through the passage 2068 of the distal tip proximal portion 2046, and through the pass through 2072 of the distal tip distal portion 2047 to reach the jaws.

FIG. 34(a) illustrates an embodiment of a set of jaws, installed in a distal tip proximal portion 2046 and a distal tip distal portion 2047, as discussed herein. The embodiment shown in FIG. 34(a) includes a distal tip proximal portion 2046 and a distal tip distal portion 2047 as disclosed herein. A jaw 2062 having two needles 2061 and a jaw 2063 having another two needles 2061 are held by the distal tip distal portion 2047, e.g., a pin is passed through the holes 2070 of the wings or tabs of the distal tip distal portion 2047 as well as corresponding holes in each of the jaw 2062 and the jaw 2063. The needles 2061 can be hollow needles, or cannulated needles. In addition, an external vent channel (also called a venting groove and not labeled in FIG. 34(a)) can be positioned parallel to the axis of the jaws 2063, 2062. As noted above with respect to FIG. 32, the external vent channel or venting groove can be configured to allow venting from a balloon, or inflatable implant, as well as any liquid proximal to the jaws to escape to the bladder through sheath vent holes, as described further herein. In some embodiments, one or more needle vent holes (not labeled in FIG. 34(a)) or transverse vent holes communicate with the external vent channel or venting groove. The transverse vent holes can be in communication with the cannulated needles or hollow needles of the jaw, 2062, 2063. The proximal end of each of the jaw 2063 and the jaw 2062 includes a hole configured for attachment of the pull wires. The embodiment shown in FIG. 34(a) incorporates two pull wires, including a first wire 2064 and a second wire 2065. Each pull wire can have a kink, bend or other attachment at its distal end configured to couple the wire to its respective jaw, e.g., one of the jaw 2062 and the jaw 2063. As shown in FIG. 34(a), both the first wire 2064 and the second wire 2065 enter the passage 2068 of the distal tip proximal portion 2046 at the proximal end of the distal tip proximal portion 2046, exit the passage 2068 of the passage 2068, e.g., at the location of the edge 2069, and enter the pass through 2072 of the distal tip distal portion 2047 where the first wire 2064 couples to the hole in the proximal end of the jaw 2063 and the second wire 2065 couples to the hole in the proximal end of the jaw 2062. This configuration allows both vertical deflection of the jaws (as shown) and opening and closing of the jaws (as shown, in their open configuration), by manipulating, e.g., advancing or retracting, the second wire 2065 and the first wire 2064. In some embodiments, the first wire 2064 and the second wire 2065 can be manipulated independently. In some embodiments, the first wire 2064 and the second wire 2065 and fixed to each other such that they can be manipulated as a pair.

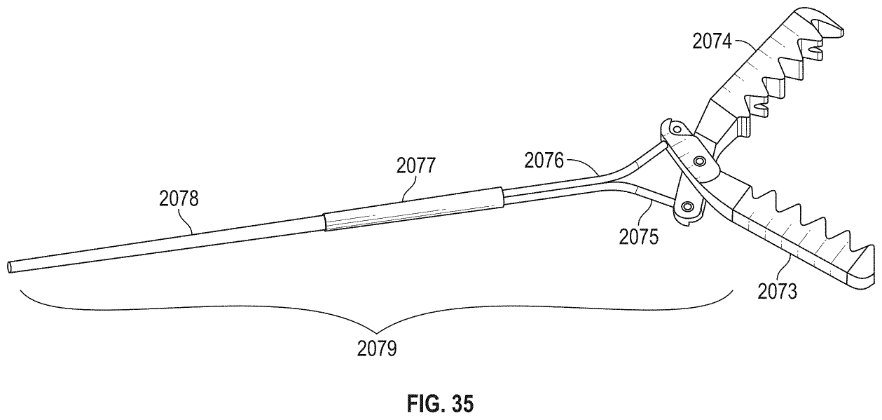

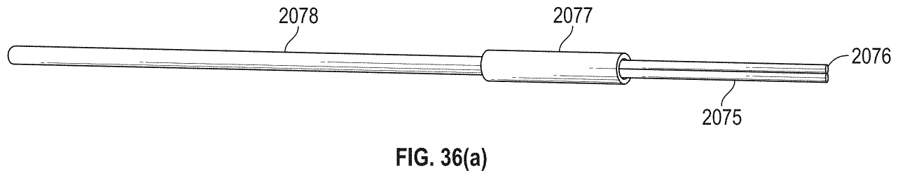

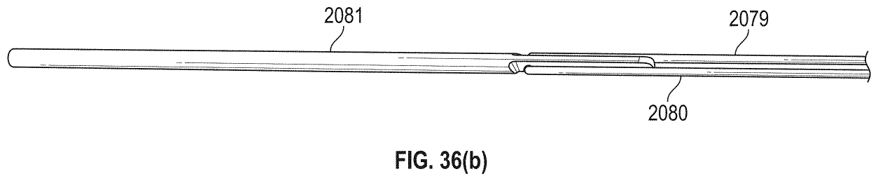

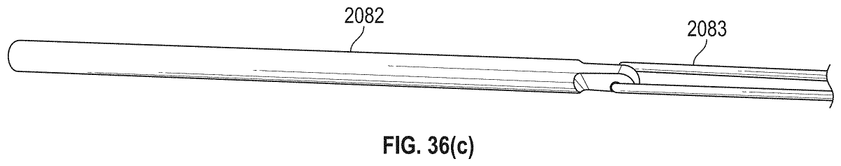

FIG. 35 illustrates an embodiment of a jaw assembly coupled to pull wires. As shown, the jaws, e.g., jaw 2073 and jaw 2074, are attached to a pull wire assembly 2079. The pull wire assembly 2079 includes a single pull wire 2078 coupled to two separate distal pull wires, e.g., distal pull wire 2075 and distal pull wire 2076, using a tube 2077. While any type of connection can be used, the pull wire 2078 can be connected to the distal pull wire 2076 by welding or fixing in any other fashion all three wires inside the tube 2077. The distal pull wire 2076 then travels distally to couple to a hole in the proximal end of the jaw 2073 and the distal pull wire 2075 then travels distally to couple to a hole in the proximal end of the jaw 2074. FIGS. 36(a), 36(b), 36(c), 37(a), and 37(b) illustrate various other pull wire configurations that can be used. FIG. 36(a) illustrates a configuration similar to that shown in FIG. 35, in which the pull wire 2078, distal pull wire 2075, and distal pull wire 2076 are all welded inside the tube 2077. FIG. 36(b) illustrates a machined pull wire 2081 terminating at its distal end in a machined tongue, e.g., a vertically oriented thinner segment having substantially flat sides. Each of the distal pull wire 2075 and distal pull wire 2076 can be welded or otherwise fixed to the machined tongue. FIG. 36(c) illustrates a machined pull wire 2082 terminating at its distal end in a machined tongue, e.g., a vertically oriented thinner segment having substantially flat sides and a transverse through hole. As shown, the distal pull wire 2075 and distal pull wire 2076 can be a single pull wire, passed or threaded through the transverse hole in the machined tongue of the machined pull wire 2082. FIGS. 37(a) and 37(b) illustrate additional embodiments of pull wires that can be used with various removal devices disclosed herein. As shown, the pull wire systems shown in FIGS. 37(a)-37(b) generally include a 218 connected to a distal pull wire 2075 and a distal pull wire 2076, e.g., using a tube 2077. FIGS. 37(c) illustrate further embodiments of pull wire systems that can be used with removal devices disclosed herein.

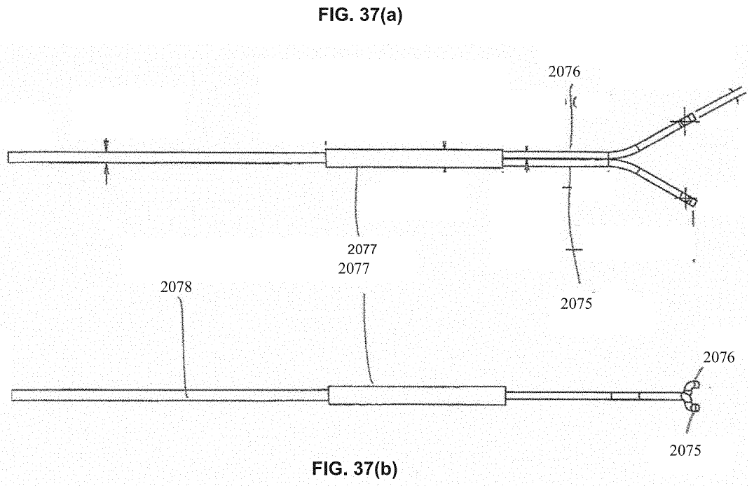

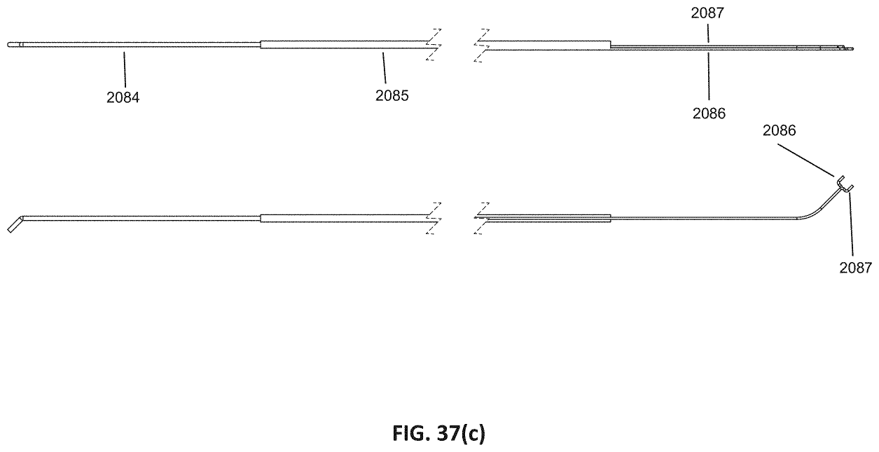

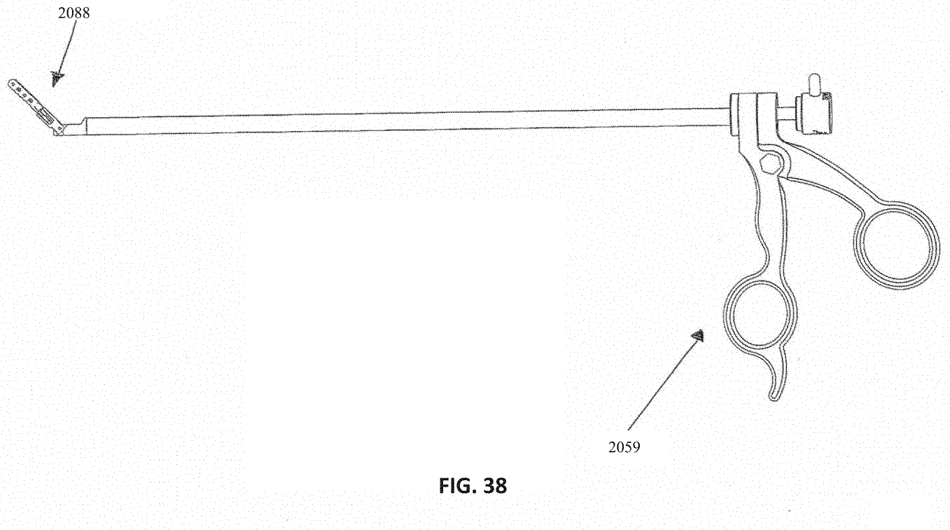

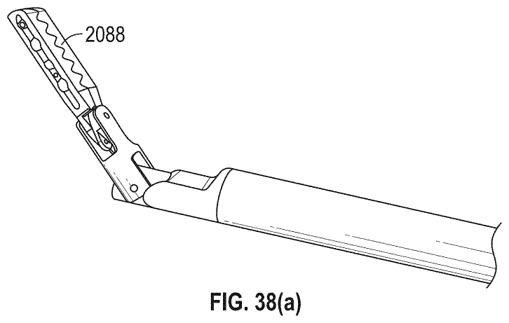

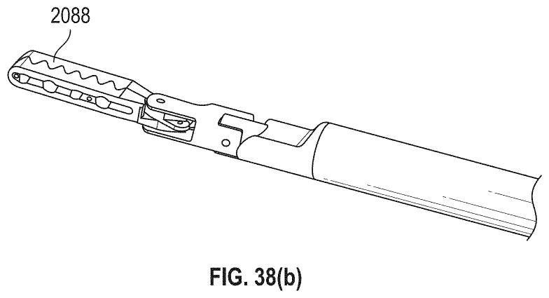

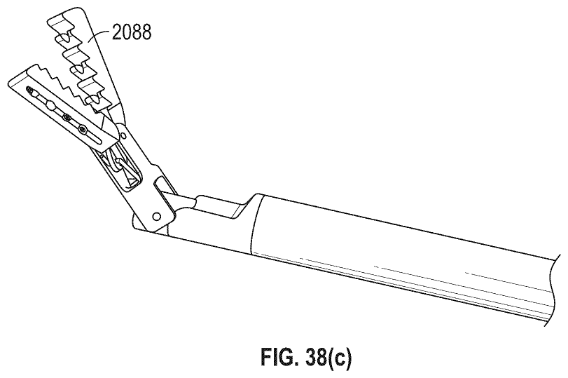

The pull wire systems as disclosed herein can be pre-formed, e.g., with specific lengths, flexibilities, curvatures, etc., and used to provide the desired deflection and actuation of the jaws. For example, the wires that attach to each jaw, e.g., the distal pull wire 2075 and the distal pull wire 2076 that attach to the holes in the proximal most portions of the jaws, e.g., the jaw 2063 and first wire 2064, can be pre-formed in a variety of shapes and lengths to obtain the appropriate timing and actuation of the deflection and jaw opening and/or closing. In some embodiments, the jaws are deflected in the resting position, e.g., when the handle is not manipulated, as shown in FIG. 38(a) (note that the rings of each handle are neither spaced from each other as far as possible nor are they as close to each other as possible (e.g., they are near or at a midpoint). In some embodiments, the jaws remain closed and there is no deflection when the handles are squeezed, as shown in FIG. 38(b) (note that the rings of each handle are spaced away from each other as far as possible, thereby pulling the pull wires proximally). In some embodiments, the jaws both deflect and open when the handle is opened, as shown in FIG. 3 8(c) (note that the rings of each handle are as close to each other as possible).

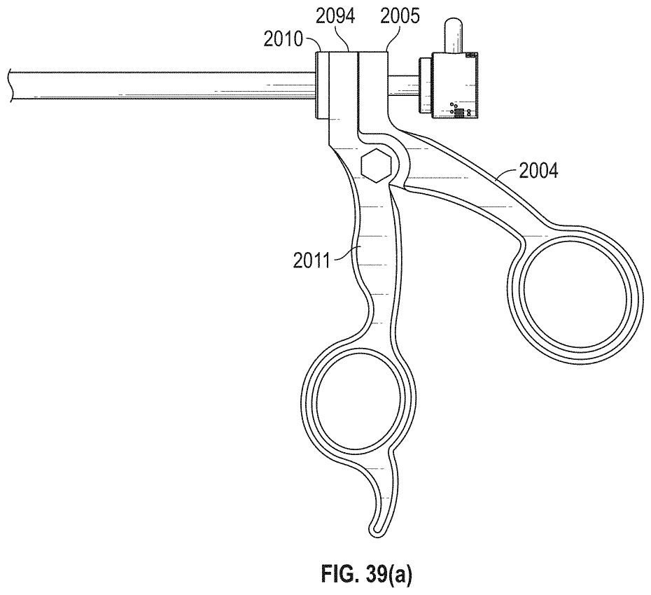

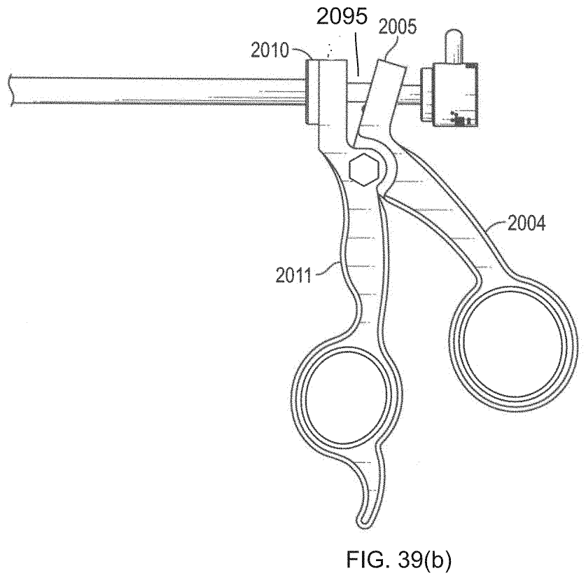

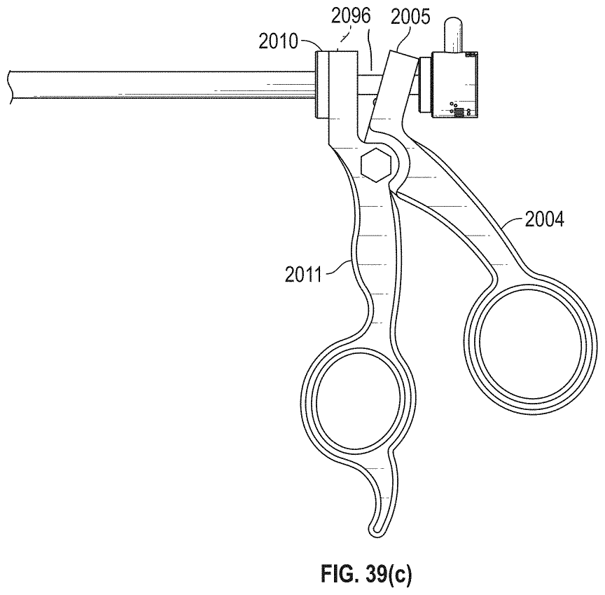

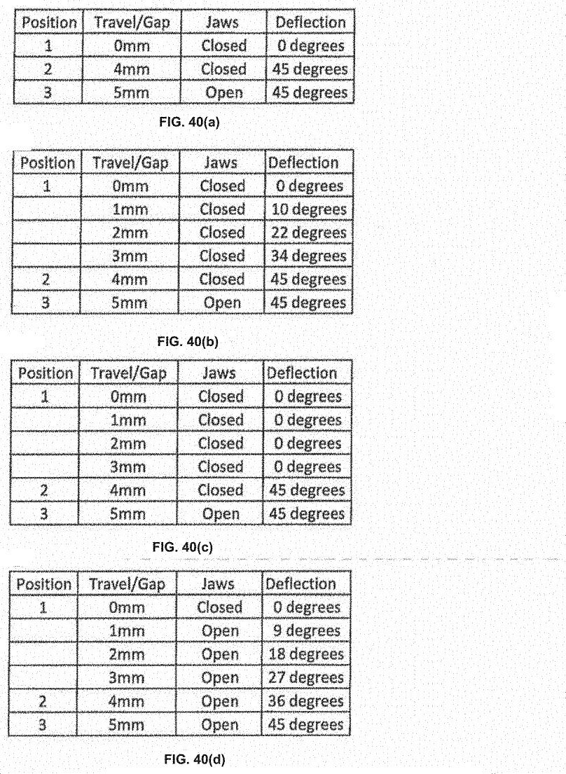

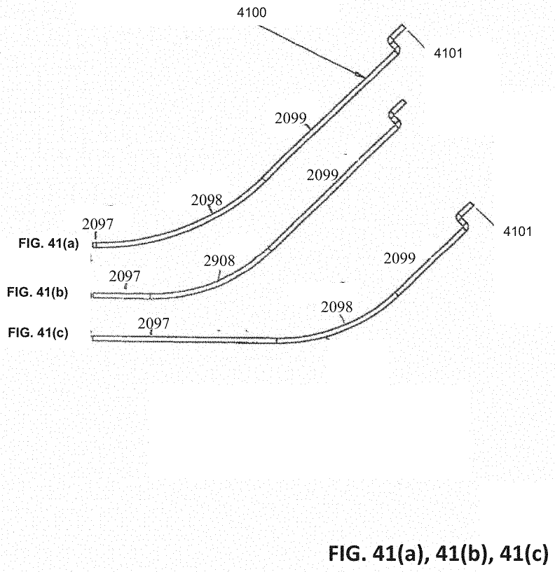

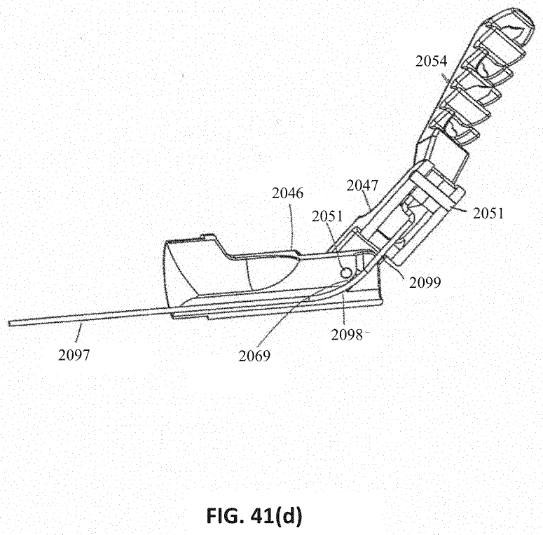

The three positions of 1) closed with deflection (FIG. 38(a) and FIG. 39(b)), 2) closed with no deflection (FIG. 38(b) and FIG. 39(c)), and 3) open with deflection can be evaluated by the movement of the pull wire (FIG. 38(c) and FIG. 39(a), as evidenced by the gap between the rings of the respective handles, e.g., moving handle ring 2005 of the moving handle 2004 and the handle ring 2010 of the handle 2011. The system can be tuned such that when the gap between the fixed handle ring 2010 and the moving handle ring 2005 is negligible, e.g., about 0 mm 2094, the jaws are deflected and open (e.g., position 1), such as is shown in FIG. 39(a). The system can be tuned such that when the gap between the fixed handle ring 2010 and the moving handle ring 2005 is about 4 mm 2095, the jaws are deflected but closed (e.g., position 2), such as is shown in FIG. 39(b). The system can be tuned such that when the gap between the fixed handle ring 2010 and the moving handle ring 2005 is about 5 mm 2096, the jaws are not deflected and are closed (e.g., position 3), such as is shown in FIG. 39(C). FIGS. 41(a), 41(b), and 41(c) show a change in radius can be used on the pull wire to control deflection speed. FIGS. 40(a), 40(b), 40(c), and 40(d) illustrate different possible configurations and jaw positions, e.g., gaps between the rings (or pull wire advancement/retraction) correlated to jaw position (deflection and open/closed). As shown in FIG. 40(a), the deflection of the jaws can occur abruptly from the 0 mm gap at 0 degrees to 4 mm gap at 45 degrees deflection and the jaws can open at the 5 mm gap. As shown in FIG. 40(b), the deflection of the jaws can occur gradually between the 0 and 4 mm gap and the jaws can open at the 5 mm gap. As shown in FIG. 40(c), the deflection of the jaws can occur abruptly from 0 at 3 mm to 45 degrees at 4 mm and the jaws can open at the 5 mm gap. As shown in FIG. 40(d), the deflection of the jaws can occur gradually over the entire 5 mm while the jaws open at a 1 mm gap. Such scenarios, e.g., advancement or retraction of the pull wire(s) necessary to deflect and/or open/close the jaws, can be programmed into the removal device by varying one or more characteristics of the pull wires, e.g., length and/or curvature of one or more portions, and the shape of the interface on the edge 2069 of the distal tip proximal portion 2046 (e.g., as shown in FIG. 41(d)), among other possible factors. As shown in FIG. 41(a), with the jaws closed and not deflected, the opening of the jaws will occur immediately upon actuation of the handle followed by the deflection of the jaws. As shown in FIG. 41(b), with the jaws closed and not deflected, the deflection of the jaws and the opening of the jaws may occur simultaneously. As shown in FIG. 41(c), with the jaws closed and not deflected, the deflection of the jaws can occur immediately and be followed by the opening of the jaws.

FIGS. 41(a), 41(b) and 41(c) illustrate different pull wire configurations that can change the magnitude of pull wire advancement and retraction needed to open/close and/or deflect the jaws. Each of the pull wires has three sections or zones to the pull wire shape, including an axial section 2097, a curved section 2098, and a neck section 2099. The axial section 2097 is a straight portion that extends proximally into the outer tube 2018 and determines when the wires deflect when traveling out of the distal tip proximal portion 2046 against the edge 2069. As can be seen, the axial section 2097 is shortest in FIG. 41(a) and longest in FIG. 41(c). The curved section 2098 has a radius of curvature that can change the rate of speed that the jaws deflect, e.g., the smaller the curvature in the curved section 2098, the faster the jaws will deflect whereas the greater radius of curvature in the curved section 2098, the slower the jaws will deflect. As can be seen, FIG. 41(a) has a long curved section 2098 with a medium radius of curvature; FIG. 41(b) has a shorter curved section 2098 with a smaller radius of curvature; and FIG. 41(c) has a shorter curved section 2098 with a greater radius of curvature. Finally, the neck section 2099 has a length that determines when the jaws open and how far the jaws open relative to the deflection. The parameters of each of these sections, e.g., length and curvature, can be tailored, e.g., increased or decrease, to change the degree or timing of jaw deflection and/or jaw opening/closing. FIG. 41(d) illustrates a cross-sectional view of a distal tip proximal portion 2046, distal tip distal portion 2047 and jaw 2054 in relation to the various sections of a pull wire. As can be seen, the axial section 2097 extends into the passage 2068 of the distal tip proximal portion 2046. The curved section 2098 of the pull wire is around the edge 2069 of the distal tip proximal portion 2046 so that the two can interact and provide deflection of the jaw, as is shown. Finally the neck section 2099 extends past the edge 2069, through the pass through 2072 of the distal tip distal portion 2047 where it attaches to the proximal end of the jaw 2054. Additional bends can be place in the distal area of the pull wire 4100 to control jaw closing speed and the location of the J hook 4101 controls the timing of the jaw closure in relation to the direction.

In some embodiments, the removal devices disclosed herein incorporate more than one pull wire. For example, some embodiments include two pull wires, a first pull wire for controlling the deflection of the jaws and a second pull wire for controlling the opening and closing of the jaws. Thus, it can be possible to have various combinations of the following configurations: jaws closed with no deflection; jaws closed with deflection; jaws opened with no deflection; and jaws open with deflection.

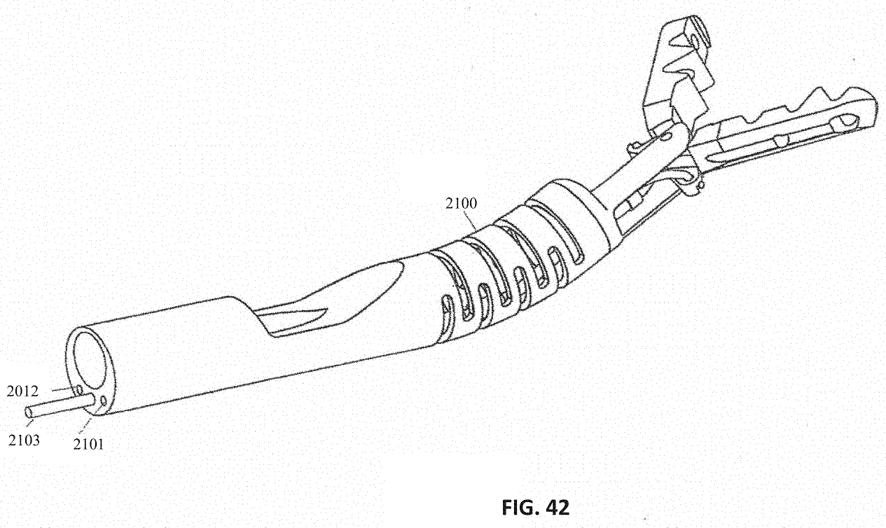

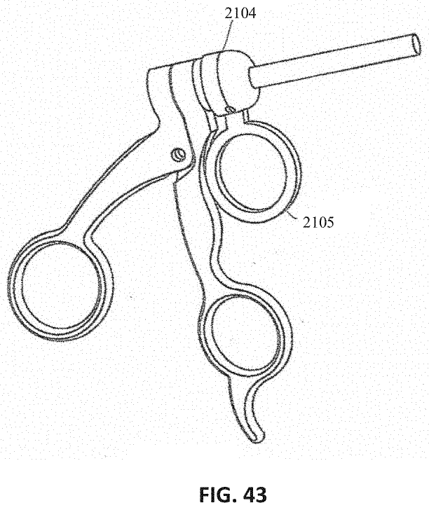

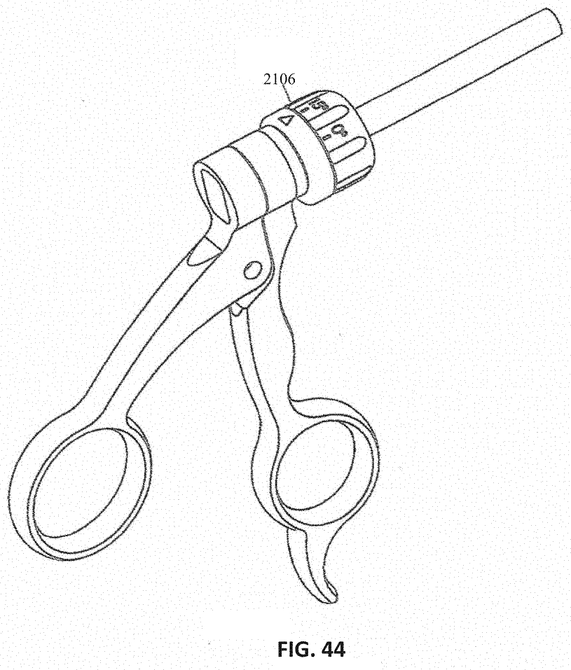

FIG. 42 illustrates the distal end of an embodiment of a removal device that is configured to maintain the lateral opening of the jaws while permitting deflection of the jaws in any direction. The removal device 2100 includes at least two control wires (also can be referred to as a pull wires), one control wire for opening and closing the jaws and at least one control wire for deflecting the jaws. In this way, the at least one control wire for deflecting the jaws can be used to deflect the jaw in any direction. FIGS. 43 and 44 illustrate various handles that can be used in connection with removal devices having more than one control wire. FIG. 43 illustrates a handle similar to that shown in FIG. 23 with the addition of a second movable handle 2105 that is configured to move a second moving handle ring 2104 to axially move a second (or other) control wire. FIG. 44 illustrates a handle similar to that shown in FIG. 23 with the addition of a rotatable dial configured to axially move a second (or other) control wire. As shown in FIG. 44, the rotatable dial can be provided with markings indicating the degree of deflection provided by the dial. For example, rotation of the rotatable dial in a counter clockwise direction increases deflection of the jaws while rotation of the rotatable dial in a clockwise direction decreases deflection of the jaws.

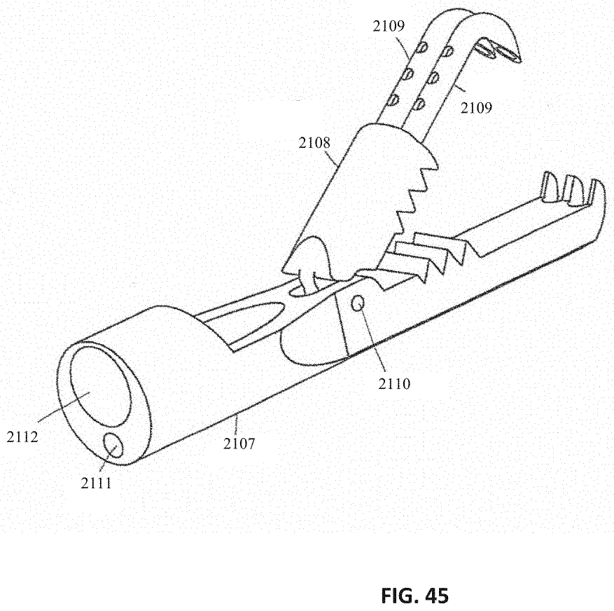

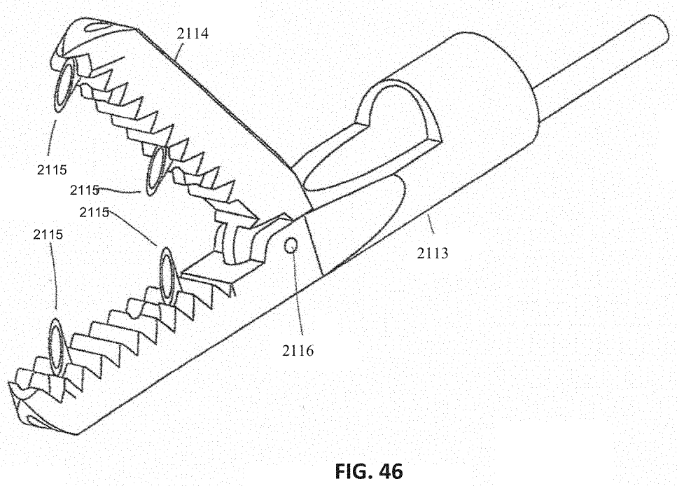

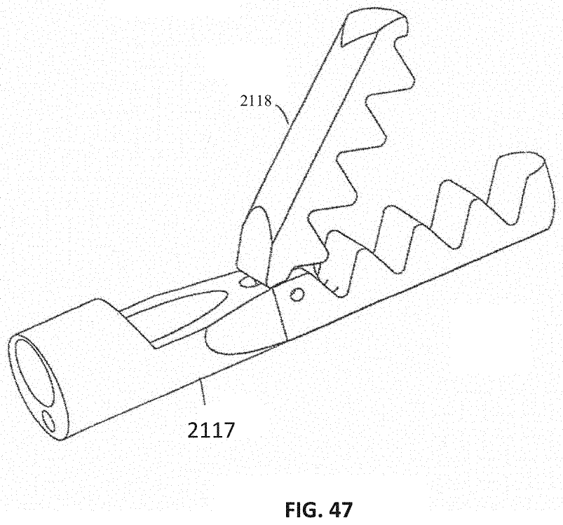

FIGS. 45, 46, and 47 illustrate various embodiments of jaws that open vertically, rather than laterally. Each of the jaws shown in FIGS. 45, 46, and 47 includes one fixed jaw, e.g., a lower jaw, and a movable jaw, e.g., an upper jaw. Therefore, such jaws cannot be deflectable. FIG. 45 illustrates a jaw assembly having an upper, rotatable jaw 2108 pivotably attached to a lower, fixed jaw 2107. The upper jaw 2108 can include one or more hollow teeth or fangs 2109 with vents on the outer surface (three vents are shown on each tooth or fang 2109). The jaw assembly of FIG. 45 includes a pass through 2112 configured to accept a scope as disclosed elsewhere herein and a pass through configured to accept a pull wire configured to open and close the jaw assembly. FIG. 46 illustrates a jaw assembly having an upper, rotatable jaw 2114 pivotably attached to a lower, fixed jaw 2113. The upper jaw 2114 has a number of teeth and needles 2115, e.g., hollow or cannulated needles. The lower jaw 2113 can also have a number of teeth and needles 2115, e.g., hollow or cannulated needles. As shown, the upper jaw 2114 has two needles 2115 and the lower jaw has two needles 2115. The needles 2115 can be configured to permit venting through the body of the cannulated needle and out the jaws (e.g., each jaw can have one or more ports on an outer surface of the jaw in communication with the one or more needles). FIG. 47 illustrates another embodiment of a jaw assembly. As shown, the jaw assembly includes an upper toothed jaw 2118 that is pivotably attached to a fixed lower toothed jaw 2117.

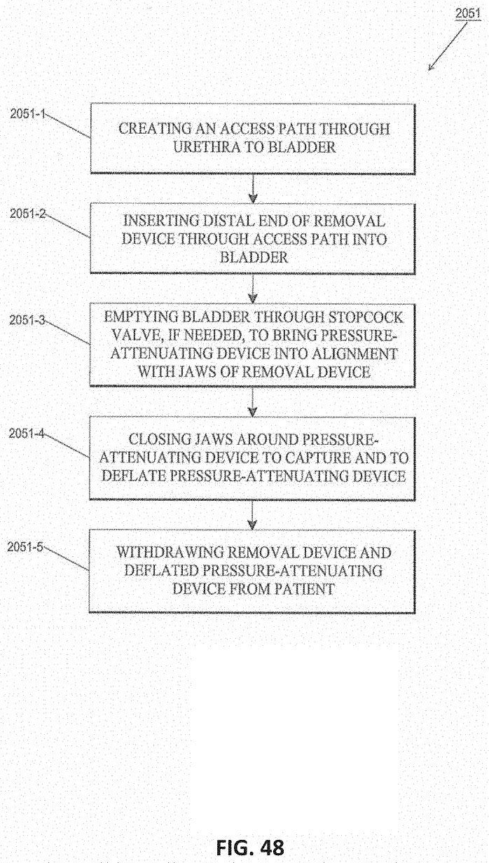

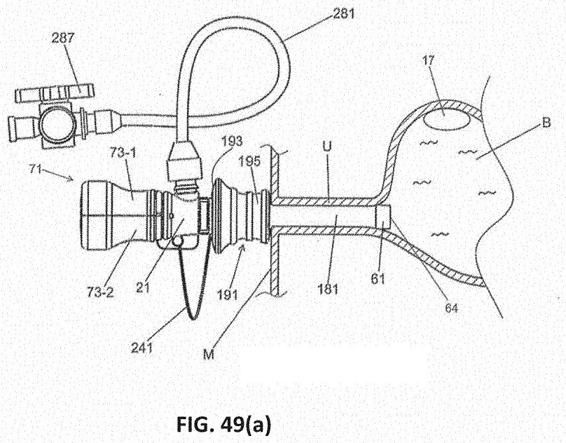

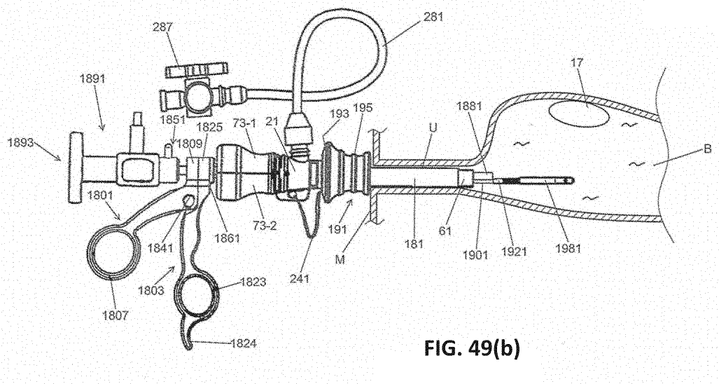

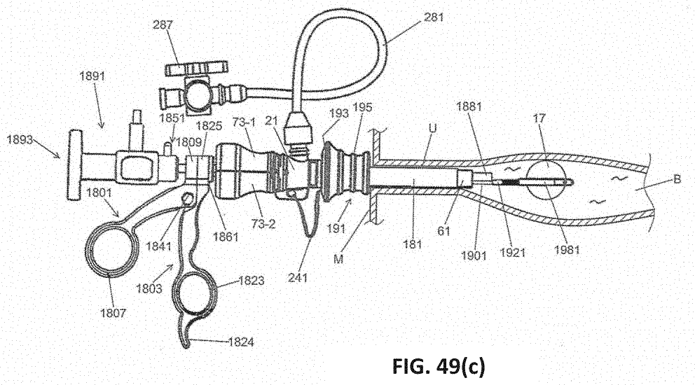

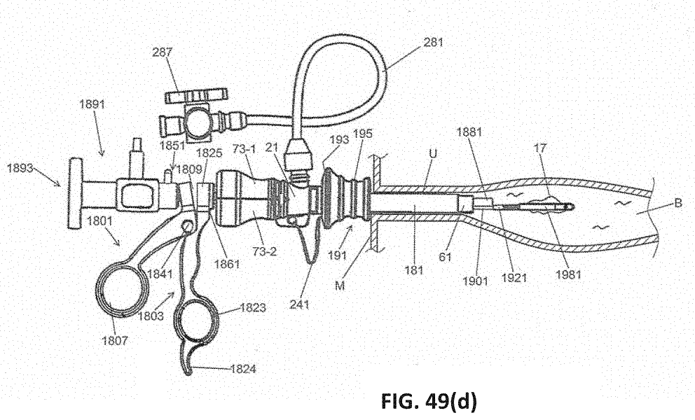

Referring now to FIG. 48, there is shown a flowchart, schematically depicting one possible method 2051 of using removal device 19 to remove an implanted pressure-attenuating device 17 from an anatomical structure of a patient, such as a bladder. Method 2051 can begin with a step 2051-1 of installing an access device in a patient in any of the manners discussed above. Where, for example, access device 13 is used to provide transurethral access to the bladder, said installing step can comprise inserting distal end 135 of obturator 131, which can be covered by sleeve 181, into the urethra, advancing obturator 131 and sheath 61 through the urethra and into the bladder, and then removing obturator 131, whereby an access path extending across the urethra and into the bladder can be created (see FIG. 49(a)). Method 2051 can then continue with a step 2051-2 of inserting the distal end of a removal device through the access device and into the anatomical structure of the patient. This can be done by inserting the distal end of removal device 19 through the remaining installed portion of access device 13 and into the bladder of the patient (see FIG. 49(b)). (In order to permit insertion of removal device 19 into access device 13, one can use one hand to pivot ring portion 1807 of member 1801 towards ring portion 1823 of member 1803 until jaws 1981 and 1983 close. After jaws 1981 and 1983 have been inserted completely through access device 13, jaws 1981 and 1983 can then be opened by pivoting ring portion 1807 away from ring portion 1823. Proper placement of the distal end of device 19 within the bladder can be confirmed by observation with scope 1891.)

Where the method 2051 is performed in the bladder, or other fluid filled structure, the method can then continue with a step 2051-3 of emptying the structure of liquid, such as through stopcock valve 287, until the inflated device comes into alignment with removal device. For example, urine can be removed from the bladder until the device 17 is aligned with opened jaws 1981 and 1983 as observed through scope 1891 (see FIG. 49(c)). Method 2051 can then continue with a step 2051-4 of engaging the inflated device with the removal device. This can also include deflating the inflated device. For example, the jaws 1981 and 1983 can close around device 17, causing device 17 to deflate over the next several seconds (see FIG. 49(d)). Method 2051 can then conclude with a step 2051-5 of withdrawing removal device, together with the deflated pressure-attenuating device from the anatomical structure through the access device. The implanted device 17 can be held between jaws 1981 and 1983 and can be removed through the remaining installed portion of access device 13 while the remaining installed portion of access device 13 is held stationary in the patient. If, for some reason, device 17 has not deflated completely as it is being withdrawn from the patient, the distal end 64 of sheath 61 can advantageously serve as a fulcrum to help to compress device 17 sufficiently for its facile withdrawal from the patient. (Access device 13 can thereafter be removed from the patient or can remain in the patient to provide a conduit through which observational, removal, or other devices can be inserted.)

Alternative embodiments to sheath 61 are shown in FIGS. 21 and 22 and are represented generally by reference numerals 2071 and 2081, respectively. Sheaths 2071 and 2081 can be similar in most respects to sheath 61, sheaths 2071 and 2081 differing principally from sheath 61 in that sheaths 2071 and 2081 can include distal ends 2073 and 2083, respectively. Distal ends 2073 and 2083 can be advantageous in increasing the contact surface area during removal of device 17 and in forcing device 17 in a certain direction during removal.

As can readily be appreciated, although removal device 19 is discussed above as being used for observation and removal of an implanted device 17, removal device 19 could alternatively be used solely for observation of an implanted device 17, for example, for observation of an implanted device 17 immediately after its implantation in a patient to confirm that device 17 has been implanted properly.

As can be seen from the above discussion, one desirable feature of removal device 19 is that removal device 19 can be operated with one hand.

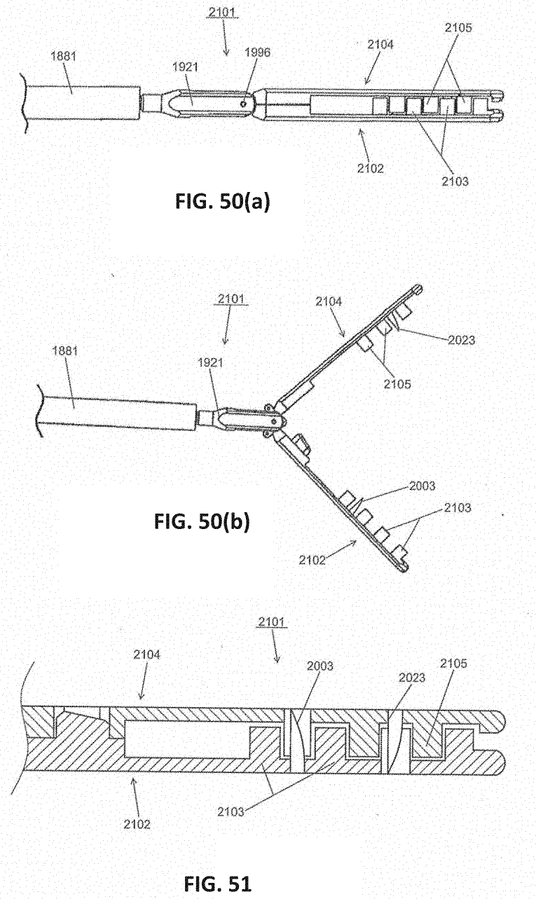

Referring now to FIGS. 50(a), 50(b), and 51, there are shown various views of a first alternate embodiment of a removal device, the first alternate embodiment of the removal device being represented generally by reference numeral 2101.

Removal device 2101 can be similar in many respects to removal device 19. A principal difference between the two devices can be that, whereas removal device 19 can comprise jaws 1981 and 1983 comprising rows of teeth 1997 and 2017, respectively, that can be generally triangular in shape in side profile (i.e., when viewed from above device 19), removal device 2101 can comprise jaws 2102 and 2104 comprising rows of teeth 2103 and 2105, respectively, that can be generally rectangular in shape in side profile.

Removal device 2101 can be used in a similar fashion to removal device 19.

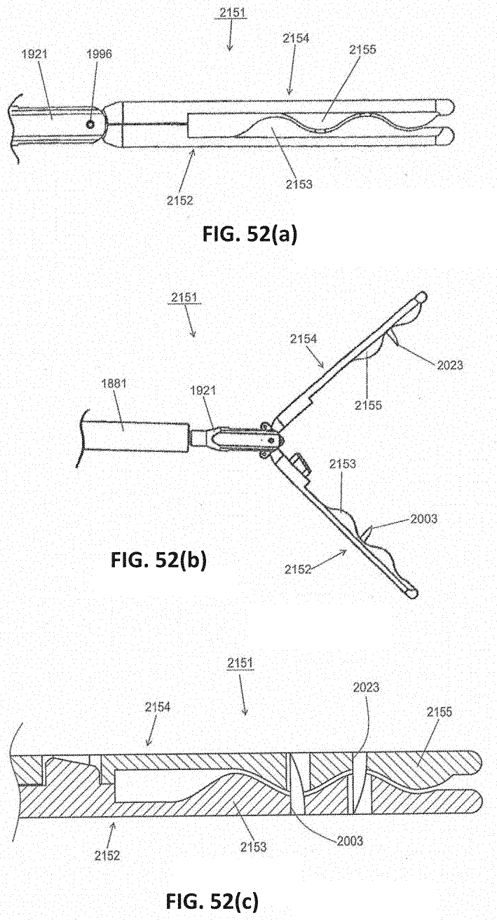

Referring now to FIGS. 52(a), 52(b), and 52(c), there are shown various views of a second alternate embodiment of a removal device, the first alternate embodiment of the removal device being represented generally by reference numeral 2151.

Removal device 2151 can be similar in many respects to removal device 19. A principal difference between the two devices can be that, whereas removal device 19 can comprise jaws 1981 and 1983 comprising rows of teeth 1997 and 2017, respectively, that can be generally triangular in shape in side profile (i.e., when viewed from above device 19), removal device 2151 can comprise jaws 2152 and 2154 comprising rows of teeth 2153 and 2155, respectively, that can be generally sinusoidal in shape in side profile.

Removal device 2151 can be used in a similar fashion to removal device 19.

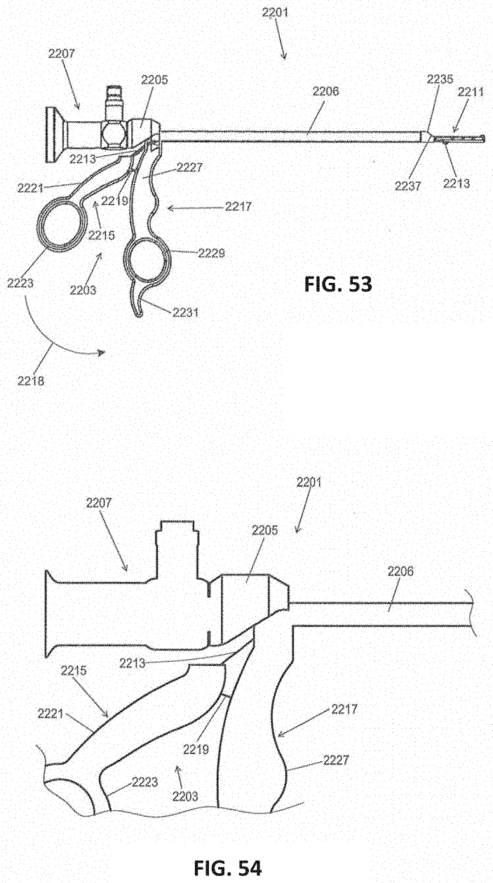

Referring now to FIGS. 53, 54, and 55(a) through 55(c), there are shown various views of a third alternate embodiment of a removal device, the third alternate embodiment of the removal device being represented generally by reference numeral 2201.

Removal device 2201, which can be similar in many respects to removal device 19, can comprise a scissors-like handle 2203, a hub 2205, a sheath 2206, a cystoscope 2207, a pair of jaws 2209 and 2211, a plurality of cannulated needles 2212-1 through 2212-3, and a wire 2213.

Scissors-like handle 2203, which can be a unitary structure made of a hard, medical-grade polymer or a similarly suitable material, can comprise a first member 2215, a second member 2217, and a living hinge member 2219. First member 2215 can be shaped to comprise an elongated arm portion 2221. A transversely-extending ring portion 2223, which can be appropriately dimensioned to receive, for example, the thumb of a user, can be disposed at one end of arm portion 2221. Second member 2217 can be shaped to comprise an elongated arm portion 2227. A transversely-extending ring portion 2229, which can be appropriately dimensioned to receive, for example, the forefinger of a user, and a finger rest 2231, which can be appropriately dimensioned to receive, for example, the middle finger of a user, can be disposed at one end of arm portion 2227. The opposite end of arm portion 2227 can be fixedly secured to sheath 2206. First member 2215 can be coupled to second member 2217 for pivotal movement relative thereto by living hinge member 2219. In this manner, handle 2203 can be operated much like a pair of scissors, albeit with first member 2215 being regarded as a movable member and with second member 2217 being regarded as a stationary member. It is to be understood, however, that handle 2203 could be modified so that both first member 2215 and second member 2217 are movable.

Hub 2205 can be a unitary, tubular structure made of a hard, medical-grade polymer or a similarly suitable material, and sheath 2206 can also be a unitary, tubular structure made of a hard, medical-grade polymer or a similarly suitable material. Hub 2205 and sheath 2206 can be joined to another by welding, adhesive or other suitable means and can be arranged to be coaxial with one another, with hub 2205 having a comparatively larger diameter and with sheath 2206 having a comparatively smaller diameter. Each of hub 2205 and sheath 2206 can be appropriately dimensioned to coaxially receive cystoscope 2207. Hub 2205 and sheath 2206 can have a combined length such that, when cystoscope 2207 is fully inserted into hub 2205 and sheath 2206, a distal end 2235 of cystoscope 2207 can extend just distally beyond a distal end 2237 of sheath 2206.

Sheath 2206 can be circular in transverse cross-section, which can be advantageous in helping to form a tight seal, for example, with seal 125 of access device 13 or, for example, with seal 917 of access device 901.

Cystoscope 2207 can be identical in size, shape, construction, and function to cystoscope 1891 of removal device 19.

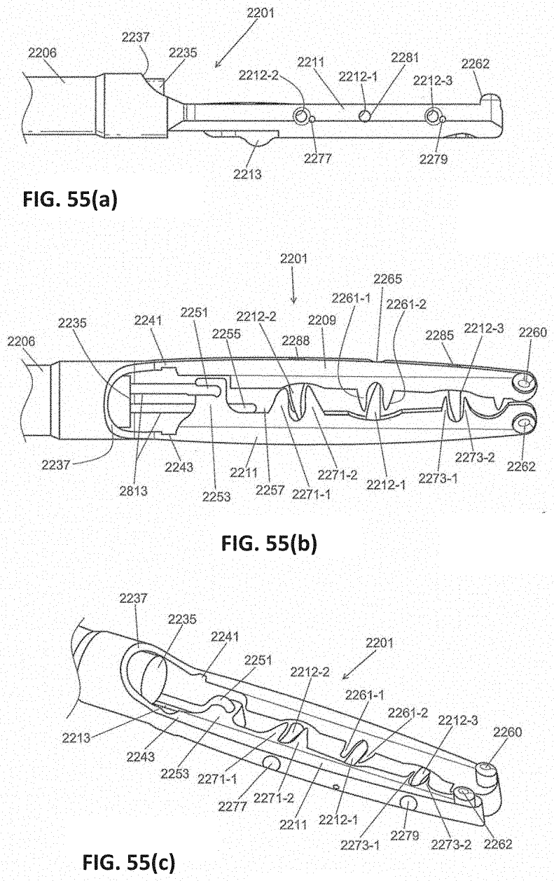

Jaws 2209 and 2211, which can be similar in certain respects to jaws 1981 and 1983 of removal device 19, can be elongated members each made of a medical-grade polymer or a similarly suitable material. Jaws 2209 and 2211 can be pivotally mounted on distal end 2237 of sheath 2206 so that they can be moved towards and away from each other. Sheath 2206 and jaws 2209 and 2211 can form a unitary structure, with jaw 2209 being coupled to sheath 2206 by a living hinge 2241 and with jaw 2211 being coupled to sheath 2206 by a living hinge 2243. Articulation of jaws 2209 and 2211 can be effected using wire 2213, which can include a first end 2251 fixedly coupled to a tab 2253 provided on jaw 2211 and a second end 2255 fixedly coupled to a tab 2257 provided on jaw 2209, with an intermediate portion of wire 2213 passing through sheath 2206 beneath cystoscope 2207 and being fixedly coupled to first member 2215 of handle 2203. In this manner, as first member 2215 can be pivoted towards second member 2217 in a counterclockwise direction indicated by arrow 2218 in FIG. 53, wire 2213 can be moved proximally in tensile fashion, thereby causing jaws 2209 and 2211 to be pivoted towards one another. Conversely, as first member 2215 can be pivoted away from second member 2217, wire 2213 can be moved distally, thereby causing jaws 2209 and 2211 to be pivoted away from one another. As seen best in FIG. 55(b), when device 2201 is viewed from the top, the left end of wire 2813, namely, end 2251, is secured to the right jaw, namely, jaw 2211, and the right end of wire 2213, namely, end 2255, is secured to the left jaw, namely, jaw 2209. It is believed that such an arrangement is advantageous in providing increased leverage for closing jaws 2209 and 2211.

Jaw 2209 can be shaped to include a post 2260 extending upwardly at a distal end 2263 of jaw 2209, and jaw 2211 can be similarly shaped to include a post 2262 extending upwardly at a distal end 2265 of jaw 2211. Posts 2260 and 2262 can be helpful in enabling an operator to visualize the distal end of device 2201, which can facilitate the capture of pressure-attenuating device 17 or the like.

Jaw 2209 can be further shaped to include a couplet of teeth 2261-1 and 2261-2. Teeth 2261-1 and 2261-2 can be disposed at an intermediate location between tab 2257 and post 2260 and can extend generally in the direction of jaw 2211. A transverse opening 2265, which can serve as a relief hole in the manner to become apparent below, can be provided in jaw 2209 between teeth 2261-1 and 2261-2.

Jaw 2211 can be further shaped to include a first couplet of teeth 2271-1 and 2271-2 and a second couplet of teeth 2273-1 and 2273-2, all of which can be disposed between tab 2253 and post 2262. More specifically, teeth 2271-1 and 2271-2 can be positioned so that, when jaws 2209 and 2211 are brought together, teeth 2271-1 and 2271-2 can be located at an intermediate position between tab 2257 and teeth 2261-1 and 2261-2, and teeth 2273-1 and 2273-2 can be positioned so that, when jaws 2209 and 2211 are brought together, teeth 2273-1 and 2273-2 can be located at an intermediate position between teeth 2261-1 and 2261-2 and post 2262. A transverse opening 2277 can be provided in jaw 2211 between teeth 2271-1 and 2271-2, and a transverse opening 2279 can be provided in jaw 2211 between teeth 2273-1 and 2273-2. Openings 2277 and 2279 can function as relief holes in the manner to become apparent below.

Cannulated needle 2212-1 can be fixedly mounted in a transverse opening 2281 provided in jaw 2211 and can be appropriately positioned and dimensioned to be insertable between teeth 2261-1 and 2261-2 when jaws 2209 and 2211 are brought together. In a corresponding fashion, cannulated needle 2212-2 can be fixedly mounted in a transverse opening 2283 provided in jaw 2209 and can be appropriately positioned and dimensioned to be insertable between teeth 2271-1 and 2271-2 when jaws 2209 and 2211 are brought together, and cannulated needle 2212-3 can be fixedly mounted in a transverse opening 2285 provided in jaw 2209 and can be appropriately positioned and dimensioned to be insertable between teeth 2273-1 and 2273-2 when jaws 2209 and 2211 are brought together. It is believed that the present arrangement of teeth and cannulated needles is advantageous in that the teeth can be particularly well-suited to keeping taut the pressure-attenuating device 17 or other object that is to be punctured by the cannulated needles.

Cannulated needles 2212-1 through 2212-3 can be beveled at their respective free ends, and the bevels can extend to a depth that approaches or even exceeds the depths of the teeth on opposing sides of the cannulated needle. A bevel that exceeds the depth of the teeth can be preferred as it can facilitate air loss from the pressure-attenuating device 17 or other object that has been captured and punctured by removal device 2201.

Removal device 2201 can be used in a similar fashion to removal device 19.

It should be understood that the numbers of cannulated needles and teeth disclosed in the present embodiment are merely illustrative and that such numbers can be increased, decreased or otherwise modified. It should also be understood that the size, shape and positioning of such needles and teeth can also be modified. It should further be understood that, although both jaw 2209 and jaw 2211 are described herein as being movable, one could make one of jaws 2209 and 2211 stationary and the other of jaws 2209 and 2211 movable.

As alluded to above, handle 2203, hub 2205, sheath 2206, and jaws 2209 and 2211 can easily be made at low cost using polymeric materials. In addition, needles 2212-1 through 2212-3, and wire 2213 can easily be made at low cost using metallic materials. Moreover, the assembly of removal device 2201 can be achieved economically. Consequently, after a single use of removal device 2201, cystoscope 2207 can be removed and the remainder of removal device 2201 can be disposed. Cystoscope 2207 can then be sterilized for reuse as part of a new removal device 2201. A benefit to making the majority of removal device 2201 single-use is that there is no depreciation in the performance of the device over time. Alternatively, instead of making cystoscope 2207 removable from hub 2205 and sheath 2206 to enable its sterilization and re-use, one could replace one or more of the components of cystoscope 2207 with disposable, single-use components. For example, one could replace the rod lens of cystoscope 2207 with an optical fiber or similar material that is permanently mounted within hub 2205 and sheath 2206.

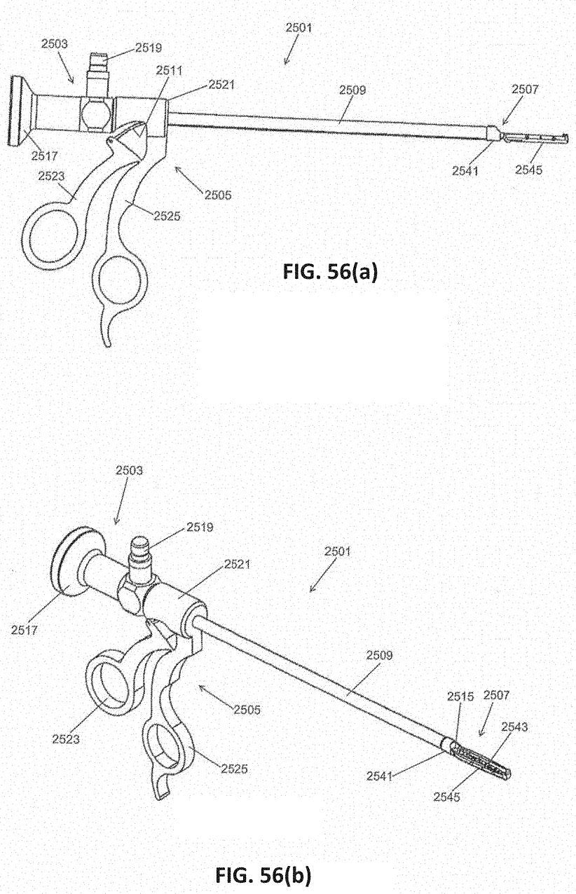

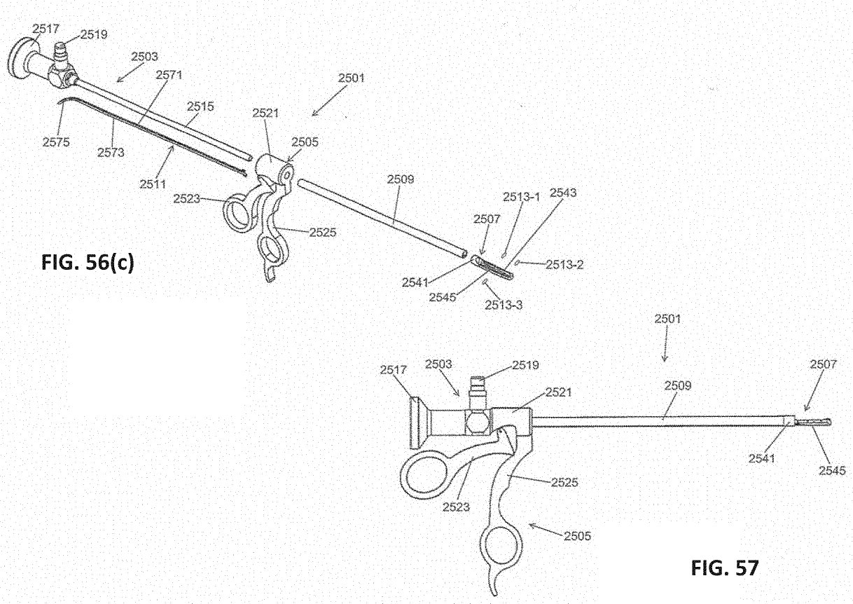

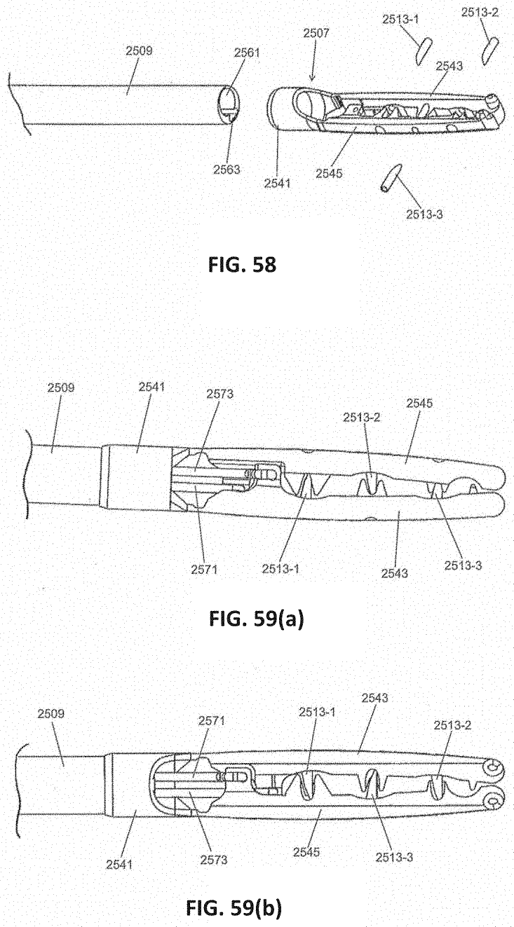

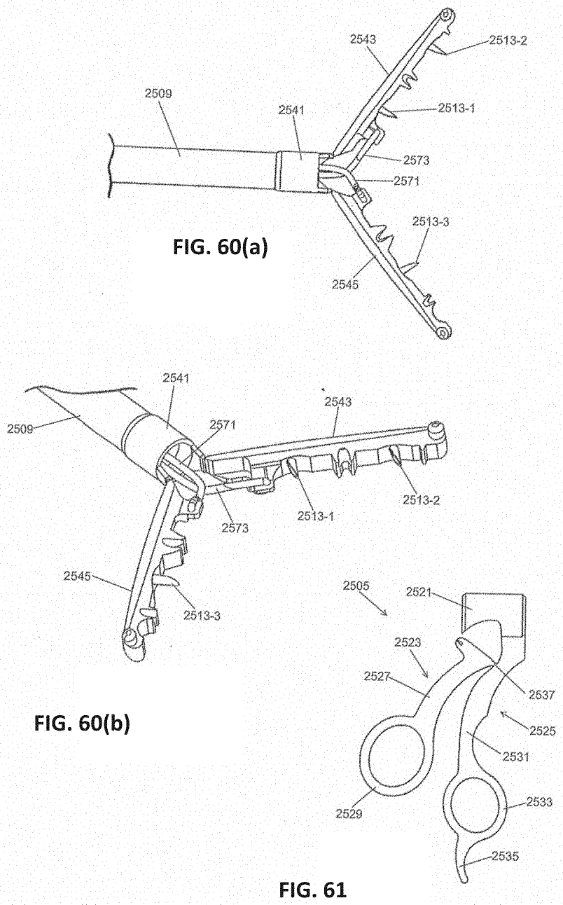

Referring now to FIGS. 56(a) through 56(c), 134, 135, 136(a), 136(b), 137(a), and 137(b), there are shown various views of a fourth alternate embodiment of a removal device, the fourth alternate embodiment of the removal device being represented generally by reference numeral 2501.

Removal device 2501 can be similar in many respects to removal device 2201. A difference between the two removal devices can be that removal device 2501 can be, in its entirety, a disposable single-use device. Removal device 2501 can comprise a cystoscope 2503, a handle assembly 2505, a jaw assembly 2507, a sheath 2509, a wire 2511, and a plurality of cannulated needles 2513-1 through 2513-3.

Cystoscope 2503, which can be made of suitable materials for a single-use, can comprise an optical fiber 2515, an eyepiece 2517, and a light guide 2519.

Handle assembly 2505, which is also shown separately in FIG. 61, can be a unitary structure shaped to comprise a hub 2521, a first member 2523, and a second member 2525. Hub 2521 can be generally tubular in shape and can be dimensioned for insertion therethrough of the distal end of cystoscope 2503. First member 2523 can be shaped to comprise an elongated arm portion 2527. A transversely-extending ring portion 2529, which can be appropriately dimensioned to receive, for example, the thumb of a user, can be disposed at one end of arm portion 2527. Second member 2525 can be shaped to comprise an elongated arm portion 2531. A transversely-extending ring portion 2533, which can be appropriately dimensioned to receive, for example, the forefinger of a user, and a finger rest 2535, which can be appropriately dimensioned to receive, for example, the middle finger of a user, can be disposed at one end of arm portion 2531. The opposite end of arm portion 2531 can be joined to hub 2521. First member 2523 can be coupled to second member 2525 for pivotal movement relative thereto by living hinge member 2537. In this manner, first member 2523 and second member 2525 can be operated much like a pair of scissors, albeit with first member 2523 being regarded as a movable member and with second member 2525 being regarded as a stationary member. It is to be understood, however, that handle assembly 2505 could be modified so that both first member 2523 and second member 2525 are movable.

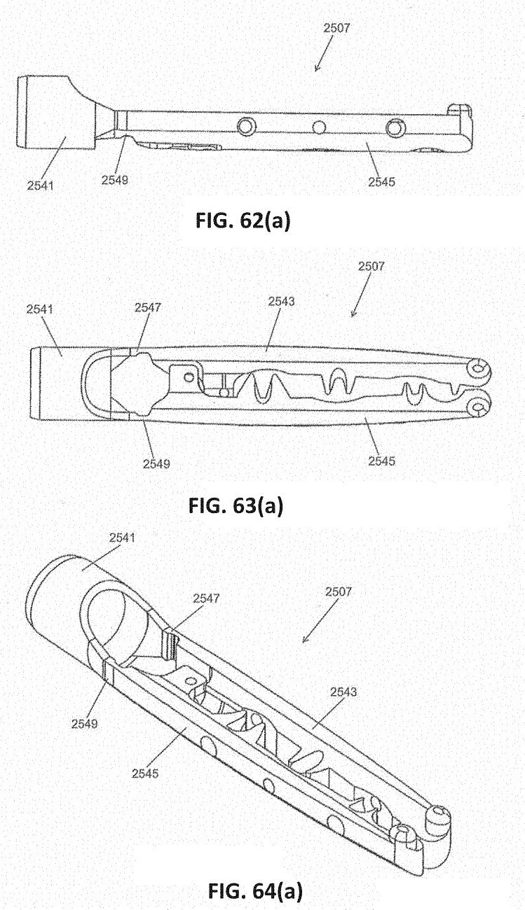

Jaw assembly 2507, which is also shown separately in FIGS. 62(a) through 64(a), can be a unitary structure shaped to comprise a hub 2541, a first jaw 2543, and a second jaw 2545. Hub 2541 can be generally tubular in shape and can be dimensioned to securely receive the distal end of sheath 2509. Jaw 2543, which can be identical to jaw 2209, can be joined to hub 2541 by a living hinge 2547. Jaw 2545, which can be identical to jaw 2211, can be joined to hub 2541 by a living hinge 2549.

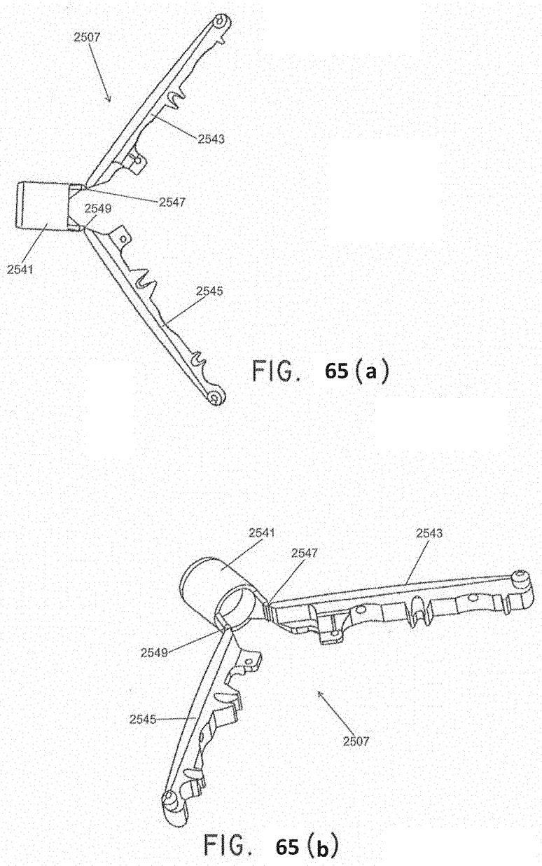

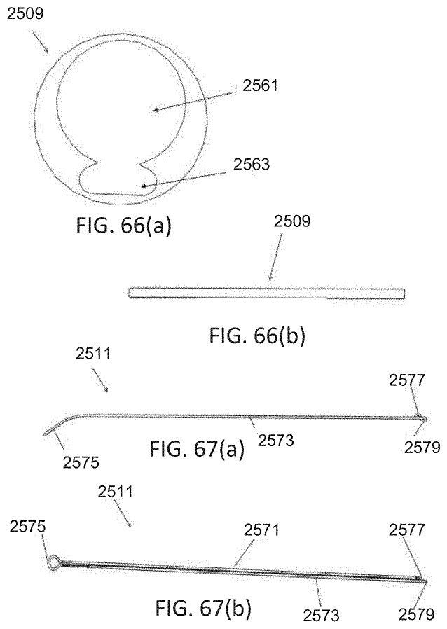

Sheath 2509, which is also shown separately in FIGS. 66(a) and 66(b), can be identical to sheath 2206 and can be shaped to include a first longitudinal cavity 2561 and a second longitudinal cavity 2563. First longitudinal cavity 2561 can be appropriately dimensioned to receive, for example, optical fiber 2515 of cystoscope 2503. Second longitudinal cavity 2563 can be appropriately dimensioned to receive, for example, wire 2511. The outer surface of sheath 2509 is circumferential, allowing for sealing around the sheath when the removal tool is placed in an access device such as 13 and 1291 described herein. The circumferential surface of sheath 2509 is advantageous compared to the outer surface of 1881 and 1901 in removal device 19 described herein. The gap between 1881 and 1901 permits leakage around the valves such as 125 and 91 described herein.

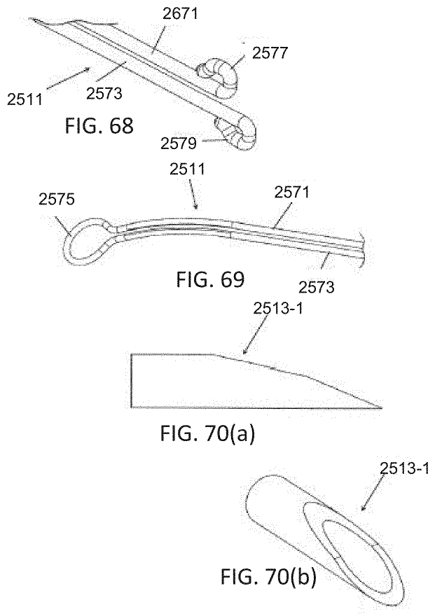

Wire 2511, which is also shown separately in FIGS. 67(a), 67(b), 68, and 69, can be a unitary structure shaped to include a first leg 2571 and a second leg 2573. The proximal ends of first leg 2571 and second leg 2573 can jointly form a loop 2575, which can be secured to first member 2523 of handle assembly 2505. The distal end of first leg 2571 can form a hook 2577, which can be secured to second jaw 2545, and the distal end of second leg 2573 can form a hook 2579, which can be secured to first jaw 2543.

Cannulated needle 2513-1, which can be identical cannulated to needles 2513-2 and 2513-3, can be a unitary structure having the shape shown in FIGS. 70(a) and 70(b).

Additional alternate embodiments to removal device 19 can comprise, in addition to or instead of the cannulated needle, a scissor or similar structure built into jaws 1981 and 1983 to puncture device 17 as teeth 1997 and 2017 hold device 17 or a razor blade or scalpel on one jaw and a receiving slot in the other jaw.

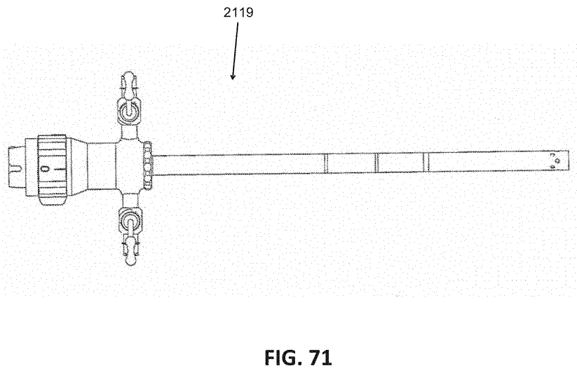

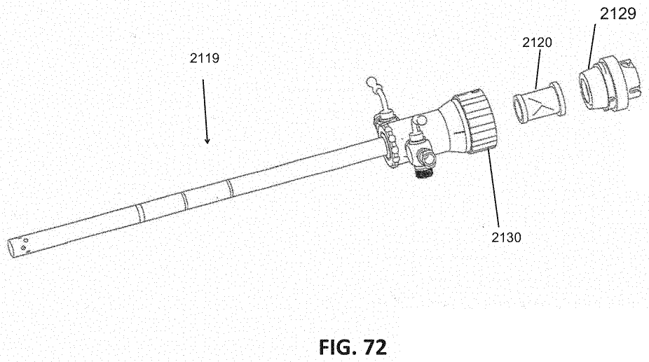

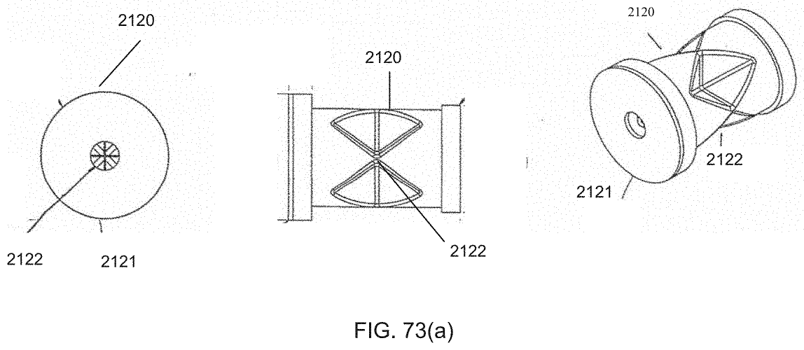

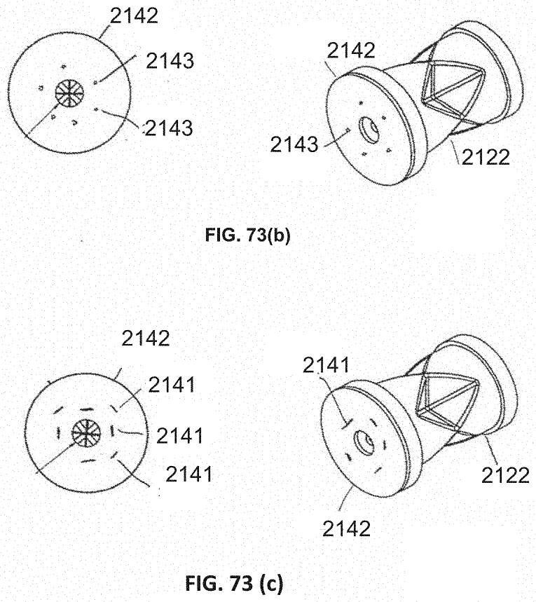

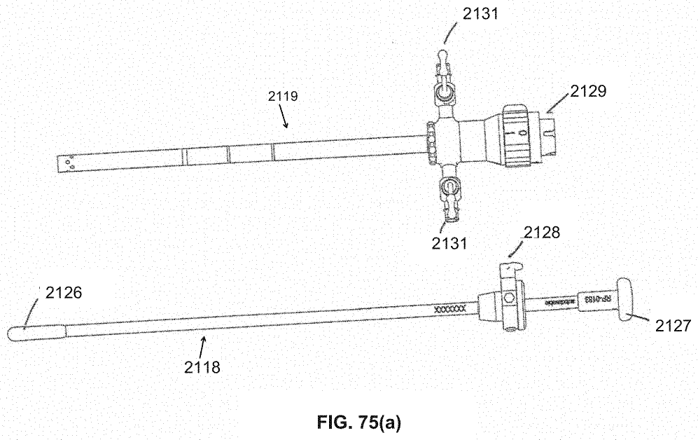

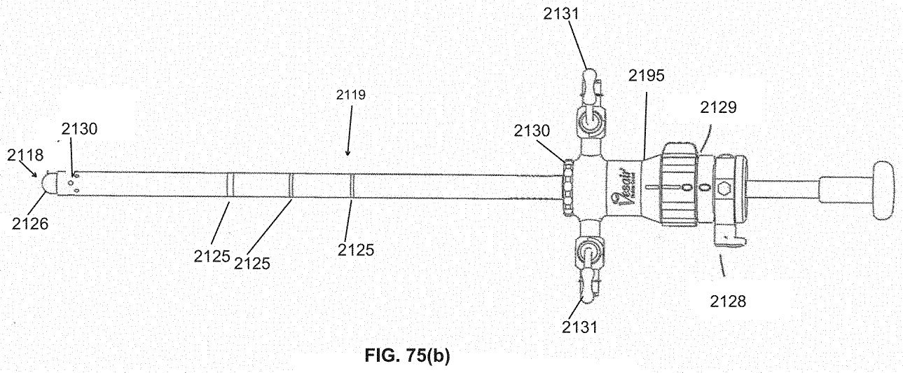

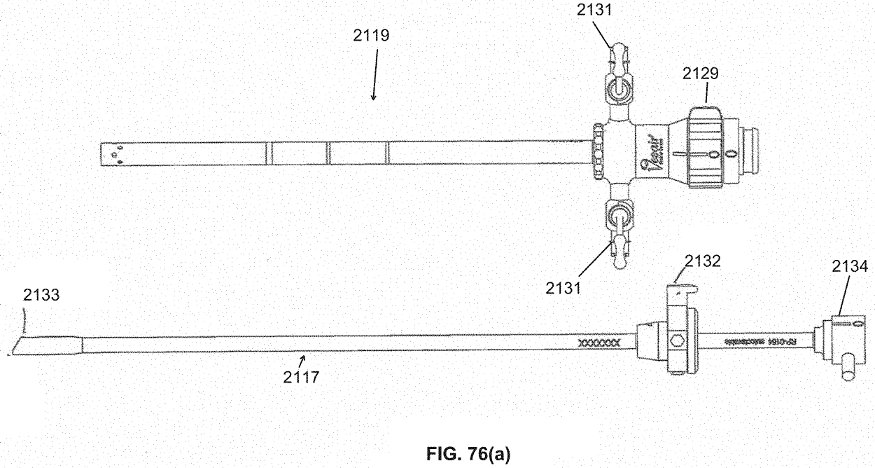

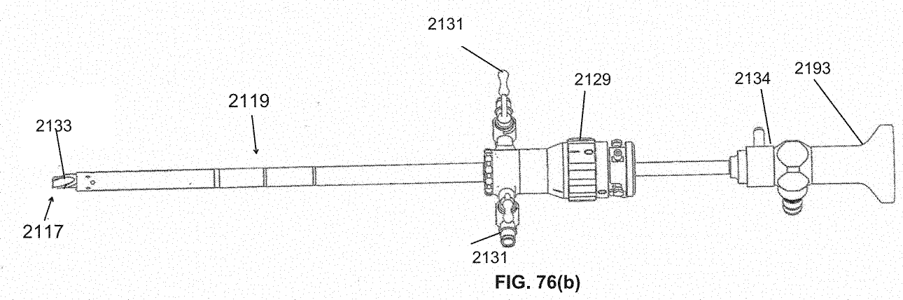

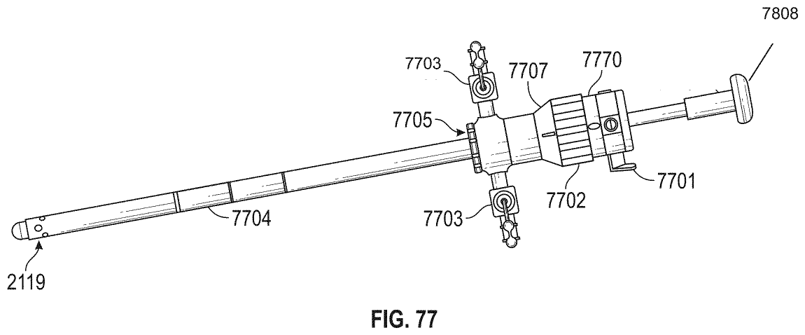

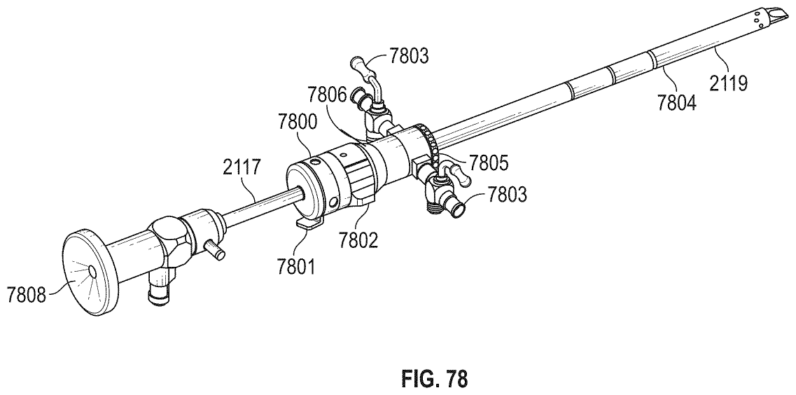

FIG. 71 illustrates a further embodiment of a urethral sheath 2119 that can be used in connection with the various removal devices disclosed herein. In some embodiments, the sheath has a working length of about 166 mm. In some embodiments, the sheath has a working length outer diameter of about 7.5 mm (e.g., 22.5 French). In some embodiments, the sheath has a working length inner diameter of about 6.5 mm (e.g., 19.5 French). As shown in FIGS. 72 and 73(a), the urethral sheath 2119 can include a locking ring 2130, a connecting adapter 2129 and a seal 2120 having at least one connecting adapter 2129 and a valve 2122. As is discussed in additional detail herein, the connecting adapter 2129 is configured to accept and connects to the urethral sheath 2119 various operating tools, including, but not limited to a blunt nosed obturator (such as is shown in FIGS. 75(a), 75(b), and 77) and a visual obturator (such as is shown in FIGS. 76(a), 76(b), and 78).

FIG. 73(a) illustrates various views of the seal 2120. The seal (also called a seal assembly) 2120 includes at least one face (also called a flat seal) 2121 which can be at the distal end of the seal 2120, and is configured to seal the proximal end of the urethral sheath 2119 when a device is within the sheath. The seal 2120 also includes a valve (also called a one way valve) 2122, which can be substantially at the middle of the seal 2120, and is configured to seal the proximal end of the urethral sheath 2119 when there is no device held within the urethral sheath 2119.

FIGS. 73(b) and 73(c) illustrates additional embodiments of the seal (also called a seal assembly) 2142. In some embodiments, a pressure relief valve can be added to the sheath. For example, at least one pressure relief valve can be incorporated into the seal 2142 at the distal end of the seal. Such seal 2142 having a pressure relief valve can advantageously permit fluid, e.g., liquid and/or gas, to evacuate the urethral sheath 2119 while the inflatable implant is being removed through the sheath, e.g., by the jaws of the forceps. In some embodiments, the pressure relief valve would only open during the extraction of the balloon through the urethral sheath 2119 and would normally be closed at typical pressures used or experienced during cystoscopy. In some embodiments, the pressure relief valve opens only when pressures within the urethral sheath 2119 distal to the seal 2142 exceed about 60 cmH₂O, about 55 cmH₂O, about 50 cmH₂O, about 45 cmH₂O, about 40 cmH₂O, about 35 cmH₂O, about 30 cmH₂O, about 25 cmH₂O, about 20 cmH₂O, about 15 cmH₂O, about or 10 cmH₂O. In some embodiments, the pressure relief valve opens only when pressures within the urethral sheath 2119 distal to the seal 2142 exceed about 1 psi. In some embodiments, the pressure relief valve remains closed when pressures within the urethral sheath 2119 distal to the seal 2142 are less than about 60 cmH₂O, about 55 cmH₂O, about 50 cmH₂O, about 45 cmH₂O, about 40 cmH₂O, about 35 cmH₂O, about 30 cmH₂O, about 25 cmH₂O, about 20 cmH₂O, about 15 cmH₂O, about or 10 cmH₂O. In some embodiments, the pressure relief valve remains closed when pressures within the urethral sheath 2119 distal to the seal 2142 are less than about 1 psi. FIG. 73(b) illustrates a pressure relief valve comprising a plurality of holes 2143 in the seal 2142. FIG. 73(c) illustrates a pressure relief valve comprising a plurality of slits 2141 in the seal 2142. Whether slits 2141 or holes 2143, the pressure relief valve can advantageously be configured to remain closed at lower pressures and to pen during the higher pressures experienced by the seal 2142 during balloon removal. In some embodiments, the pressure relief valve comprises a duck bill valve extending from the wall of the sheath near the fluid fill and drain ports of the sheath, e.g., near the stopcocks 2131.

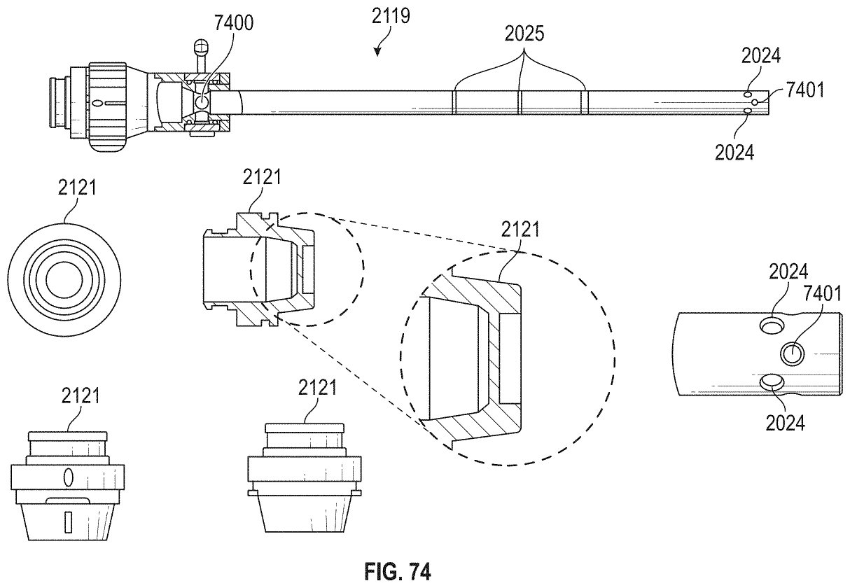

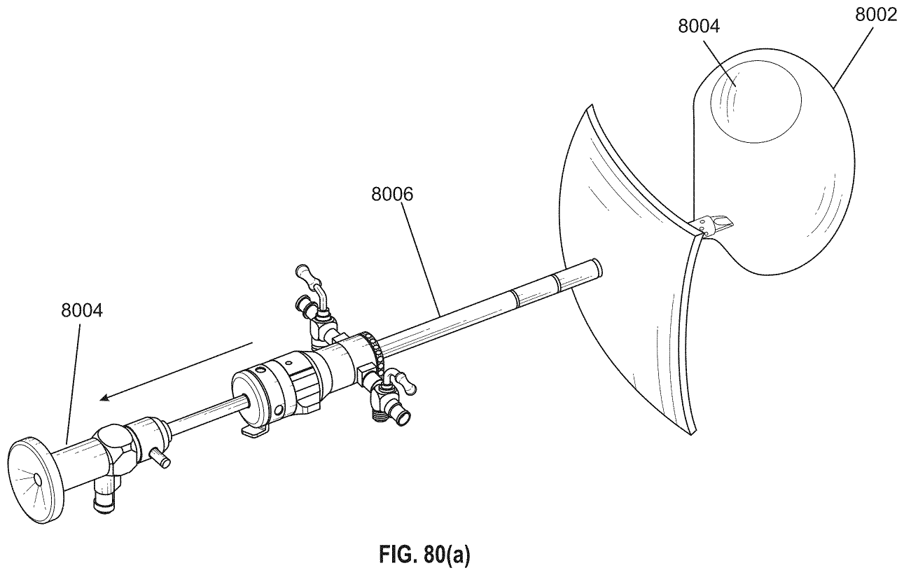

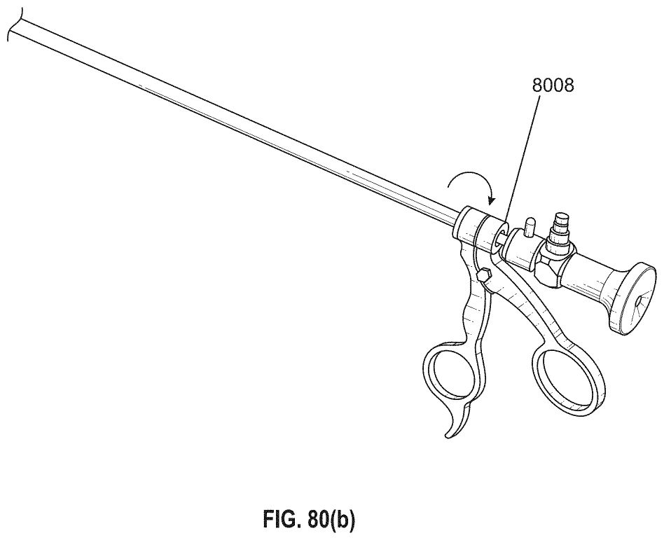

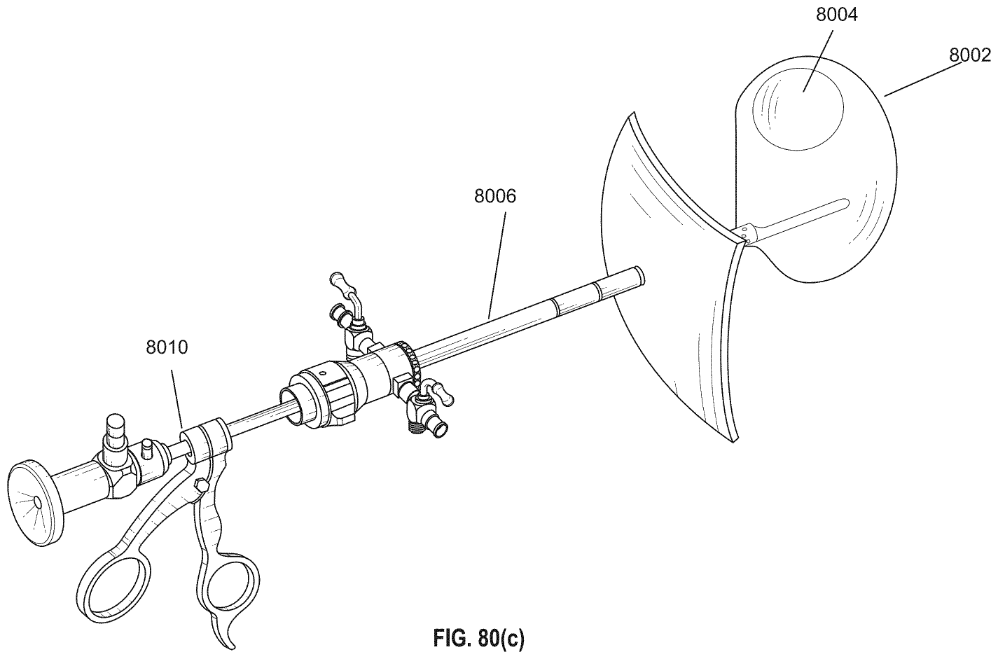

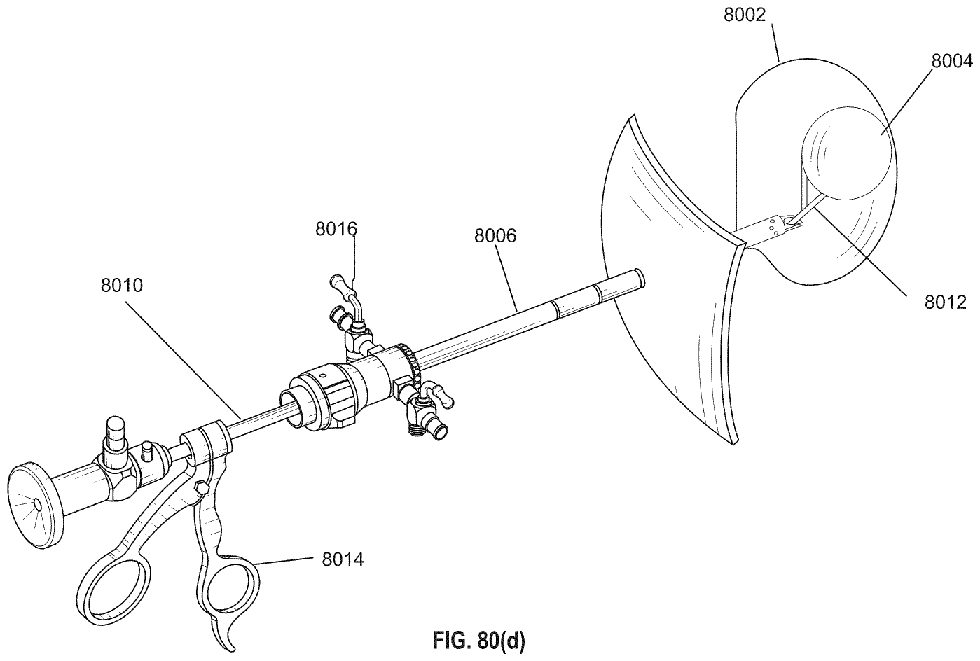

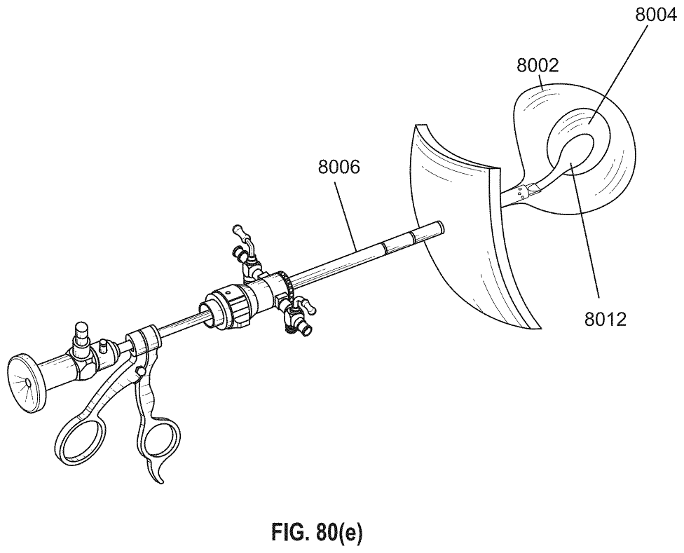

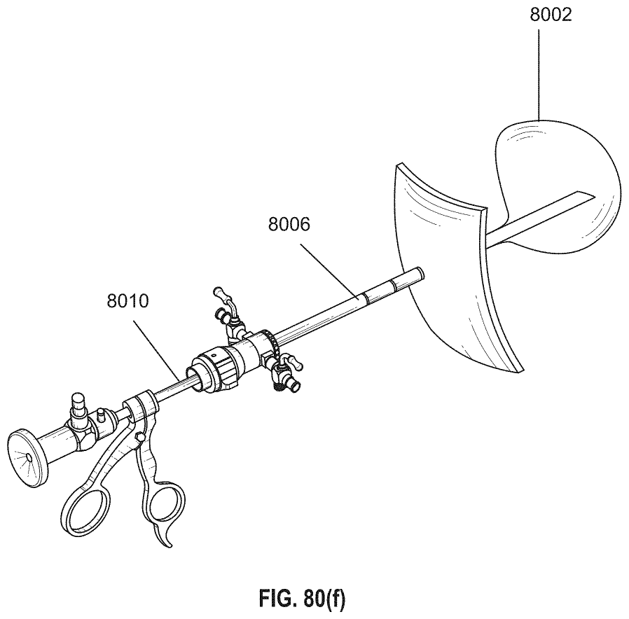

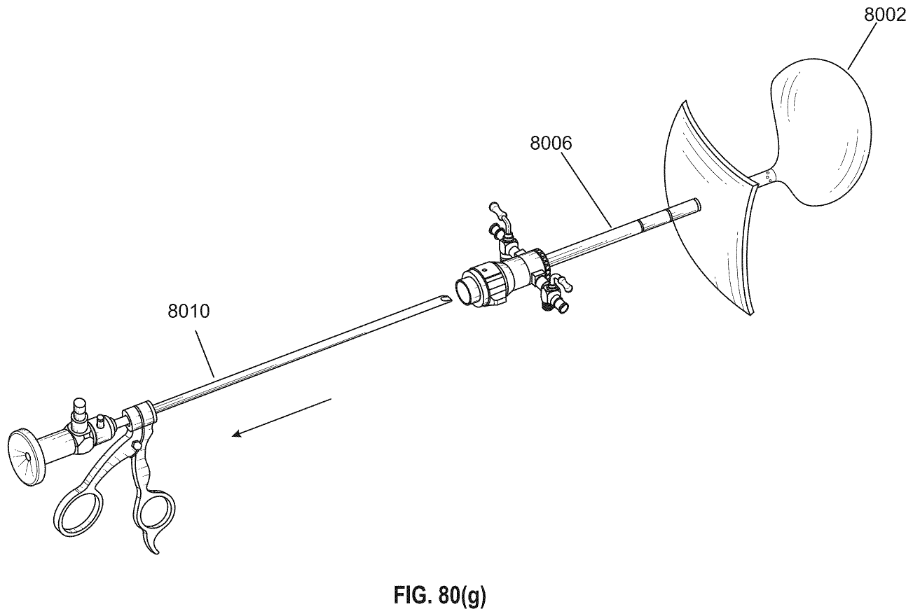

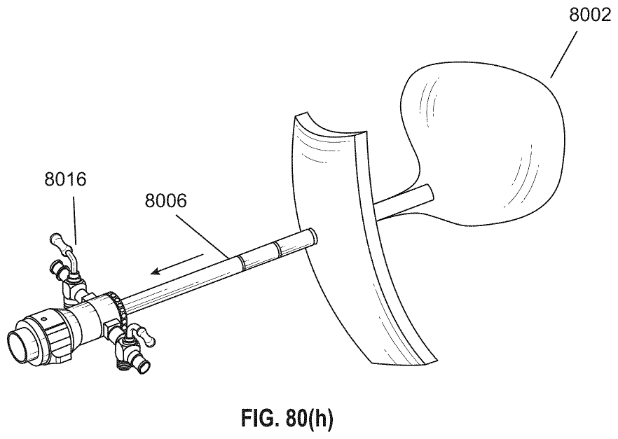

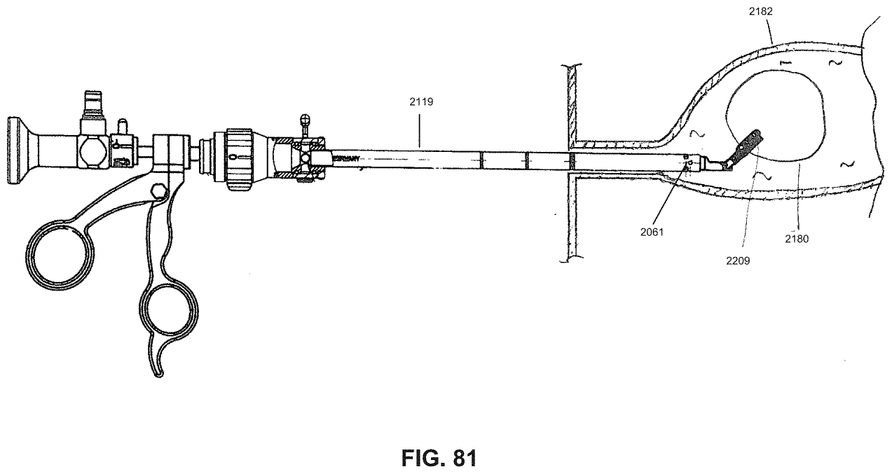

As shown in FIG. 74, the distal end of the urethral sheath 2119 can include a plurality of holes 2024, cross holes for stopcocks 7400, and a distal hole 7401 that can be aligned with cross hole 7400. Such holes 2024 allow for the flow of fluids, e.g., gas, air, PFC, or other substance, out of the balloon during removal (as shown in FIGS. 80 and 81). In some embodiments, the urethral sheath 2119 has no holes at its distal end. However, the holes 2024 can improve, e.g., significantly improve, the ability of fluids to successfully evacuate the balloon when the balloon is being pulled into the distal end of the urethral sheath 2119 during removal. The fluid contained during the balloon can evacuate through the jaws of the forceps, as discussed herein, and out of the sheath holes and into the bladder. When no holes are present, the fluids being vented from the balloon must exit only the distal end of the urethral sheath 2119, which can become temporarily or permanently plugged, obscured, or blocked by the presence of the balloon. In some embodiments, the urethral sheath 2119 includes a series of reference markings 2125 on its longitudinal surface to allow the user a gauge of how deep the device has been inserted into the patient, e.g., into the patient's bladder. In some embodiments, the urethral sheath 2119 has three reference markings 2125. In some embodiments, the urethral sheath 2119 has more than three reference markings 2125, e.g., four, five six, seven, eight, nine, ten, eleven, or 12 reference markings 2125. In some embodiments, the urethral sheath 2119 has fewer than three reference markings 2125, e.g., two or one reference markings 2125. FIG. 74 also shows embodiments of different view of the seal 2121.

As referenced herein, FIG. 75(a) illustrates a urethral sheath 2119 and a blunt nose obturator 2118, side by side. The urethral sheath 2119 includes, among other things two stopcocks 2131 and a connecting adapter 2129. The blunt nose obturator 2118 includes a blunt nose obturator tip 2126 at its distal end and a blunt nose obturator release button 2128 and a blunt nose obturator knob 2127 at its proximal end. FIG. 75(b) and FIG. 77 illustrates two embodiments of the blunt nose obturator 2118 inserted into the urethral sheath 2119, ready for use. The blunt nose obturator tip 2126 of the blunt nose obturator 2118 extends slightly past the distal end of the urethral sheath 2119, allowing for atraumatic, or less traumatic, insertion into and through a physical lumen, such as a urethra. The blunt nose obturator release button 2128 of the blunt nose obturator 2118 is configured to attach to the connecting adapter 2129 of the urethral sheath 2119 and releasably hold the blunt nose obturator 2118 to the urethral sheath 2119. The holes 2130 can be located on the distal tip. In some embodiments, as shown in FIG. 77, the blunt nose obturator 2118 inserted in a sheath 2119 can have a connecting adapter 7700, a blunt nose obturator 2118, a obturator release button 7701, a locking ring 7702, stopcocks 7703, reference markings 7704, a sheath 2119, a retaining nut 7705, a single-use seal 7706, and a connecting adaptor 7707.

FIG. 76(a) illustrates a reusable metal urethral sheath 2119 and a visual obturator 2117, side by side. As discussed herein, the urethral sheath 2119 includes a connecting adapter 2129 that can be used to releasably hold the visual obturator 2117 to the urethral sheath 2119. The visual obturator 2117 includes a visual obturator tip 2133 at its distal end and a visual obturator release button 2132 and a scope adapter 2134 at its proximal end. FIG. 76(b) and FIG. 78 illustrate two different embodiments of the devices, both with the visual obturator 2117 in place, or inserted, within the urethral sheath 2119. The devices can have a sheath 2119, reference markings 2125, a retaining nut 2191, 7805, stopcocks 2131, 7803 locking ring 2130, 7805 obturator release button 2128, 7801 cytoscope 2193, 7808, visual obturator 2117, connecting adapter 2129, 7802 and a single-use seal 2195. The visual obturator tip 2133 extends a distance past the distal end of the urethral sheath 2119. Additionally, the visual obturator release button 2128 connects to the locking ring 2130 to hold the visual obturator 2117 in place within the urethral sheath 2119. As can be seen, the visual obturator tip 2133 extends a distance past the distal end of the locking ring 2130 to hold the visual obturator 2117 in place within the urethral sheath 2119. As shown in FIG. 77, a scope 7808 can be inserted into and through the scope adapter 2134 to allow visualization through the visual obturator 2117. In some embodiments, as shown in FIG. 78, the visual obturator 2117 inserted in a sheath 2119 can have a connecting adapter 7800, a visual obturator 2117, a obturator release button 7801, a locking ring 7802, stopcocks 7803, reference markings 7804, a sheath 2119, a retaining nut 7805, a single-use seal 7806, and a cystoscope 7808.

The following describes insertion of the devices shown in FIGS. 77 and 78. The sheath's outer diameter can be 22.5 Fr. The sheath's outer diameter can be 19.5 Fr. The sheath's working length can be 166 cm. The urethral sheath 2119 can be provided non-sterile and can be used when accessing the bladder. When provided non-sterile, the urethral sheath 2119 can advantageously be cleaned and steam sterilized prior to use to prevent harm, e.g., infection, to the patient. Next, the urethral sheath 2119 and any ancillary components can be placed on the sterile field. Then, inflow tubing can be connected to one stopcock 2131 and outflow tubing connected to the other stopcock 2131. Inflow tubing can be used to deliver fluid to one or more of the device system, e.g., including, among other things, the urethral sheath 2119, and the patient. Outflow tubing can be used to remove fluid from one or more of the device system, e.g., including, among other things, the urethral sheath 2119, and the patient.

To prepare the patient's body for system insertion, the opening to a physical lumen, e.g., the urethra, can be exposed. In female patients, the user parts the labia to expose the meatus. Holding the body of the urethral sheath 2119, the tip 2126 of the blunt nose obturator 2118 can then be aligned with the opening of the urethra. After successful alignment the blunt nose obturator tip 2126 of the blunt nose obturator 2118 can be inserted into the patient, e.g., into the urethra, by slowly advancing the sheath in a straight and steady motion.

As discussed herein, the urethral sheath 2119 can have two stopcocks 2131, one for inflow and the other for outflow. The two stopcocks 2131 can be interchangeable. To control inflow and outflow, the levers located on the stopcock 2131 can be used. In some embodiments, fluid control can be performed with the blunt nose obturator 2118 in place within the urethral sheath 2119. In some embodiments, fluid control can be performed with the visual obturator 2117 in place within the urethral sheath 2119. Fluid control can be performed with the visual obturator in place. Lubricious placement of the urethral sheath 2119 can advantageously be provided by lubricating the tip of the sheath or turn inflow on, e.g., using the inflow stopcock 2131, as the urethral sheath 2119 is introduced into the patient, e.g., the patient's urethra. To minimize the chance of bladder perforation during sheath insertion, the user should advance the sheath with care.

Once the urethral sheath 2119 is placed, the blunt nose obturator 2118 can be removed by holding the urethral sheath 2119 steady, e.g., with one hand, and depressing the blunt nose obturator release button 2128, e.g., with the other hand. The blunt nose obturator 2118 can then be withdrawn, e.g., gently withdrawn, from the urethral sheath 2119, keeping the urethral sheath 2119 steady and in place.

To place the visual obturator 2117, the urethral sheath 2119 remaining in the physical lumen, e.g., the urethra, is held steady, e.g., with one hand, while the visual obturator tip 2133 is slowing inserted into the urethral sheath 2119, e.g., using the other hand. The visual obturator 2117 can be secured in place by securing the visual obturator release button 2132 to the connecting adapter 2129, e.g., by clicking it into place. When using the visual obturator 2117, the urethral sheath 2119 can be inserted under direct visualization.

As discussed herein, the body of the urethral sheath 2119 can have three reference markings 2125. These reference markings 2125 can be used to help the user externally note the location of the urethral sheath 2119 within the urethra following its positioning under cystoscopic guidance.

In some embodiments, it can be advantageous to ensure that the bladder appears distended prior to removing the visual obturator 2117 and inserting a balloon delivery catheter or a removal device 2001, as disclosed herein. If the bladder is not sufficiently full or distended, the inflow stopcock 2131 can be used to inject additional fluid into the bladder until the desired volume has been reached.

The following below describe use of a removal device as disclosed herein to remove an inflatable implant from within a patient's bladder. As disclosed herein, the balloon removal system is comprised of optical forceps and a cystoscope, e.g., a 0 degree wide-angle cystoscope. The balloon removal system can be provided non-sterile. When provided non-sterile, the balloon removal system can advantageously be cleaned and steam sterilized prior to use to prevent harm, e.g., infection, to the patient.

A cystoscope, e.g., within the visual obturator 2117, can be used to visually confirm that the bladder is adequately filled and the inflatable implant can be seen before inserting the balloon removal system through the urethral sheath 2119. Following cystoscopy, the visual obturator 2117 can be removed while keeping the urethral sheath 2119 in place. Then, as disclosed elsewhere herein, the wide angel cystoscope can be slid into the channel of the removal device and locked in position, e.g., by turning the locking mechanism to the right (or in any other way locking the two together). A light source and camera can then be attached to the cystoscope

The balloon removal system can then be aligned with the opening of the sheath. Once aligned, the optical forceps can be advanced, e.g., jaws first, into the urethral sheath 2119, using direct visualization. The optical forceps can be visually advanced to the location of the inflatable implant. To locate and grasp the inflatable implant, fluid can need to be drained from the bladder by using one of the stopcocks 2131 on the urethral sheath 2119 (note: in some embodiments, the balloon removal system must be pulled back into the sheath before fluid can be successfully drained from the bladder). Once the inflatable implant can be seen directly or in front of the jaws, the jaws of the optical forceps can be opened and the jaws advanced to or by the inflatable implant.

Once the jaws have been successfully placed by or next to the inflatable implant, the jaws of the optical forceps can be closed by closing the handle of the optical forceps. Closing the handle can cause the jaws to close on the inflatable implant, puncturing the inflatable implant and permitting it to deflate. Once the inflatable implant has been punctured, visual confirmation of escaping air bubbles can be possible. Following successfully disruption of the inflatable implant, e.g., puncturing, the inflatable implant can be allowed to deflate through the holes in the jaws for an amount of time, e.g., about 15-20 seconds.

After deflation of the balloon, while holding the sheath and keeping the optical forceps handle closed, the optical forceps and the inflatable implant can be slowly pulled through the sheath. If a high force is encountered while pulling the optical forceps (and inflatable implant) through the sheath, the optical forceps can be advanced back into the bladder under direct visual guidance. Then, the user can visually confirm the balloon has been punctured before reattempting removal. If the inflatable implant becomes dislodged from the optical forceps while pulling the optical forceps through the sheath, the optical forceps can be reinserted through the sheath and into the bladder. Then the user can locate and re-grasp the inflatable implant and continue its removal.

After removal of the inflatable implant, the inflatable implant can advantageously be inspected. If there is any concern that the entire inflatable implant was not removed, the optical forceps can be reinserted, and any remaining pieces of inflatable implant located, grasped, and removed. If no other procedure is necessary, the urethral sheath 2119 can be removed. If placing a new balloon, the fluid in the bladder can be replaced by turning the inflow on using one of the stopcocks 2131 prior to inserting the balloon.

The following below describes use of a deflecting removal device as disclosed herein, e.g., such as is shown in FIG. 23, to remove an inflatable implant from within a patient's bladder. As disclosed herein, the balloon removal system is comprised of deflectable optical forceps and a cystoscope, e.g., a 30 degree long cystoscope. The balloon removal system can be provided non-sterile. When provided non-sterile, the balloon removal system can advantageously be cleaned and steam sterilized prior to use to prevent harm, e.g., infection, to the patient.

A scope 2000, e.g., within the visual obturator 2117, can be used to visually confirm that the bladder is adequately filled and the inflatable implant can be seen before inserting the balloon removal system through the urethral sheath 2119. Following cystoscopy, the visual obturator 2117 can be removed while keeping the urethral sheath 2119 in place. Then, as disclosed elsewhere herein, the 30 degree long cystoscope can be slid into the channel of the removal device and locked in position, e.g., by turning the locking mechanism to the right (or in any other way locking the two together). A light source and camera can then be attached to the cystoscope

The balloon removal system can then be aligned with the opening of the sheath. Once aligned, the optical forceps can be advanced, e.g., jaws first, into the urethral sheath 2119, using direct visualization. The jaws can be maintained in a closed and undeflected configuration during insertion into the urethral sheath 2119, e.g., by squeezing the handle of the optical forceps closed. The optical forceps can be visually advanced to the location of the inflatable implant. To locate and grasp the inflatable implant, fluid can need to be drained from the bladder by using one of the stopcocks 2131 on the urethral sheath 2119 (note: in some embodiments, the balloon removal system must be pulled back into the sheath before fluid can be successfully drained from the bladder). Once the inflatable implant can be seen directly or in front of the jaws, the jaws of the optical forceps can be opened, e.g., by releasing the handles, and the jaws advanced to or by the inflatable implant. To improve maneuverability within the patient's bladder and/or access to the inflatable implant, the deflectable jaws can be caused to deflect vertically.

Once the jaws have been successfully placed by or next to the inflatable implant, e.g., such that the deflecting jaws have made contact with the inflatable balloon, the jaws of the optical forceps can be closed by closing the handle of the optical forceps. Closing the handle can cause the jaws to close on the inflatable implant, puncturing the inflatable implant and permitting it to deflate (shown in FIG. 80(f)). Once the inflatable implant has been punctured, visual confirmation of escaping air bubbles can be possible. Following successfully disruption of the inflatable implant, e.g., puncturing, the inflatable implant can be allowed to deflate through the holes in the jaws for an amount of time, e.g., about 15-20 seconds. Before withdrawing the deflectable jaws into the urethral sheath 2119, the deflection of the jaws can advantageously be eliminated such that the jaws are axially aligned with the urethral sheath 2119, e.g., by further squeezing the handle.

After deflation of the balloon, while holding the sheath and keeping the optical forceps handle closed (and the deflectable jaws aligned with the urethral sheath 2119), the optical forceps and the inflatable implant can be slowly pulled through the sheath (shown in FIG. 81). If a high force is encountered while pulling the optical forceps (and inflatable implant) through the sheath, the optical forceps can be advanced back into the bladder under direct visual guidance. Then, the user can visually confirm the balloon has been punctured before reattempting removal. If the inflatable implant becomes dislodged from the optical forceps while pulling the optical forceps through the sheath, the optical forceps can be reinserted through the sheath and into the bladder. Then the user can locate and re-grasp the inflatable implant and continue its removal.

After removal of the inflatable implant, the inflatable implant can advantageously be inspected. If there is any concern that the entire inflatable implant was not removed, the optical forceps can be reinserted, and any remaining pieces of inflatable implant located, grasped, and removed. If no other procedure is necessary, the urethral sheath 2119 can be removed. If placing a new balloon, the fluid in the bladder can be replaced by turning the inflow on using one of the stopcocks 2131 prior to inserting the balloon.

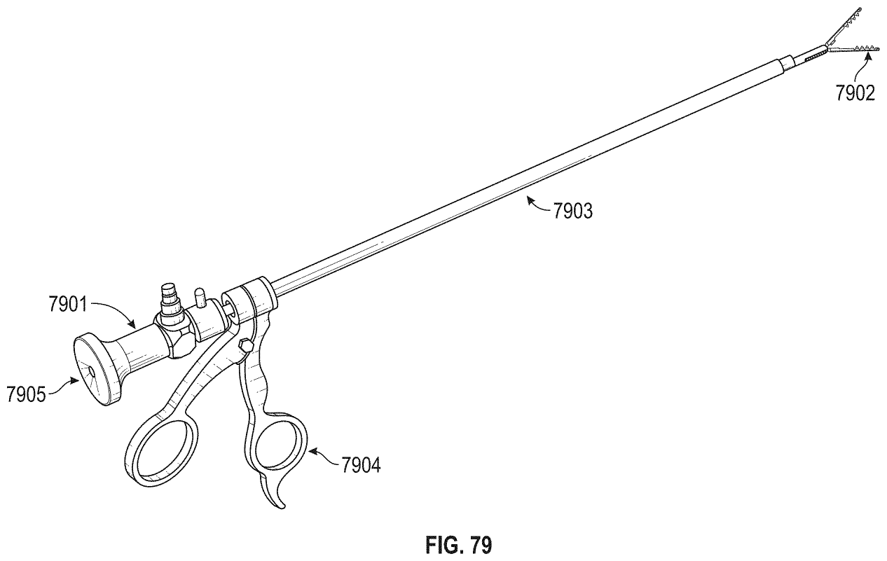

FIG. 79 shows a removal device with a lens eyepiece 7905, handle 7904, optical forceps 7903, cystoscope 7901,and jaws 7902 that is used in the following removal instructions. In some embodiments, the cytoscope can be a 0 degrees wide angle cytoscope or a 30 degrees long cytoscope. In some embodiments, the optical forceps can be deflectable.

FIGS. 80(a)-80(g) show how the removal device can be used to remove the inflatable device or balloon 8004 from the bladder 8002. The removal device can be provided non-sterile and can be cleaned and steam sterilized prior to use. As shown in FIG. 80(a), the user visually confirms the bladder 8002 is adequately filled and the inflatable implant or balloon 8004 can be seen before inserting the removal through the sheath 8006. Following cystoscopy, the visual obturator 8004is removed, keeping the sheath 8006 in place. The cystoscope 8008 can be slid into the channel of the optical forceps and locked in position by turning the locking mechanism to the right as shown in FIG. 80(b). Then, the user can attach the light source and camera to the Cystoscope 8006. As shown in FIG. 80(c), the removal device 8010 can then be aligned with the opening of the sheath 8006. The optical forceps can then be advanced into the sheath 8006 using direct visualization to the location of the balloon 8004. As shown in FIG. 80(d), to locate and grasp the balloon 8004, fluid may need to be drained from the bladder 8002 by utilizing one of the stopcocks 8016 on the sheath 8006. Next, the jaws of optical forceps 8012 are opened when the balloon 8004 can be seen directly on or in front of the jaws 8012. To grasp balloon 8004, the open jaws 8012 can be gently pushed against Balloon 8004 making contact before squeezing the handles 8014 closed. To drain fluid from the removal device 8010, the device 8010 can be pulled back into the sheath 8006. As shown in FIG. 80(e), the optical forceps handle 8014 can be closed in order to make the jaws of the forceps 8012 grasp and deflate the balloon 8004. Once the balloon 8004 has been punctured, visual confirmation of escaping air bubbles is possible. Air bubbles can escape through holes located in the sheath as well. As shown in FIG. 80(f), wait 15-20 seconds while balloon 8004 deflates through the holes in the jaws 8012 and then the user can visually confirm the balloon 8004 is no longer inflated. As shown in FIG. 80(g), while holding the sheath 8006 and keeping the optical forceps handle 8014 closed, the user can slowly pull the optical forceps and the balloon 8004 through the sheath 8006. The balloon 8004 can tear if the forceps handle is prematurely released during this step. Note that if a high force is encountered while pulling the optical forceps through the sheath 8006, one can advance the optical forceps 8012 into the bladder 8002 under direct visual guidance and visually confirm the balloon 8004 has been punctured before reattempting removal. If the balloon 8004 becomes dislodged from the optical forceps 8012 while pulling the forceps 8012 through the sheath 8006, reinsert the forceps through the sheath 8006 and into the bladder 8002 to locate and re-grasp the balloon 8004 and remove. Once the balloon 8004 is removed, the user can inspect the removed balloon 8004. If there is any concern that the entire balloon 8004 has not been removed, reinsert the optical forceps 8012, locate, grasp and remove any balloon 8004 pieces. As shown in FIG. 80(h), the sheath 8006 then can be removed. If placing a new balloon 8004, one can replace the fluid in the bladder 8002 by turning the inflow on using one of the stopcocks 8016.

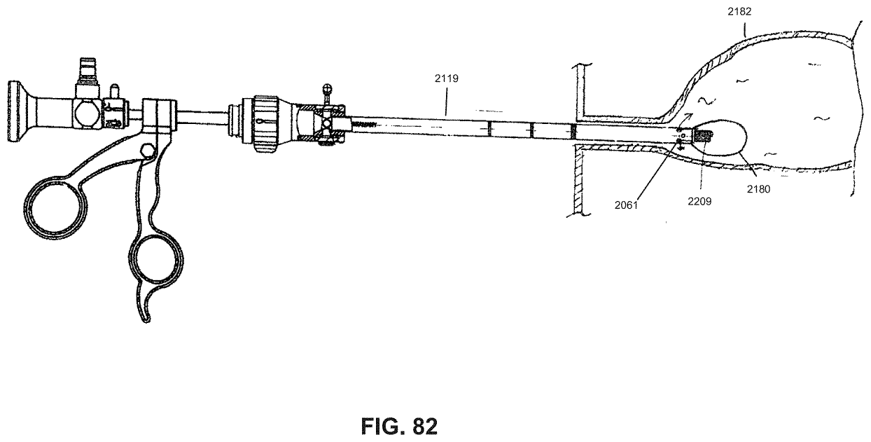

FIG. 81 shows the jaws 2209 contacting or grabbing the balloon or inflatable implant 2180 within a bladder 2182. FIG. 82 shows the retrieval of the inflatable implant 2180 from the bladder 2182 by withdrawing the shaft 2184 (not shown) from the sleeve or sheath 2119. Additionally, once the inflatable implant 2180 is deflating and pulled within the sheath, air can travel out of the holes or vent holes 2061 and/or into the sheath 2119. The holes 2061 can allow air to escape when the valves are closed or when the sheath is occluded, thereby preventing a creation of a pressure differential which prevents a user from removing the implant 2180 out of the sheath 2119

The foregoing description and examples has been set forth merely to illustrate the disclosure and are not intended as being limiting. Each of the disclosed aspects and embodiments of the present disclosure can be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure can occur to persons skilled in the art and such modifications are within the scope of the present disclosure. Furthermore, all references cited herein are incorporated by reference in their entirety.

Terms of orientation used herein, such as “top,” “bottom,” “horizontal,” “vertical,” “longitudinal,” “lateral,” and “end” are used in the context of the illustrated embodiment. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular” or “cylindrical” or “semi-circular” or “semi-cylindrical” or any related or similar terms, are not required to conform strictly to the mathematical definitions of circles or cylinders or other structures, but can encompass structures that are reasonably close approximations.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Conjunctive language, such as the phrase “at least one of X, Y, and Z,” unless specifically stated otherwise, is otherwise understood with the context as used in general to convey that an item, term, etc. can be either X, Y, or Z. Thus, such conjunctive language is not generally intended to imply that certain embodiments require the presence of at least one of X, at least one of Y, and at least one of Z.

The terms “approximately,” “about,” and “substantially” as used herein represent an amount close to the stated amount that still performs a desired function or achieves a desired result. For example, in some embodiments, as the context can dictate, the terms “approximately”, “about”, and “substantially” can refer to an amount that is within less than or equal to 10% of the stated amount. The term “generally” as used herein represents a value, amount, or characteristic that predominantly includes or tends toward a particular value, amount, or characteristic. As an example, in certain embodiments, as the context can dictate, the term “generally parallel” can refer to something that departs from exactly parallel by less than or equal to 20 degrees.

Unless otherwise explicitly stated, articles such as “a” or “an” should generally be interpreted to include one or more described items. Accordingly, phrases such as “a device configured to” are intended to include one or more recited devices. Such one or more recited devices can be collectively configured to carry out the stated recitations. For example, “a processor configured to carry out recitations A, B, and C” can include a first processor configured to carry out recitation A working in conjunction with a second processor configured to carry out recitations B and C.

The terms “comprising,” “including,” “having,” and the like are synonymous and are used inclusively, in an open-ended fashion, and do not exclude additional elements, features, acts, operations, and so forth. Likewise, the terms “some,” “certain,” and the like are synonymous and are used in an open-ended fashion. Also, the term “or” is used in its inclusive sense (and not in its exclusive sense) so that when used, for example, to connect a list of elements, the term “or” means one, some, or all of the elements in the list.

Overall, the language of the claims is to be interpreted broadly based on the language employed in the claims. The language of the claims is not to be limited to the non-exclusive embodiments and examples that are illustrated and described in this disclosure, or that are discussed during the prosecution of the application.

Although systems and methods for treating a patient using a compressible, pressure-attenuating device have been disclosed in the context of certain embodiments and examples, this disclosure extends beyond the specifically disclosed embodiments to other alternative embodiments and/or uses of the embodiments and certain modifications and equivalents thereof. Various features and aspects of the disclosed embodiments can be combined with or substituted for one another in order to form varying modes of systems and methods for treating a patient using a compressible, pressure-attenuating device. The scope of this disclosure should not be limited by the particular disclosed embodiments described herein.

Certain features that are described in this disclosure in the context of separate implementations can be implemented in combination in a single implementation. Conversely, various features that are described in the context of a single implementation can be implemented in multiple implementations separately or in any suitable subcombination. Although features can be described herein as acting in certain combinations, one or more features from a claimed combination can, in some cases, be excised from the combination, and the combination can be claimed as any subcombination or variation of any subcombination.

While the methods and devices described herein can be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various embodiments described and the appended claims. Further, the disclosure herein of any particular feature, aspect, method, property, characteristic, quality, attribute, element, or the like in connection with an embodiment can be used in all other embodiments set forth herein. Any methods disclosed herein need not be performed in the order recited. Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some embodiments, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each embodiment. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some embodiments can be performed using the sequence of operations described herein, while other embodiments can be performed following a different sequence of operations.

Moreover, while operations can be depicted in the drawings or described in the specification in a particular order, such operations need not be performed in the particular order shown or in sequential order, and all operations need not be performed, to achieve the desirable results. Other operations that are not depicted or described can be incorporated in the example methods and processes. For example, one or more additional operations can be performed before, after, simultaneously, or between any of the described operations. Further, the operations can be rearranged or reordered in other implementations. Also, the separation of various system components in the implementations described herein should not be understood as requiring such separation in all implementations, and it should be understood that the described components and systems can generally be integrated together in a single product or packaged into multiple products. Additionally, other implementations are within the scope of this disclosure.

Some embodiments have been described in connection with the accompanying figures. Certain figures are drawn and/or shown to scale, but such scale should not be limiting, since dimensions and proportions other than what are shown are contemplated and are within the scope of the embodiments disclosed herein. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, any methods described herein can be practiced using any device suitable for performing the recited steps.

The methods disclosed herein can include certain actions taken by a practitioner; however, the methods can also include any third-party instruction of those actions, either expressly or by implication. For example, actions such as “positioning an electrode” include “instructing positioning of an electrode.”

In summary, various embodiments and examples of systems and methods for treating a patient using a compressible, pressure-attenuating device have been disclosed. Although the systems and methods for treating a patient using a compressible, pressure-attenuating device have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Thus, the scope of this disclosure should not be limited by the particular disclosed embodiments described herein, but should be determined only by a fair reading of the claims that follow.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure.

1. A removal device comprising:
at least one manually-actuatable member; and
at least two opposing deflectable jaws, at least one of the at least two opposing deflectable jaws being movable by actuation of the at least one manually-actuatable member, wherein at least one of the at least two opposing deflectable jaws comprises a gripping member and wherein at least one of the at least two opposing deflectable jaws comprises a puncturing member, wherein the at least two opposing deflectable jaws are configured to deflect vertically.

2. The removal device of claim 1, wherein the puncturing member comprises a cannulated needle.

3. The removal device of claim 2, wherein the at least two opposing deflectable jaws are configured to open laterally.

4. The removal device of claim 1, further comprising at least one pull wire coupled to the at least one manually-actuatable member and the at least two opposing deflectable jaws.

5. The removal device of claim 4, wherein the at least one pull wire comprises a first pull wire and a second pull wire.

6. The removal device of claim 5, wherein the first pull wire is configured to open the at least two opposing deflectable jaws and the second pull wire is configured to deflect the at least two opposing deflectable jaws.

7. The removal device of claim 1, further comprising a single pull wire coupled to the at least one manually-actuatable member and the at least two opposing deflectable jaws.

8. The removal device of claim 7, the single pull wire being configured to both open the at least two opposing deflectable jaws and deflect the at least two opposing deflectable jaws.

9. The removal device of claim 8, wherein the single pull wire includes axial section, a curved section and a neck section, the curved section causing the at least two deflectable jaws to deflect vertically and the neck section controlling when the least two opposing deflectable jaws are open.

10. The removal device of claim 1, wherein the jaw opposing the jaw with the puncturing member includes an opening configured to receive the cannulated needle when the at least two opposing deflectable jaws are in a closed position.

11. The removal device of claim 1, wherein each of the at least two opposing deflectable jaws comprises a puncturing member and an opening, the opening of each of the at least two jaws opposing deflectable jaws being adapted to receive the puncturing member on the opposing deflectable jaw when the at least two opposing deflectable jaws are in a closed position.

12. The removal device of claim 11, wherein each puncturing member comprises a cannulated needle.

13. The removal device of claim 1, wherein the gripping member comprises a row of teeth.

14. The removal device of claim 1, wherein both of the at least two opposing deflectable jaws include gripping members.

15. The removal device of claim 14, wherein the gripping members comprise a row of teeth.

16. The removal device of claim 15, wherein in the closed position there is a gap between the rows of teeth of the at least two opposing deflectable jaws.

17. The removal device of claim 1, wherein the puncturing member is generally perpendicular to the jaw.

18. The removal device of claim 1, further comprising a camera.

19. A removal device comprising:
(a) at least one manually-actuatable member; and
(b) at least two opposing deflectable jaws, at least one of the at least two opposing deflectable jaws being movable by actuation of the at least one manually-actuatable member, wherein at least one of the at least two opposing deflectable jaws comprises a gripping member and wherein at least one of the at least two opposing deflectable jaws comprises a puncturing member, wherein the at least two opposing deflectable jaws are configured to deflect vertically.

20. The removal device of claim 19, wherein the jaw opposing the jaw with the puncturing member includes an opening configured to receive the puncturing member when the at least two opposing deflectable jaws are in a closed position.

21. The removal device of claim 20, wherein the at least two opposing deflectable jaws are configured to open laterally.

22. The removal device of claim 19, further comprising at least one pull wire coupled to the at least one manually-actuatable member and the at least two opposing deflectable jaws.

23. The removal device of claim 22, wherein the at least one pull wire comprises a first pull wire and a second pull wire.

24. The removal device of claim 23, wherein the first pull wire is configured to open the at least two opposing deflectable jaws and the second pull wire is configured to deflect the at least two opposing deflectable jaws.

25. The removal device of claim 19, further comprising a single pull wire coupled to the at least one manually-actuatable member and the at least two opposing deflectable jaws.

26. The removal device of claim 25, the single pull wire being configured to both open the at least two opposing deflectable jaws and deflect the at least two opposing deflectable jaws.

27. The removal device of claim 26, wherein the single pull wire includes axial section, a curved section and a neck section, the curved section causing the at least two deflectable jaws to deflect vertically and the neck section controlling when the least two opposing deflectable jaws are open.

28. The removal device of claim 19, wherein each of the at least two opposing deflectable jaws comprises a puncturing member and an opening, the opening of each of the at least opposing two deflectable jaws being adapted to receive the puncturing member on the opposing jaw when the at least two opposing deflectable jaws are in a closed position.

29. The removal device of claim 19, wherein the gripping member comprises a row of teeth.

30. The removal device of claim 19, wherein both of the at least two opposing deflectable jaws include gripping members.

31. The removal device of claim 30, wherein the puncturing member is a cannulated needle.

32.-67. (canceled)
